(19) United States
(12) Patent Application Publication
(10) Pub. No.: US 2011/0028471 A1
(43) Pub. Date: Feb. 3, 2011

(54) COMBINATION THERAPY 238

(75) Inventors: Juliane Jürgensmeier, Macclesfield (GB); Georgina Speake, Macclesfield (GB)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 12/918,650

(22) PCT Filed: Feb. 20, 2009
(86) PCT No.: PCT/GB09/50167

§ 371 Date: Oct. 22, 2010

Related U.S. Application Data
(60) Provisional application No. 61/030,299, filed on Feb. 21, 2008.
(60) Provisional application No. 61/048,605, filed on Apr. 29, 2008.

(51) Int. Cl. A61K 31/5377; A61P 35/00

(52) U.S. Cl. 514/232.5; 514/234.2

(57) ABSTRACT

There is provided a combination product comprising a VEGFR tyrosine kinase inhibitor and a m TOR-selective kinase inhibitor, and methods for the production of an anti-cancer effect in a patient, which is accordingly useful in the treatment of cancer in a patient.

The present invention relates to a combination product, as defined herein, comprising a VEGFR tyrosine kinase inhibitor and a mTOR-selective kinase inhibitor, and to methods for the production of an anti-cancer effect in a patient, which is accordingly useful in the treatment of cancer in a patient. More specifically the present invention relates to; a combination product, as defined herein, comprising AZD2171 or ZD6474 and a mTOR-selective kinase inhibitor; a combination product, as defined herein, comprising a kit of parts comprising AZD2171 or ZD6474 and a mTOR-selective kinase inhibitor; use of the combination product, as defined herein, in the treatment of cancer; a method of treating cancer comprising administering the combination product, as defined herein, to a patient. The combination product, as defined herein, and methods of the invention are also useful in the treatment of other diseases mediated by VEGF and/or mTOR.

Normal angiogenesis plays an important role in a variety of processes including embryonic development, wound healing and several components of female reproductive function. Undesirable or pathological angiogenesis has been associated with disease states including diabetic retinopathy, psoriasis, cancer, rheumatoid arthritis, atheroma, Kaposi's sarcoma and haemangioma (Fan et al, 1995, Trends Pharmacol. Sci. 16: 57-66; Folkman, 1995, Nature Medicine 1: 27-31). Alteration of vascular permeability is thought to play a role in both normal and pathological physiological processes (Cullinan-Bove et al, 1993, Endocrinology 133: 829-837; Senger et al, 1993, Cancer and Metastasis Reviews, 12: 303-324). Several polypeptides with in vitro endothelial cell growth promoting activity have been identified including, acidic and basic fibroblast growth factors (aFGF & bFGF) and vascular endothelial growth factor (VEGF). By virtue of the restricted expression of its receptors, the growth factor activity of VEGF, in contrast to that of the FGFs, is relatively specific towards endothelial cells. Recent evidence indicates that VEGF is an important stimulator of both normal and pathological angiogenesis (Jakeman et al, 1993, Endocrinology, 133: 848-859; Kolch et al, 1995, Breast Cancer Research and Treatment, 36:139-155) and vascular permeability (Connolly et al, 1989, J. Biol. Chem. 264: 20017-20024). Antagonism of VEGF action by sequestration of VEGF with antibody can result in inhibition of tumour growth (Kim et al, 1993, Nature 362: 841-844).

Receptor tyrosine kinases (RTKs) are important in the transmission of biochemical signals across the plasma membrane of cells. These transmembrane molecules characteristically consist of an extracellular ligand-binding domain connected through a segment in the plasma membrane to an intracellular tyrosine kinase domain. Binding of ligand to the receptor results in stimulation of the receptor-associated tyrosine kinase activity which leads to phosphorylation of tyrosine residues on both the receptor and other intracellular molecules. These changes in tyrosine phosphorylation initiate a signalling cascade leading to a variety of cellular responses. To date, at least nineteen distinct RTK subfamilies, defined by amino acid sequence homology, have been identified. One of these subfamilies is presently comprised by the fms-like tyrosine kinase receptor, Flt-1 (also referred to as VEGFR-1), the kinase insert domain-containing receptor, KDR (also referred to as VEGFR-2 or Flk-1), and another fms-like tyrosine kinase receptor, Flt-4 (also referred to as VEGFR-3). Two of these related RTKs, Flt-1 and KDR, have been shown to bind VEGF with high affinity (De Vries et al, 1992, Science 255: 989-991; Terman et al, 1992, Biochem. Biophys. Res. Comm. 1992, 187: 1579-1586). Binding of VEGF to these receptors expressed in heterologous cells has been associated with changes in the tyrosine phosphorylation status of cellular proteins and calcium fluxes.

VEGF is a key stimulus for vasculogenesis and angiogenesis. This cytokine induces a vascular sprouting phenotype by inducing endothelial cell proliferation, protease expression and migration, and subsequent organisation of cells to form a capillary tube (Keck, P. J., Hauser, S. D., Krivi, G., Sanzo, K., Warren, T., Feder, J., and Connolly, D. T., Science (Washington D.C.), 246: 1309-1312, 1989; Lamoreaux, W. J., Fitzgerald, M. E., Reiner, A., Hasty, K. A., and Charles, S. T., Microvasc. Res., 55: 29-42, 1998; Pepper, M. S., Montesano, R., Mandroita, S. J., Orci, L. and Vassalli, J. D., Enzyme Protein, 49: 138-162, 1996). In addition, VEGF induces significant vascular permeability (Dvorak, H. F., Detmar, M., Claffey, K. P., Nagy, J. A., van de Water, L., and Senger, D. R., (Int. Arch. Allergy Immunol., 107: 233-235, 1995; Bates, D. O., Heald, R. I., Curry, F. E. and Williams, B. J. Physiol. (Lond.), 533: 263-272, 2001), promoting formation of a hyper-permeable, immature vascular network which is characteristic of pathological angiogenesis.

It has been shown that activation of KDR alone is sufficient to promote all of the major phenotypic responses to VEGF, including endothelial cell proliferation, migration, and survival, and the induction of vascular permeability (Meyer, M., Clauss, M., Lepple-Wienhues, A., Waltenberger, J., Augustin, H. G., Ziche, M., Lanz, C., Büttner, M., Rziha, H-J., and Dehio, C., EMBO J., 18: 363-374, 1999; Zeng, H., Sanyal, S, and Mukhopadhyay, D., J. Biol. Chem., 276: 32714-32719, 2001; Gille, H., Kowalski, J., Li, B., LeCouter, J., Moffat, B, Zioncheck, T. F., Pelletier, N. and Ferrara, N., J. Biol. Chem., 276: 3222-3230, 2001).

A VEGFR tyrosine kinase inhibitor is any agent that inhibits VEGF receptor tyrosine kinase including small molecule receptor tyrosine kinase inhibitors and antibodies. Examples include vandetanib (ZD6474), cediranib (AZD2171), NEXAVAR™ (sorafenib, Bayer), SUTENT™ (sunitinib, Pfizer), PTK787 (vatalanib), AMG-706 (motesanib), CEP-7055, E7080, AG-013736 (axitinib), GW-786034 (pazopanib), SU14813, BAY 57-9352, KRN-951, ABT-869, OSI-930, CP-547,632, BMS 582664, BIBF-1120, CHIR-258, AEE-788, CHIR-265 and ZK-304709.

Quinazoline derivatives which are inhibitors of VEGF receptor tyrosine kinase are described in International Patent Applications Publication Nos. WO 98/13354 and WO 01/32651. In WO 98/13354 and WO 01/32651 compounds are described which possess activity against VEGF receptor tyrosine kinase (VEGF RTK) whilst possessing some activity against epidermal growth factor (EGF) receptor tyrosine kinase (EGF RTK). ZD6474 is 4-(4-bromo-2-fluoroanilino)-6-methoxy-7-(1-methylpiperidin-4-ylmethoxy)quinazoline:

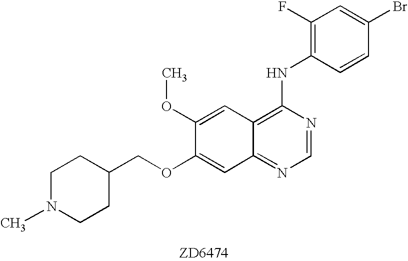

ZD6474 is also known as vandetanib and as ZACTIMA™ (Trade mark of the AstraZeneca Group of Companies).

ZD6474 falls within the broad general disclosure of WO 98/13354 and is exemplified in WO 01/32651. ZD6474 is a potent inhibitor of VEGF RTK and also has some activity against EGF RTK. ZD6474 has been shown to elicit broad-spectrum anti-tumour activity in a range of models following once-daily oral administration (Wedge S R, Ogilvie D J, Dukes M, et al. ZD6474 inhibits vascular endothelial growth factor signaling, angiogenesis, and tumour growth following oral administration. Cancer Res 2002; 62:4645-4655).

Quinazoline derivatives which are inhibitors of VEGF receptor tyrosine kinase are described in International Patent Application Publication No. WO 00/47212. AZD2171 is described in WO 00/47212 and is Example 240 therein. AZD2171 is 4-(4-fluoro-2-methyl-1H-indol-5-yloxy)-6-methoxy-7-(3-(pyrrolidin-1-yl)propoxy)quinazoline:

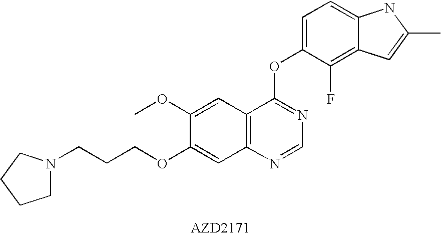

AZD2171 is also known as cediranib and RECENTIN™ (Trade mark of the AstraZeneca Group of Companies).

AZD2171 shows excellent activity in the in vitro (a) enzyme and (b) HUVEC assays that are described in WO 00/47212 (pages 80-83). The AZD2171 IC₅₀ values for inhibition of isolated KDR (VEGFR-2), Flt-1 (VEGFR-1) and Flt-4 (VEGFR-3) tyrosine kinase activities in the enzyme assay were <2 nM, 5±2 nM and ≦3 nM respectively. AZD2171 inhibits VEGF-stimulated endothelial cell proliferation potently (IC₅₀ value of 0.4±0.2 nM in the HUVEC assay), but does not inhibit basal endothelial cell proliferation appreciably at a >1250 fold greater concentration (IC₅₀ value is >500 nM). The growth of a Calu-6 tumour xenograft in the in vivo solid tumour model described in WO 00/47212 (page 83) was inhibited by 49%**, 69%*** and 91%*** following 28 days of once-daily oral treatment with 1.5, 3 and 6 mg/kg/day AZD2171 respectively (P**<0.01, P***<0.0001; one-tailed t test). AZD2171 has been shown to elicit broad-spectrum anti-tumour activity in a range of models following once-daily oral administration, (Wedge et al., 2005, Cancer Research 65: 4389-4440).

We have found that AZD2171, as well as producing an antiangiogenic and/or vascular permeability reducing effect by virtue of inhibiting KDR, can have an additional direct antiproliferative effect on tumour cells mediated by inhibition of stem cell factor receptor tyrosine kinase (SCF RTK, commonly known as c-Kit). We have found that AZD2171 inhibits c-Kit and it is expected that AZD2171 will inhibit mutated and wild-type c-Kit. c-Kit and its ligand SCF have been found in numerous solid and haematological malignancies, including gastrointestinal stromal tumours, primary brain tumours such as glioblastoma, glioma and medulloblastoma, small cell lung cancer (SCLC), malignant mesothelioma, tumours of the testis such as seminoma and testicular teratocarcinoma, tumours of the ovary such as dysgerminoma and gonadoblastoma, chronic myelogenous leukaemia (CML), acute myelogenous leukaemia (AML) and mastocytosis (see for example Jnl. Clin. Oncol., 2004, 22, 4514-4522). c-Kit has also been found in hepatocellular carcinoma, (Am J Clin Pathol. 2005 July; 124(1):31-6), and colorectal carcinoma, (Case Reports Tumour Biol. 1993; 14(5):295-302). c-Kit is an important signal transduction inhibitor in certain cancers such as gastrointestinal tumours (GIST), (Bumming et al, 2003 Br J Cancer 89, 460-464), small cell lung cancer (SCLC), (Pott et. al., 2003, Annals of Oncology 14: 894-879), and chronic myelogenous leukaemia (CML), (Goselink et al. 1992, Blood 80, 750-757 and Muroi et al, 1995, Leuk Lymphoma 16, 297-305). c-Kit is also an important signal transduction inhibitor in soft tissue sarcomas like leiomyosarcoma.

mTOR (mammalian target of rapamycin) is a growth factor and nutrient-sensitive regulator of cell growth affecting a wide range of cellular functions including translation, transcription, mRNA turnover, protein stability, actin cytoskeleton reorganisation and autophagy (Guertin, et al., Cancer Cell, 12, 9-22). It lies downstream of the PI3K/AKT signalling cascade which is dysregulated in a significant proportion of cancers (PI3KCA mutations and amplifications, PTEN mutations and deletions, AKT overexpression) (Marone 2008, BBA, Lopiccolo et al, Drug Resist Update (2008). mTOR is a 289 KDa protein, member of the PI3-kinase like kinase (PIKK) family of proteins containing proteins such as DNA-PKcs (DNA dependent protein kinase) and ATM (Ataxia-telangiectasia mutated). A growth factor such as insulin, after binding to its receptor, activates PI3K via the protein IRS-1. PI3K converts the substrate PIP2 into PIP3, activating downstream proteins PDK-1 and AKT. mTOR is involved in two complexes; mTORC1 containing mTOR, raptor, GβL and PRAS40, is rapamycin-sensitive and activated by AKT and mTORC2 containing mTOR, rictor, PROTOR, GβL and Sin1 is insensitive to rapamycin (Sarbassov et al, Curr Biol. 14:1296-302 (2004)). mTORC1-dependent phosphorylation of S6-kinase (p70S6K) allows translation of ribosomal proteins via activation of its substrate ribosomal protein S6 (rpS6). mTORC1 also phosphorylates the translation initiation factor 4E-BP1 (PHAS-1), preventing its inhibitory binding to eIF4E and allowing the formation of an active eIF4F translation complex (Proud, Biochem J. 403:217-34 (2007)). p70S6 kinase negatively regulates mTOR activation by phosphorylating the protein IRS-1 which promotes its degradation by the proteosome. The mTORC2 complex directly phosphorylates and activates the upstream kinase AKT on serine 473. It also phosphorylates proteins involved in the cytoskeleton such as paxillin (Sarbassov et al, Mol. Cell. 22; 159-68 (2006), Jacinto and Hall, Nature Rev Mol Cell Biol, 4, 117-126, (2005)). Finally, there is evidence that mTOR signalling regulates endothelial cell proliferation stimulated by vascular endothelial cell growth factor (VEGF), and partially controls VEGF synthesis through effects on the expression of hypoxia-inducible factor-1α (HIF-1α) (Hudson et al., MolCell Biol, 2002, 22, 7004-7014, Dancey, Exp Opinlnvest Drugs, 2005, 14, 313-328; Seeliger et al, Cancer Metastasis Rev. 2007 December; 26(3-4):611-21. Finally, recent evidence suggests that mTOR is activated in Kaposi sarcoma, promoting endothelial cell proliferation. The viral protein vGPCR is central to Kaposi sarcomagenesis: In HUVEC (human umbilical vascular endothelial cells), it promotes the relocalisation and activation of AKT, induces tuberin (TSC2) phosphorylation and inactivation, thereby promoting the activation of mTOR and its downstream molecules, including p70 S6K and 4EBP1. The resulting proliferative effect on endothelial cells is independent of VEGF and could not be abolished by a VEGF-blocking antibody (Montaner 2007). Therefore, tumour angiogenesis may depend on mTOR kinase signalling in both a VEGF-dependent and independent manner.

The PI3K/AKT signalling cascade is often dysregulated in cancers (Marone 2008, BBA, Lopiccolo et al, Drug Resist Update (2008). In addition, a number of genetic disease have now been linked to the PI3K/AKT/mTOR pathway: Cowden syndrome, tuberous sclerosis, Peutz-Jeghers syndrome, and Birt-Hogg-Dubé syndrome are due to mutations or deletions of proteins in the PI3K/AKT/mTOR pathway (PTEN, TSC1 and 2, LKB1 and folliculin, respectively) (Lopiccolo, Jozwiak Lancet Oncol. 2008 January; 9(1):73-9). Some of these genetic disease usually develop as hamartomas which are very vascularised benign tumours. However, patients with Cowden syndrome, Peutz-Jeghers syndrome, and Birt-Hogg-Dubé syndrome have a significantly increased risk of cancer (breast and endometrial cancer in Cowden patients, gastrointestinal cancers in PJS patients, renal cancer in BHD patients). Finally, the PI3K/AKT/mTOR pathway is implicated in a number of non malignant pathologies such as polycystic kidney disease (Masoumi 2007), chronic obstructive pulmonary disease (COPD) (Krymskaya BioDrugs. 2007; 21(2):85-95) and ocular conditions such as age related macular degeneration (AMD), glaucoma and uveitis.

Inhibition of mTORC1 results in cell cycle arrest and cell growth inhibition (Reviewed in refs. Burnett et al; Huang and Houghton, Curr Opin Pharmacol, 3, 371-377 (2003); Sawyers, Cancer Cell, 4, 343-348 (2003)). Rapamycin (sirolimus or Rapamune™) and rapalogues (everolimus (RAD001 or Certican™), temsirolimus (CCI-779) and deforolimus (AP23573) bind to the FK506 binding protein, FKBP12. Subsequently, the complex of FKBP12/Rapamycin binds to the FRB domain of mTOR within the mTORC1 complex, inhibiting its downstream signalling. Rapamycin, potently inhibits proliferation or growth of normal cells (smooth muscle cells, T-cells) and tumour cells from rhabdomyosarcoma, neuroblastoma, glioblastoma and medulloblastoma, small cell lung cancer, osteosarcoma, pancreatic carcinoma and breast and prostate carcinoma (Faivre et al. 2006). Consequently, rapamycin has been approved as an immunosuppressant and for use in the prevention of organ rejection (reviewed in Neuhaus, et al., Liver Transplantation, 7, 473-484 (2001); Woods and Marks, Ann Rev Med, 55, 169-178 (2004)). In addition, rapamycin and rapalogues are also in clinical development in oncology (Faivre 2006 Nat Rev Drug Disc). However, results from clinical trials so far have been less positive than expected. A reason for this may be that inhibition of mTORC1 alone can stimulate AKT phosphorylation by inhibiting the negative feedback loop between p70S6K and IRS1 (Faivre 2006, Cloughesy 2008). This finding suggests that inhibitors of the kinase activity of mTOR should preferably inhibit both mTORC1 and mTORC2 complexes.

Rapamycin potentiates the cytotoxicity of a number of cytotoxic agents such as cisplatin, camptothecin and doxorubicin (Huang and Horton). Potentiation of ionising radiation induced cell killing has also been observed following inhibition of mTOR (Eshleman, et al., Cancer Res, 62, 7291-7297 (2002)). Rapamycin analogues are showing evidence of efficacy in treating cancer, either alone or in combination with other therapies (Bjornsti and Houghton; Huang and Houghton; Huang and Houghton).

The present invention provides a combination product comprising a VEGFR tyrosine kinase inhibitor and a mTOR-selective kinase inhibitor. The combination product of the invention is useful in a method for the production of an anti-cancer effect in a patient, which is accordingly useful in the treatment of cancer in a patient.

According to a first aspect of the present invention there is provided a combination product comprising

a VEGFR tyrosine kinase inhibitor, or a pharmaceutically acceptable salt thereof, and

a mTOR-selective kinase inhibitor, or a pharmaceutically acceptable salt thereof, in association with a pharmaceutically acceptable adjuvant, diluent or carrier.

According to another aspect of the present invention there is provided a combination product comprising

a VEGFR tyrosine kinase inhibitor, or a pharmaceutically acceptable salt thereof, and

a mTOR-selective kinase inhibitor, or a pharmaceutically acceptable salt thereof, in association with a pharmaceutically acceptable adjuvant, diluent or carrier,

wherein the VEGFR tyrosine kinase inhibitor is 4-(4-bromo-2-fluoroanilino)-6-methoxy-7-(1-methylpiperidin-4-ylmethoxy)quinazoline or 4-(4-fluoro-2-methyl-1H-indol-5-yloxy)-6-methoxy-7-(3-(pyrrolidin-1-yl)propoxy)quinazoline, or a pharmaceutically acceptable salt thereof.

According to a further aspect of the present invention there is provided a combination product comprising

4-(4-fluoro-2-methyl-1H-indol-5-yloxy)-6-methoxy-7-(3-(pyrrolidin-1-yl)propoxy)quinazoline, or a pharmaceutically acceptable salt thereof, and

a mTOR-selective kinase inhibitor, or a pharmaceutically acceptable salt thereof, in association with a pharmaceutically acceptable adjuvant, diluent or carrier.

According to a further aspect of the present invention there is provided a combination product comprising

4-(4-bromo-2-fluoroanilino)-6-methoxy-7-(1-methylpiperidin-4-ylmethoxy)quinazoline, or a pharmaceutically acceptable salt thereof, and

a mTOR-selective kinase inhibitor, or a pharmaceutically acceptable salt thereof, in association with a pharmaceutically acceptable adjuvant, diluent or carrier.

The combination product of the present invention provides for the administration of 4-(4-fluoro-2-methyl-1H-indol-5-yloxy)-6-methoxy-7-(3-(pyrrolidin-1-yl)propoxy)quinazoline, or a pharmaceutically acceptable salt thereof, in conjunction with a mTOR-selective kinase inhibitor. The combination product, as defined herein, may be in the form of a combined preparation of 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor. The combination product, as defined herein, may comprise a kit of parts comprising separate formulations of 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor. The separate formulations of 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor may be administered sequentially, separately and/or simultaneously. In one embodiment the separate formulations of 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor of the combination product, as defined herein, are administered simultaneously (optionally repeatedly). In one embodiment the separate formulations of 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor of the combination product, as defined herein, are administered sequentially (optionally repeatedly). In one embodiment the separate formulations of 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor of the combination product, as defined herein, are administered separately (optionally repeatedly). The skilled person will understand that where the separate formulations of 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor of the combination product, as defined herein, are administered sequentially or serially that this could be administration of 4-(4-fluoro-2-methyl-1H-indol-5-yloxy)-6-methoxy-7-(3-(pyrrolidin-1-yl)propoxy)quinazoline, or a pharmaceutically acceptable salt thereof, followed by a mTOR-selective kinase inhibitor, or a mTOR-selective kinase inhibitor followed by 4-(4-fluoro-2-methyl-1H-indol-5-yloxy)-6-methoxy-7-(3-(pyrrolidin-1-yl)propoxy)quinazoline. Where the administration of the separate formulations of 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor of the combination product, as defined herein, is sequential or separate, the delay in administering the second formulation should not be such as to lose the beneficial effect of the combination therapy. Thus, the present invention provides a combination product, as defined herein, comprising 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor, or a pharmaceutically-acceptable salt thereof, for use sequentially, separately and/or simultaneously in the treatment of cancer.

The combination product of the present invention provides for the administration of 4-(4-bromo-2-fluoroanilino)-6-methoxy-7-(1-methylpiperidin-4-ylmethoxy)quinazoline, or a pharmaceutically acceptable salt thereof, in conjunction with a mTOR-selective kinase inhibitor. The combination product, as defined herein, may be in the form of a combined preparation of 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor. The combination product, as defined herein, may comprise a kit of parts comprising separate formulations of 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor. The separate formulations of 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor may be administered sequentially, separately and/or simultaneously. In one embodiment the separate formulations of 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor of the combination product, as defined herein, are administered simultaneously (optionally repeatedly). In one embodiment the separate formulations of 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor of the combination product, as defined herein, are administered sequentially (optionally repeatedly). In one embodiment the separate formulations of 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor of the combination product, as defined herein, are administered separately (optionally repeatedly). The skilled person will understand that where the separate formulations of 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor of the combination product, as defined herein, are administered sequentially or serially that this could be administration of 4-(4-bromo-2-fluoroanilino)-6-methoxy-7-(1-methylpiperidin-4-ylmethoxy)quinazoline, or a pharmaceutically acceptable salt thereof, followed by a mTOR-selective kinase inhibitor, or a mTOR-selective kinase inhibitor followed by 4-(4-bromo-2-fluoroanilino)-6-methoxy-7-(1-methylpiperidin-4-ylmethoxy)quinazoline. Where the administration of the separate formulations of 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor of the combination product, as defined herein, is sequential or separate, the delay in administering the second formulation should not be such as to lose the beneficial effect of the combination therapy. Thus, the present invention provides a combination product, as defined herein, comprising 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor, or a pharmaceutically-acceptable salt thereof, for use sequentially, separately and/or simultaneously in the treatment of cancer.

In another aspect there is provided a combination product, as defined herein, which comprises a kit of parts comprising the following components:
4-(4-fluoro-2-methyl-1H-indol-5-yloxy)-6-methoxy-7-(3-(pyrrolidin-1yl)propoxy)quinazoline, or a pharmaceutically acceptable salt thereof, in association with a pharmaceutically acceptable adjuvant, diluent or carrier; and
a mTOR-selective kinase inhibitor, or a pharmaceutically acceptable salt thereof, in association with a pharmaceutically acceptable adjuvant, diluent or carrier,
   wherein the components are provided in a form which is suitable for sequential, separate and/or simultaneous administration.

In one embodiment the kit of parts comprises
a first container comprising 4-(4-fluoro-2-methyl-1H-indol-5-yloxy)-6-methoxy-7-(3-(pyrrolidin-1-yl)propoxy)quinazoline, or a pharmaceutically acceptable salt thereof, in association with a pharmaceutically acceptable adjuvant, diluent or carrier; and
a second container comprising a mTOR-selective kinase inhibitor, or a pharmaceutically acceptable salt thereof, in association with a pharmaceutically acceptable adjuvant, diluent or carrier, and
   a container means for containing said first and second containers.

In one embodiment the kit of parts further comprises instructions to administer the components sequentially, separately and/or simultaneously. In one embodiment the kit of parts further comprises instructions indicating that the combination product, as defined herein, can be used in the treatment of cancer.

In another aspect there is provided a combination product, as defined herein, which comprises a kit of parts comprising the following components:
4-(4-bromo-2-fluoroanilino)-6-methoxy-7-(1-methylpiperidin-4-ylmethoxy)quinazoline, or a pharmaceutically acceptable salt thereof, in association with a pharmaceutically acceptable adjuvant, diluent or carrier; and
a mTOR-selective kinase inhibitor, or a pharmaceutically acceptable salt thereof, in association with a pharmaceutically acceptable adjuvant, diluent or carrier,
   wherein the components are provided in a form which is suitable for sequential, separate and/or simultaneous administration.

In one embodiment the kit of parts comprises
a first container comprising 4-(4-bromo-2-fluoroanilino)-6-methoxy-7-(1-methylpiperidin-4-ylmethoxy)quinazoline, or a pharmaceutically acceptable salt thereof, in association with a pharmaceutically acceptable adjuvant, diluent or carrier; and
a second container comprising a mTOR-selective kinase inhibitor, or a pharmaceutically acceptable salt thereof, in association with a pharmaceutically acceptable adjuvant, diluent or carrier, and
   a container means for containing said first and second containers.

In one embodiment the kit of parts further comprises instructions to administer the components sequentially, separately and/or simultaneously. In one embodiment the kit of parts further comprises instructions indicating that the combination product, as defined herein, can be used in the treatment of cancer.

In another aspect there is provided a combination product, as defined herein, comprising a pharmaceutical composition which comprises a VEGFR tyrosine kinase inhibitor, or a pharmaceutically acceptable salt thereof, and a mTOR-selective kinase inhibitor, or a pharmaceutically-acceptable salt thereof.

In another aspect there is provided a combination product, as defined herein, comprising a pharmaceutical composition which comprises 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor, or a pharmaceutically-acceptable salt thereof.

In another aspect there is provided a combination product, as defined herein, comprising a pharmaceutical composition which comprises 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor, or a pharmaceutically-acceptable salt thereof.

In another aspect there is provided a pharmaceutical composition which comprises a VEGFR tyrosine kinase inhibitor, or a pharmaceutically acceptable salt thereof, and a mTOR-selective kinase inhibitor, or a pharmaceutically-acceptable salt thereof.

In another aspect there is provided a pharmaceutical composition which comprises 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor, or a pharmaceutically-acceptable salt thereof.

In another aspect there is provided a pharmaceutical composition which comprises 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor, or a pharmaceutically-acceptable salt thereof.

According to the present invention there is provided a method for the production of an antiangiogenic and/or vascular permeability reducing effect in a warm-blooded animal such as a human, which comprises administering to said animal an effective amount of a VEGFR tyrosine kinase inhibitor, or a pharmaceutically acceptable salt thereof, before, after or simultaneously with an effective amount of a mTOR-selective kinase inhibitor, or a pharmaceutically-acceptable salt thereof.

According to the present invention there is provided a method for the production of an antiangiogenic and/or vascular permeability reducing effect in a warm-blooded animal such as a human, which comprises administering to said animal an effective amount of 4-(4-fluoro-2-methyl-1H-indol-5-yloxy)-6-methoxy-7-(3-(pyrrolidin-1-yl)propoxy)quinazoline, or a pharmaceutically acceptable salt thereof, before, after or simultaneously with an effective amount of a mTOR-selective kinase inhibitor, or a pharmaceutically-acceptable salt thereof.

According to the present invention there is provided a method for the production of an antiangiogenic and/or vascular permeability reducing effect in a warm-blooded animal such as a human, which comprises administering to said animal an effective amount of 4-(4-bromo-2-fluoroanilino)-6-methoxy-7-(1-methylpiperidin-4-ylmethoxy)quinazoline, or a pharmaceutically acceptable salt thereof, before, after or simultaneously with an effective amount of a mTOR-selective kinase inhibitor, or a pharmaceutically-acceptable salt thereof.

According to a further aspect of the present invention there is provided a method for the treatment of a cancer in a warm-blooded animal such as a human, which comprises administering to said animal an effective amount of a VEGFR tyrosine kinase inhibitor, or a pharmaceutically acceptable salt thereof, before, after or simultaneously with an effective amount of a mTOR-selective kinase inhibitor, or a pharmaceutically-acceptable salt thereof.

According to a further aspect of the present invention there is provided a method for the treatment of a cancer in a warm-blooded animal such as a human, which comprises administering to said animal an effective amount of 4-(4-fluoro-2-methyl-1H-indol-5-yloxy)-6-methoxy-7-(3-(pyrrolidin-1-yl)propoxy)quinazoline, or a pharmaceutically acceptable salt thereof, before, after or simultaneously with an effective amount of a mTOR-selective kinase inhibitor, or a pharmaceutically-acceptable salt thereof.

According to a further aspect of the present invention there is provided a method for the treatment of a cancer in a warm-blooded animal such as a human, which comprises administering to said animal an effective amount of 4-(4-bromo-2-fluoroanilino)-6-methoxy-7-(1-methylpiperidin-4-ylmethoxy)quinazoline, or a pharmaceutically acceptable salt thereof, before, after or simultaneously with an effective amount of a mTOR-selective kinase inhibitor, or a pharmaceutically-acceptable salt thereof.

According to a further aspect of the present invention there is provided a method for the treatment of a cancer involving a solid tumour in a warm-blooded animal such as a human, which comprises administering to said animal an effective amount of a VEGFR tyrosine kinase inhibitor, or a pharmaceutically acceptable salt thereof, before, after or simultaneously with an effective amount of a mTOR-selective kinase inhibitor, or a pharmaceutically-acceptable salt thereof.

According to a further aspect of the present invention there is provided a method for the treatment of a cancer involving a solid tumour in a warm-blooded animal such as a human, which comprises administering to said animal an effective amount of 4-(4-fluoro-2-methyl-1H-indol-5-yloxy)-6-methoxy-7-(3-(pyrrolidin-1-yl)propoxy)quinazoline, or a pharmaceutically acceptable salt thereof, before, after or simultaneously with an effective amount of a mTOR-selective kinase inhibitor, or a pharmaceutically-acceptable salt thereof.

According to a further aspect of the present invention there is provided a method for the treatment of a cancer involving a solid tumour in a warm-blooded animal such as a human, which comprises administering to said animal an effective amount of 4-(4-bromo-2-fluoroanilino)-6-methoxy-7-(1-methylpiperidin-4-ylmethoxy)quinazoline, or a pharmaceutically acceptable salt thereof, before, after or simultaneously with an effective amount of a mTOR-selective kinase inhibitor, or a pharmaceutically-acceptable salt thereof.

According to a further aspect of the present invention there is provided a method for the production of an antiangiogenic and/or vascular permeability reducing effect in a warm-blooded animal such as a human, which comprises administering to said animal an effective amount of a VEGFR tyrosine kinase inhibitor, or a pharmaceutically acceptable salt thereof, before, after or simultaneously with an effective amount of a mTOR-selective kinase inhibitor, or a pharmaceutically-acceptable salt thereof; wherein a VEGFR tyrosine kinase inhibitor, or a pharmaceutically acceptable salt thereof, and the mTOR-selective kinase inhibitor may each optionally be administered together with a pharmaceutically acceptable excipient or carrier.

According to a further aspect of the present invention there is provided a method for the production of an antiangiogenic and/or vascular permeability reducing effect in a warm-blooded animal such as a human, which comprises administering to said animal an effective amount of 4-(4-fluoro-2-methyl-1H-indol-5-yloxy)-6-methoxy-7-(3-(pyrrolidin-1-yl)propoxy)quinazoline, or a pharmaceutically acceptable salt thereof, before, after or simultaneously with an effective amount of a mTOR-selective kinase inhibitor, or a pharmaceutically-acceptable salt thereof; wherein 4-(4-fluoro-2-methyl-1H-indol-5-yloxy)-6-methoxy-7-(3-(pyrrolidin-1-yl)propoxy)quinazoline, or a pharmaceutically acceptable salt thereof, and the mTOR-selective kinase inhibitor may each optionally be administered together with a pharmaceutically acceptable excipient or carrier.

According to a further aspect of the present invention there is provided a method for the production of an antiangiogenic and/or vascular permeability reducing effect in a warm-blooded animal such as a human, which comprises administering to said animal an effective amount of 4-(4-bromo-2-fluoroanilino)-6-methoxy-7-(1-methylpiperidin-4-ylmethoxy)quinazoline, or a pharmaceutically acceptable salt thereof, before, after or simultaneously with an effective amount of a mTOR-selective kinase inhibitor, or a pharmaceutically-acceptable salt thereof; wherein 4-(4-bromo-2-fluoroanilino)-6-methoxy-7-(1-methylpiperidin-4-ylmethoxy)quinazoline, or a pharmaceutically acceptable salt thereof, and the mTOR-selective kinase inhibitor may each optionally be administered together with a pharmaceutically acceptable excipient or carrier.

According to a further aspect of the present invention there is provided a method for the treatment of a cancer in a warm-blooded animal such as a human, which comprises administering to said animal an effective amount of a VEGFR tyrosine kinase inhibitor, or a pharmaceutically acceptable salt thereof, before, after or simultaneously with an effective amount of a mTOR-selective kinase inhibitor, or a pharmaceutically-acceptable salt thereof; wherein a VEGFR tyrosine kinase inhibitor, or a pharmaceutically acceptable salt thereof, and the mTOR-selective kinase inhibitor may each optionally be administered together with a pharmaceutically acceptable excipient or carrier.

According to a further aspect of the present invention there is provided a method for the treatment of a cancer in a warm-blooded animal such as a human, which comprises administering to said animal an effective amount of 4-(4-fluoro-2-methyl-1H-indol-5-yloxy)-6-methoxy-7-(3-(pyrrolidin-1-yl)propoxy)quinazoline, or a pharmaceutically acceptable salt thereof, before, after or simultaneously with an effective amount of a mTOR-selective kinase inhibitor, or a pharmaceutically-acceptable salt thereof; wherein 4-(4-fluoro-2-methyl-1H-indol-5-yloxy)-6-methoxy-7-(3-(pyrrolidin-1-yl)propoxy)quinazoline, or a pharmaceutically acceptable salt thereof, and the mTOR-selective kinase inhibitor may each optionally be administered together with a pharmaceutically acceptable excipient or carrier.

According to a further aspect of the present invention there is provided a method for the treatment of a cancer in a warm-blooded animal such as a human, which comprises administering to said animal an effective amount of 4-(4-bromo-2-fluoroanilino)-6-methoxy-7-(1-methylpiperidin-4-ylmethoxy)quinazoline, or a pharmaceutically acceptable salt thereof, before, after or simultaneously with an effective amount of a mTOR-selective kinase inhibitor, or a pharmaceutically-acceptable salt thereof; wherein 4-(4-bromo-2-fluoroanilino)-6-methoxy-7-(1-methylpiperidin-4-ylmethoxy)quinazoline, or a pharmaceutically acceptable salt thereof, and the mTOR-selective kinase inhibitor may each optionally be administered together with a pharmaceutically acceptable excipient or carrier.

According to a further aspect of the present invention there is provided a method for the treatment of a cancer involving a solid tumour in a warm-blooded animal such as a human, which comprises administering to said animal an effective amount of a VEGFR tyrosine kinase inhibitor, or a pharmaceutically acceptable salt thereof, before, after or simultaneously with an effective amount of a mTOR-selective kinase inhibitor, or a pharmaceutically-acceptable salt thereof; wherein the VEGFR tyrosine kinase inhibitor and the mTOR-selective kinase inhibitor may each optionally be administered together with a pharmaceutically acceptable excipient or carrier.

According to a further aspect of the present invention there is provided a method for the treatment of a cancer involving a solid tumour in a warm-blooded animal such as a human, which comprises administering to said animal an effective amount of 4-(4-fluoro-2-methyl-1H-indol-5-yloxy)-6-methoxy-7-(3-(pyrrolidin-1-yl)propoxy)quinazoline, or a pharmaceutically acceptable salt thereof, before, after or simultaneously with an effective amount of a mTOR-selective kinase inhibitor, or a pharmaceutically-acceptable salt thereof; wherein 4-(4-fluoro-2-methyl-1H-indol-5-yloxy)-6-methoxy-7-(3-(pyrrolidin-1-yl)propoxy)quinazoline and the mTOR-selective kinase inhibitor may each optionally be administered together with a pharmaceutically acceptable excipient or carrier.

According to a further aspect of the present invention there is provided a method for the treatment of a cancer involving a solid tumour in a warm-blooded animal such as a human, which comprises administering to said animal an effective amount of 4-(4-bromo-2-fluoroanilino)-6-methoxy-7-(1-methylpiperidin-4-ylmethoxy)quinazoline, or a pharmaceutically acceptable salt thereof, before, after or simultaneously with an effective amount of a mTOR-selective kinase inhibitor, or a pharmaceutically-acceptable salt thereof; wherein 4-(4-bromo-2-fluoroanilino)-6-methoxy-7-(1-methylpiperidin-4-ylmethoxy)quinazoline and the mTOR-selective kinase inhibitor may each optionally be administered together with a pharmaceutically acceptable excipient or carrier.

According to a further aspect of the invention there is provided a pharmaceutical composition which comprises a VEGFR tyrosine kinase inhibitor, or a pharmaceutically acceptable salt thereof, and a mTOR-selective kinase inhibitor, or a pharmaceutically-acceptable salt thereof, in association with a pharmaceutically acceptable excipient or carrier.

According to a further aspect of the invention there is provided a pharmaceutical composition which comprises 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor, or a pharmaceutically-acceptable salt thereof, in association with a pharmaceutically acceptable excipient or carrier.

According to a further aspect of the invention there is provided a pharmaceutical composition which comprises 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor, or a pharmaceutically-acceptable salt thereof, in association with a pharmaceutically acceptable excipient or carrier.

According to a further aspect of the present invention there is provided a combination product comprising a VEGFR tyrosine kinase inhibitor, or a pharmaceutically acceptable salt thereof, and a mTOR-selective kinase inhibitor, or a pharmaceutically-acceptable salt thereof, for use in a method of treatment of a human or animal body by therapy.

According to a further aspect of the present invention there is provided a combination product comprising 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor, or a pharmaceutically-acceptable salt thereof, for use in a method of treatment of a human or animal body by therapy.

According to a further aspect of the present invention there is provided a combination product comprising 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor, or a pharmaceutically-acceptable salt thereof, for use in a method of treatment of a human or animal body by therapy.

According to a further aspect of the present invention there is provided a kit comprising a VEGFR tyrosine kinase inhibitor, or a pharmaceutically acceptable salt thereof, and a mTOR-selective kinase inhibitor, or a pharmaceutically-acceptable salt thereof.

According to a further aspect of the present invention there is provided a kit comprising 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor, or a pharmaceutically-acceptable salt thereof.

According to a further aspect of the present invention there is provided a kit comprising:

a) 4-(4-fluoro-2-methyl-1H-indol-5-yloxy)-6-methoxy-7-(3-(pyrrolidin-1-yl)propoxy)quinazoline, or a pharmaceutically acceptable salt thereof, in a first unit dosage form;
b) a mTOR-selective kinase inhibitor or a pharmaceutically-acceptable salt thereof in a second unit dosage form; and
c) container means for containing said first and second dosage forms.

According to a further aspect of the present invention there is provided a kit comprising:

a) 4-(4-fluoro-2-methyl-1H-indol-5-yloxy)-6-methoxy-7-(3-(pyrrolidin-1-yl)propoxy)quinazoline, or a pharmaceutically acceptable salt thereof, together with a pharmaceutically acceptable excipient or carrier, in a first unit dosage form;
b) a mTOR-selective kinase inhibitor or a pharmaceutically-acceptable salt thereof together with a pharmaceutically acceptable excipient or carrier, in a second unit dosage form; and
c) container means for containing said first and second dosage forms.

According to a further aspect of the present invention there is provided a kit comprising 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor, or a pharmaceutically-acceptable salt thereof.

According to a further aspect of the present invention there is provided a kit comprising:

a) 4-(4-bromo-2-fluoroanilino)-6-methoxy-7-(1-methylpiperidin-4-ylmethoxy)quinazoline, or a pharmaceutically acceptable salt thereof, in a first unit dosage form;
b) a mTOR-selective kinase inhibitor or a pharmaceutically-acceptable salt thereof in a second unit dosage form; and
c) container means for containing said first and second dosage forms.

According to a further aspect of the present invention there is provided a kit comprising:

a) 4-(4-bromo-2-fluoroanilino)-6-methoxy-7-(1-methylpiperidin-4-ylmethoxy)quinazoline, or a pharmaceutically acceptable salt thereof, together with a pharmaceutically acceptable excipient or carrier, in a first unit dosage form;
b) a mTOR-selective kinase inhibitor or a pharmaceutically-acceptable salt thereof together with a pharmaceutically acceptable excipient or carrier, in a second unit dosage form; and
c) container means for containing said first and second dosage forms.

According to a further aspect of the present invention there is provided the use of a VEGFR tyrosine kinase inhibitor, or a pharmaceutically acceptable salt thereof, and a mTOR-selective kinase inhibitor, or a pharmaceutically-acceptable salt thereof, in the manufacture of a medicament for use in the production of an antiangiogenic and/or vascular permeability reducing effect in a warm-blooded animal such as a human.

According to a further aspect of the present invention there is provided the use of 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor, or a pharmaceutically-acceptable salt thereof, in the manufacture of a medicament for use in the production of an antiangiogenic and/or vascular permeability reducing effect in a warm-blooded animal such as a human.

According to a further aspect of the present invention there is provided the use of 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor, or a pharmaceutically-acceptable salt thereof, in the manufacture of a medicament for use in the production of an antiangiogenic and/or vascular permeability reducing effect in a warm-blooded animal such as a human.

According to a further aspect of the present invention there is provided the use of a VEGFR tyrosine kinase inhibitor, or a pharmaceutically acceptable salt thereof, and a mTOR-selective kinase inhibitor, or a pharmaceutically-acceptable salt thereof, in the manufacture of a medicament for use in the production of an anti-cancer effect in a warm-blooded animal such as a human.

According to a further aspect of the present invention there is provided the use of 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor, or a pharmaceutically-acceptable salt thereof, in the manufacture of a medicament for use in the production of an anti-cancer effect in a warm-blooded animal such as a human.

According to a further aspect of the present invention there is provided the use of 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor, or a pharmaceutically-acceptable salt thereof, in the manufacture of a medicament for use in the production of an anti-cancer effect in a warm-blooded animal such as a human.

According to a further aspect of the present invention there is provided the use of a VEGFR tyrosine kinase inhibitor, or a pharmaceutically acceptable salt thereof, and a mTOR-selective kinase inhibitor or a pharmaceutically-acceptable salt thereof, in the manufacture of a medicament for use in the production of an anti-tumour effect in a warm-blooded animal such as a human.

According to a further aspect of the present invention there is provided the use of 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor or a pharmaceutically-acceptable salt thereof, in the manufacture of a medicament for use in the production of an anti-tumour effect in a warm-blooded animal such as a human.

According to a further aspect of the present invention there is provided the use of 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor or a pharmaceutically-acceptable salt thereof, in the manufacture of a medicament for use in the production of an anti-tumour effect in a warm-blooded animal such as a human.

According to a further aspect of the present invention there is provided a combination treatment comprising the administration of an effective amount of a VEGFR tyrosine kinase inhibitor, or a pharmaceutically acceptable salt thereof, optionally together with a pharmaceutically acceptable excipient or carrier, and the simultaneous, sequential or separate administration of an effective amount of a mTOR-selective kinase inhibitor, or a pharmaceutically-acceptable salt thereof; wherein the mTOR-selective kinase inhibitor may optionally be administered together with a pharmaceutically acceptable excipient or carrier; to a warm-blooded animal such as a human in need of such therapeutic treatment.

According to a further aspect of the present invention there is provided a combination treatment comprising the administration of an effective amount of 4-(4-fluoro-2-methyl-1H-indol-5-yloxy)-6-methoxy-7-(3-(pyrrolidin-1-yl)propoxy)quinazoline, or a pharmaceutically acceptable salt thereof, optionally together with a pharmaceutically acceptable excipient or carrier, and the simultaneous, sequential or separate administration of an effective amount of a mTOR-selective kinase inhibitor, or a pharmaceutically-acceptable salt thereof; wherein the mTOR-selective kinase inhibitor may optionally be administered together with a pharmaceutically acceptable excipient or carrier; to a warm-blooded animal such as a human in need of such therapeutic treatment.

According to a further aspect of the present invention there is provided a combination treatment comprising the administration of an effective amount of 4-(4-bromo-2-fluoroanilino)-6-methoxy-7-(1-methylpiperidin-4-ylmethoxy)quinazoline, or a pharmaceutically acceptable salt thereof, optionally together with a pharmaceutically acceptable excipient or carrier, and the simultaneous, sequential or separate administration of an effective amount of a mTOR-selective kinase inhibitor, or a pharmaceutically-acceptable salt thereof; wherein the mTOR-selective kinase inhibitor may optionally be administered together with a pharmaceutically acceptable excipient or carrier; to a warm-blooded animal such as a human in need of such therapeutic treatment.

Therapeutic treatment includes an antiangiogenic and/or vascular permeability effect, an anti-tumorigenic effect, an anti-cancer effect and an anti-tumour effect.

The combination product of the present invention and the methods of treatment comprising the administering or use of 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor, or a pharmaceutically acceptable salt thereof, are of interest for their antiangiogenic and/or vascular permeability effects.

The combination product and the methods of treatment comprising the administering or use of 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor, or a pharmaceutically acceptable salt thereof, are also of interest for their antiangiogenic and/or vascular permeability effects.

Angiogenesis and/or an increase in vascular permeability is present in a wide range of disease states including cancer (including leukaemia, multiple myeloma and lymphoma), diabetes, psoriasis, rheumatoid arthritis, Kaposi's sarcoma, haemangioma, acute and chronic nephropathies, atheroma, arterial restenosis, autoimmune diseases, acute inflammation, asthma, lymphoedema, endometriosis, dysfunctional uterine bleeding and ocular diseases with retinal vessel proliferation including age-related macular degeneration.

Anti-cancer effects which are accordingly useful in the treatment of cancer in a patient include, but are not limited to, anti-tumour effects, the response rate, the time to disease progression and the survival rate. Anti-tumour effects of a method of treatment of the present invention include but are not limited to, inhibition of tumour growth, tumour growth delay, regression of tumour, shrinkage of tumour, increased time to regrowth of tumour on cessation of treatment, slowing of disease progression. It is expected that when a combination product of the present invention is administered to a patient in need of treatment for cancer, said combination product, as defined herein, will produce an effect, as measured by, for example, one or more of: the extent of the anti-tumour effect, the response rate, the time to disease progression and the survival rate. Anti-cancer effects include prophylactic treatment as well as treatment of existing disease.

The combination product of the present invention and the methods of treatment comprising the administering or use of 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor, or a pharmaceutically acceptable salt thereof, are expected to be particularly useful for the treatment patients with cancers, including, but not limited to, haematologic malignancies such as leukaemia, multiple myeloma, lymphomas such as Hodgkin's disease, non-Hodgkin's lymphomas (including mantle cell lymphoma), and myelodysplastic syndromes, and also solid tumours and their metastases such as breast cancer, lung cancer (non-small cell lung cancer (NSCL), small cell lung cancer (SCLC), squamous cell carcinoma), endometrial cancer, tumours of the central nervous system such as gliomas, dysembryoplastic neuroepithelial tumour, glioblastoma multiforme, mixed gliomas, medulloblastoma, retinoblastoma, neuroblastoma, germinoma and teratoma, cancers of the gastrointestinal tract such as gastric cancer, oesophagal cancer, hepatocellular (liver) carcinoma, cholangiocarcinomas, colon and rectal carcinomas, cancers of the small intestine, pancreatic cancers, cancers of the skin such as melanomas (in particular metastatic melanoma), thyroid cancers, cancers of the head and neck and cancers of the salivary glands, prostate, testis, ovary, cervix, uterus, vulva, bladder, kidney (including renal cell carcinoma, clear cell and renal oncocytoma), squamous cell carcinomas, sarcomas such as osteosarcoma, chondrosarcoma, leiomyosarcoma, soft tissue sarcoma, Ewing's sarcoma, gastrointestinal stromal tumour (GIST), Kaposi's sarcoma, and paediatric cancers such as rhabdomyosarcomas and neuroblastomas.

The combination product of the present invention and the methods of treatment comprising the administering or use of 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor, or a pharmaceutically acceptable salt thereof, are expected to be particularly useful for the treatment patients with lung cancer, prostate cancer, melanoma, ovarian cancer, breast cancer, endometrial cancer, kidney cancer, gastric cancer, sarcomas, head and neck cancers, tumours of the central nervous system and their metastases, and also for the treatment of patients with acute myeloid leukaemia.

The combination product of the present invention and the methods of treatment comprising the administering or use of 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor, or a pharmaceutically acceptable salt thereof, are expected to be particularly useful for the treatment patients with lung cancer, prostate cancer, melanoma, ovarian cancer, breast cancer, endometrial cancer, kidney cancer, gastric cancer, sarcomas, head and neck cancers, tumours of the central nervous system and their metastases, and also for the treatment of patients with acute myeloid leukaemia.

The combination product of the present invention and the methods of treatment comprising the administering or use of 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor, or a pharmaceutically acceptable salt thereof, are also expected to be particularly useful for the treatment of patients with a tumour which is ameliorated by the inhibition of mTOR.

The combination product of the present invention and the methods of treatment comprising the administering or use of 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor, or a pharmaceutically acceptable salt thereof, are also expected to be particularly useful for the treatment of patients with a tumour which is ameliorated by the inhibition of mTOR.

The combination product of the present invention and the methods of treatment comprising the administering or use of 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor, or a pharmaceutically acceptable salt thereof, are also expected to be particularly useful for the treatment of patients with a tumour which is associated with VEGF or which is dependent alone, or in part, on the biological activity of VEGF.

The combination product of the present invention and the methods of treatment comprising the administering or use of 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor, or a pharmaceutically acceptable salt thereof, are also expected to be particularly useful for the treatment of patients with a tumour which is associated with VEGF or which is dependent alone, or in part, on the biological activity of VEGF.

The combination product of the present invention and the methods of treatment comprising the administering or use of 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor, or a pharmaceutically acceptable salt thereof, are also expected to be particularly useful for the treatment of patients with a tumour which is associated with the PI3K/AKT pathway or which is dependent alone, or in part, on the biological activity of the PI3K/AKT pathway.

The combination product of the present invention and the methods of treatment comprising the administering or use of 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor, or a pharmaceutically acceptable salt thereof, are also expected to be particularly useful for the treatment of patients with a tumour which is associated with the PI3K/AKT pathway or which is dependent alone, or in part, on the biological activity of the PI3K/AKT pathway.

The combination product of the present invention and the methods of treatment comprising the administering or use of 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor, or a pharmaceutically acceptable salt thereof, are also expected to be particularly useful for the treatment of patients with a tumour which is associated with mTOR or which is dependent alone, or in part, on the biological activity of mTOR.

The combination product of the present invention and the methods of treatment comprising the administering or use of 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor, or a pharmaceutically acceptable salt thereof, are also expected to be particularly useful for the treatment of patients with a tumour which is associated with mTOR or which is dependent alone, or in part, on the biological activity of mTOR.

The combination product of the present invention and the methods of treatment comprising the administering or use of 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor, or a pharmaceutically acceptable salt thereof, are expected to produce a synergistic or beneficial effect through the production of an anti-cancer effect in a patient, which is accordingly useful in the treatment of cancer in a patient. A beneficial effect is achieved if the effect is therapeutically superior, as measured by, for example, the extent of the response, the response rate, the time to onset of disease, the time to disease progression or the survival period, to that achievable on dosing one or other of the components of the combination treatment at its conventional dose. The beneficial effect may be synergistic, if the combined effect is therapeutically superior to the sum of the individual effect achievable with 4-(4-fluoro-2-methyl-1H-indol-5-yloxy)-6-methoxy-7-(3-(pyrrolidin-1-yl)propoxy)quinazoline or a mTOR-selective kinase inhibitor. Further, a beneficial effect is obtained if an effect is achieved in a group of patients that does not respond (or responds poorly) to an antagonist of the biological activity of 4-(4-fluoro-2-methyl-1H-indol-5-yloxy)-6-methoxy-7-(3-(pyrrolidin-1-yl)propoxy)quinazoline or a mTOR-selective kinase inhibitor alone. In addition, the effect is defined as affording a beneficial effect if one of the components is dosed at its conventional dose and the other component(s) is/are dosed at a reduced dose and the therapeutic effect, as measured by, for example, the extent of the response, the response rate, the time to onset of disease, the time to disease progression or the survival period, is equivalent to that achievable on dosing conventional amounts of the components of the combination treatment. In particular, a beneficial effect is deemed to be achieved if a conventional dose of 4-(4-fluoro-2-methyl-1H-indol-5-yloxy)-6-methoxy-7-(3-(pyrrolidin-1-yl)propoxy)quinazoline or a mTOR-selective kinase inhibitor may be reduced without detriment to one or more of the extent of the response, the response rate, the time to onset of disease, the time to disease progression and survival data, in particular without detriment to the duration of the response, but with fewer and/or less troublesome side-effects than those that occur when conventional doses of each component are used.

The combination product and the methods of treatment comprising the administering or use of 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor, or a pharmaceutically acceptable salt thereof, are expected to produce a synergistic or beneficial effect through the production of an anti-cancer effect in a patient, which is accordingly useful in the treatment of cancer in a patient. A beneficial effect is achieved if the effect is therapeutically superior, as measured by, for example, the extent of the response, the response rate, the time to onset of disease, the time to disease progression or the survival period, to that achievable on dosing one or other of the components of the combination treatment at its conventional dose. The beneficial effect may be synergistic, if the combined effect is therapeutically superior to the sum of the individual effect achievable with 4-(4-bromo-2-fluoroanilino)-6-methoxy-7-(1-methylpiperidin-4-ylmethoxy)quinazoline or a mTOR-selective kinase inhibitor. Further, a beneficial effect is obtained if an effect is achieved in a group of patients that does not respond (or responds poorly) to an antagonist of the biological activity of 4-(4-bromo-2-fluoroanilino)-6-methoxy-7-(1-methylpiperidin-4-ylmethoxy)quinazoline or a mTOR-selective kinase inhibitor alone. In addition, the effect is defined as affording a beneficial effect if one of the components is dosed at its conventional dose and the other component(s) is/are dosed at a reduced dose and the therapeutic effect, as measured by, for example, the extent of the response, the response rate, the time to onset of disease, the time to disease progression or the survival period, is equivalent to that achievable on dosing conventional amounts of the components of the combination treatment. In particular, a beneficial effect is deemed to be achieved if a conventional dose of 4-(4-bromo-2-fluoroanilino)-6-methoxy-7-(1-methylpiperidin-4-ylmethoxy)quinazoline or a mTOR-selective kinase inhibitor may be reduced without detriment to one or more of the extent of the response, the response rate, the time to onset of disease, the time to disease progression and survival data, in particular without detriment to the duration of the response, but with fewer and/or less troublesome side-effects than those that occur when conventional doses of each component are used.

In another aspect of the present invention there is provided a method for producing immunosuppression, immune-tolerance or for treating autoimmune disease, inflammation, bone loss, bowel disorders, hepatic fibrosis, hepatic necrosis, rheumatoid arthritis, restenosis, cardiac allograft vasculopathy, psoriasis, beta-thalassaemia, fungal infections and ocular conditions such as dry eye, age related macular degeneration, glaucoma and uveitis, which comprises administration of a therapeutically effective amount of a combination product, as defined herein, of the invention to a patient having or suspected of having any one or more of the above conditions. In one embodiment 4-(4-fluoro-2-methyl-1H-indol-5-yloxy)-6-methoxy-7-(3-(pyrrolidin-1-yl)propoxy)quinazoline, or a pharmaceutically acceptable salt thereof, is administered sequentially, separately and/or simultaneously with the mTOR-selective kinase inhibitor, or a pharmaceutically acceptable salt thereof. In one embodiment 4-(4-bromo-2-fluoroanilino)-6-methoxy-7-(1-methylpiperidin-4-ylmethoxy)quinazoline, or a pharmaceutically acceptable salt thereof, is administered sequentially, separately and/or simultaneously with the mTOR-selective kinase inhibitor, or a pharmaceutically acceptable salt thereof. In one embodiment the method additionally comprises selecting a patient in need of treatment for one or more of the above conditions, and administration to the patient of a therapeutically effective dose of a combination product, as defined herein, of the invention. Such methods of treatment for one or more of the above conditions may also give rise to synergistic or beneficial effects.

A combination treatment of the present invention as defined herein may be achieved by way of the simultaneous, sequential or separate administration of the individual components of said treatment. A combination treatment as defined herein may be applied as a sole therapy or may involve surgery or radiotherapy or an additional chemotherapeutic agent in addition to a combination treatment of the invention. Surgery may comprise the step of partial or complete tumour resection, prior to, during or after the administration of the combination treatment with a VEGFR tyrosine kinase inhibitor, or a pharmaceutically acceptable salt thereof, and a mTOR-selective kinase inhibitor, or a pharmaceutically acceptable salt thereof, described herein.

Surgery may comprise the step of partial or complete tumour resection, prior to, during or after the administration of the combination treatment with 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor, or a pharmaceutically acceptable salt thereof, described herein.

Surgery may comprise the step of partial or complete tumour resection, prior to, during or after the administration of the combination treatment with 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor, or a pharmaceutically acceptable salt thereof, described herein.

The administration of a triple combination of a VEGFR tyrosine kinase inhibitor, or a pharmaceutically acceptable salt thereof, a mTOR-selective kinase inhibitor, or a pharmaceutically acceptable salt thereof, and ionising radiation may produce effects, such as anti-tumour effects, greater than those achieved with any of a VEGFR tyrosine kinase inhibitor, or a pharmaceutically acceptable salt thereof, a mTOR-selective kinase inhibitor, or a pharmaceutically acceptable salt thereof, and ionising radiation used alone, greater than those achieved with the combination of a VEGFR tyrosine kinase inhibitor, or a pharmaceutically acceptable salt thereof, and a mTOR-selective kinase inhibitor, or a pharmaceutically acceptable salt thereof, greater than those achieved with the combination of a VEGFR tyrosine kinase inhibitor, or a pharmaceutically acceptable salt thereof, and ionising radiation, greater than those achieved with the combination of a mTOR-selective kinase inhibitor, or a pharmaceutically acceptable salt thereof, and ionising radiation.

The administration of a triple combination of 4-(4-fluoro-2-methyl-1H-indol-5-yloxy)-6-methoxy-7-(3-(pyrrolidin-1-yl)propoxy)quinazoline, or a pharmaceutically acceptable salt thereof, a mTOR-selective kinase inhibitor, or a pharmaceutically acceptable salt thereof, and ionising radiation may produce effects, such as anti-tumour effects, greater than those achieved with any of 4-(4-fluoro-2-methyl-1H-indol-5-yloxy)-6-methoxy-7-(3-(pyrrolidin-1-yl)propoxy)quinazoline, or a pharmaceutically acceptable salt thereof, a mTOR-selective kinase inhibitor, or a pharmaceutically acceptable salt thereof, and ionising radiation used alone, greater than those achieved with the combination of 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor, or a pharmaceutically acceptable salt thereof, greater than those achieved with the combination of 4-(4-fluoro-2-methyl-1H-indol-5-yloxy)-6-methoxy-7-(3-(pyrrolidin-1-yl)propoxy)quinazoline, or a pharmaceutically acceptable salt thereof, and ionising radiation, greater than those achieved with the combination of a mTOR-selective kinase inhibitor, or a pharmaceutically acceptable salt thereof, and ionising radiation.

According to the present invention there is provided a method for the production of an antiangiogenic and/or vascular permeability reducing effect in a warm-blooded animal such as a human, which comprises administering to said animal an effective amount of 4-(4-fluoro-2-methyl-1H-indol-5-yloxy)-6-methoxy-7-(3-(pyrrolidin-1-yl)propoxy)quinazoline, or a pharmaceutically acceptable salt thereof, before, after or simultaneously with an effective amount of a mTOR-selective kinase inhibitor or a pharmaceutically acceptable salt thereof, and before, after or simultaneously with an effective amount of ionising radiation.

According to a further aspect of the present invention there is provided a method for the treatment of a cancer in a warm-blooded animal such as a human, which comprises administering to said animal an effective amount of 4-(4-fluoro-2-methyl-1H-indol-5-yloxy)-6-methoxy-7-(3-(pyrrolidin-1-yl)propoxy)quinazoline, or a pharmaceutically acceptable salt thereof, before, after or simultaneously with an effective amount of a mTOR-selective kinase inhibitor or a pharmaceutically acceptable salt thereof, and before, after or simultaneously with an effective amount of ionising radiation.

According to a further aspect of the present invention there is provided a method for the treatment of a cancer involving a solid tumour in a warm-blooded animal such as a human, which comprises administering to said animal an effective amount of 4-(4-fluoro-2-methyl-1H-indol-5-yloxy)-6-methoxy-7-(3-(pyrrolidin-1-yl)propoxy)quinazoline, or a pharmaceutically acceptable salt thereof, before, after or simultaneously with an effective amount of a mTOR-selective kinase inhibitor or a pharmaceutically acceptable salt thereof, and before, after or simultaneously with an effective amount of ionising radiation.

According to a further aspect of the present invention there is provided a method for the production of an antiangiogenic and/or vascular permeability reducing effect in a warm-blooded animal such as a human, which comprises administering to said animal an effective amount of 4-(4-fluoro-2-methyl-1H-indol-5-yloxy)-6-methoxy-7-(3-(pyrrolidin-1-yl)propoxy)quinazoline, or a pharmaceutically acceptable salt thereof, before, after or simultaneously with an effective amount of a mTOR-selective kinase inhibitor or a pharmaceutically acceptable salt thereof, and before, after or simultaneously with an effective amount of ionising radiation, wherein 4-(4-fluoro-2-methyl-1H-indol-5-yloxy)-6-methoxy-7-(3-(pyrrolidin-1-yl)propoxy)quinazoline, or a pharmaceutically acceptable salt thereof, and the mTOR-selective kinase inhibitor may each optionally be administered together with a pharmaceutically acceptable excipient or carrier.

According to a further aspect of the present invention there is provided a method for the treatment of a cancer in a warm-blooded animal such as a human, which comprises administering to said animal an effective amount of 4-(4-fluoro-2-methyl-1H-indol-5-yloxy)-6-methoxy-7-(3-(pyrrolidin-1-yl)propoxy)quinazoline, or a pharmaceutically acceptable salt thereof, before, after or simultaneously with an effective amount of a mTOR-selective kinase inhibitor or a pharmaceutically acceptable salt thereof, and before, after or simultaneously with an effective amount of ionising radiation, wherein 4-(4-fluoro-2-methyl-1H-indol-5-yloxy)-6-methoxy-7-(3-(pyrrolidin-1-yl)propoxy)quinazoline, or a pharmaceutically acceptable salt thereof, and the mTOR-selective kinase inhibitor may each optionally be administered together with a pharmaceutically acceptable excipient or carrier.

According to a further aspect of the present invention there is provided a method for the treatment of a cancer involving a solid tumour in a warm-blooded animal such as a human, which comprises administering to said animal an effective amount of 4-(4-fluoro-2-methyl-1H-indol-5-yloxy)-6-methoxy-7-(3-(pyrrolidin-1-yl)propoxy)quinazoline, or a pharmaceutically acceptable salt thereof, before, after or simultaneously with an effective amount of a mTOR-selective kinase inhibitor or a pharmaceutically acceptable salt thereof, and before, after or simultaneously with an effective amount of ionising radiation, wherein 4-(4-fluoro-2-methyl-1H-indol-5-yloxy)-6-methoxy-7-(3-(pyrrolidin-1-yl)propoxy)quinazoline, or a pharmaceutically acceptable salt thereof, and the mTOR-selective kinase inhibitor may each optionally be administered together with a pharmaceutically acceptable excipient or carrier.

According to a further aspect of the present invention there is provided the use of 4-(4-fluoro-2-methyl-1H-indol-5-yloxy)-6-methoxy-7-(3-(pyrrolidin-1-yl)propoxy)quinazoline, or a pharmaceutically acceptable salt thereof, and either a mTOR-selective kinase inhibitor or a pharmaceutically acceptable salt thereof, in the manufacture of a medicament for use in the production of an antiangiogenic and/or vascular permeability reducing effect in a warm-blooded animal such as a human which is being treated with ionising radiation.

According to a further aspect of the present invention there is provided the use of 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor or a pharmaceutically acceptable salt thereof, in the manufacture of a medicament for use in the production of an anti-cancer effect in a warm-blooded animal such as a human which is being treated with ionising radiation.

According to a further aspect of the present invention there is provided the use of 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor or a pharmaceutically acceptable salt thereof, in the manufacture of a medicament for use in the production of an anti-tumour effect in a warm-blooded animal such as a human which is being treated with ionising radiation.

According to a further aspect of the present invention there is provided a therapeutic combination treatment comprising the administration of an effective amount of 4-(4-fluoro-2-methyl-1H-indol-5-yloxy)-6-methoxy-7-(3-(pyrrolidin-1-yl)propoxy)quinazoline, or a pharmaceutically acceptable salt thereof, optionally together with a pharmaceutically acceptable excipient or carrier, and the administration of an effective amount of a mTOR-selective kinase inhibitor or a pharmaceutically acceptable salt thereof, optionally together with a pharmaceutically acceptable excipient or carrier and the administration of an effective amount of ionising radiation, to a warm-blooded animal such as a human in need of such therapeutic treatment wherein the 4-(4-fluoro-2-methyl-1H-indol-5-yloxy)-6-methoxy-7-(3-(pyrrolidin-1-yl)propoxy)quinazoline, or a pharmaceutically acceptable salt thereof, the mTOR-selective kinase inhibitor and ionising radiation may be administered simultaneously, sequentially or separately and in any order.

A warm-blooded animal such as a human which is being treated with ionising radiation means a warm-blooded animal such as a human which is treated with ionising radiation before, after or at the same time as the administration of a medicament or combination treatment comprising 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor, or a pharmaceutically acceptable salt thereof. For example said ionising radiation may be given to said warm-blooded animal such as a human within the period of a week before to a week after the administration of a medicament or combination treatment comprising 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor, or a pharmaceutically acceptable salt thereof. This means that 4-(4-fluoro-2-methyl-1H-indol-5-yloxy)-6-methoxy-7-(3-(pyrrolidin-1-yl)propoxy)quinazoline, or a pharmaceutically acceptable salt thereof, the mTOR-selective kinase inhibitor, or a pharmaceutically acceptable salt thereof, and ionising radiation may be administered separately or sequentially in any order, or may be administered simultaneously. The warm-blooded animal may experience the effect of each of 4-(4-fluoro-2-methyl-1H-indol-5-yloxy)-6-methoxy-7-(3-(pyrrolidin-1-yl)propoxy)quinazoline, or a pharmaceutically acceptable salt thereof, the mTOR-selective kinase inhibitor, or a pharmaceutically acceptable salt thereof, and radiation simultaneously.

According to one aspect of the present invention the ionising radiation is administered before one of 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor, or a pharmaceutically acceptable salt thereof, or after one of 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor, or a pharmaceutically acceptable salt thereof.

According to one aspect of the present invention the ionising radiation is administered before both 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor, or a pharmaceutically acceptable salt thereof, or after both 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor, or a pharmaceutically acceptable salt thereof.

According to another aspect of the present invention the effect of a method of treatment of the present invention is expected to be at least equivalent to the addition of the effects of each of the components of said treatment used alone, that is, of each of 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor, or a pharmaceutically acceptable salt thereof, used alone or of each of 4-(4-fluoro-2-methyl-1H-indol-5-yloxy)-6-methoxy-7-(3-(pyrrolidin-1-yl)propoxy)quinazoline, or a pharmaceutically acceptable salt thereof, a mTOR-selective kinase inhibitor, or a pharmaceutically acceptable salt thereof, and ionising radiation used alone.

According to another aspect of the present invention the effect of a method of treatment of the present invention is expected to be greater than the addition of the effects of each of the components of said treatment used alone, that is, of each of 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor, or a pharmaceutically acceptable salt thereof, used alone or of each of 4-(4-fluoro-2-methyl-1H-indol-5-yloxy)-6-methoxy-7-(3-(pyrrolidin-1-yl)propoxy)quinazoline, or a pharmaceutically acceptable salt thereof, a mTOR-selective kinase inhibitor, or a pharmaceutically acceptable salt thereof, and ionising radiation used alone.

According to another aspect of the present invention the effect of a method of treatment of the present invention is expected to be a synergistic effect.

According to the present invention a combination treatment is defined as affording a synergistic effect if the effect is therapeutically superior, as measured by, for example, the extent of the response, the response rate, the time to disease progression or the survival period, to that achievable on dosing one or other of the components of the combination treatment at its conventional dose. For example, the effect of the combination treatment is synergistic if the effect is therapeutically superior to the effect achievable with 4-(4-fluoro-2-methyl-1H-indol-5-yloxy)-6-methoxy-7-(3-(pyrrolidin-1-yl)propoxy)quinazoline, or a pharmaceutically acceptable salt thereof, or a mTOR-selective kinase inhibitor, or a pharmaceutically acceptable salt thereof, or ionising radiation alone. Further, the effect of the combination treatment is synergistic if a beneficial effect is obtained in a group of patients that does not respond (or responds poorly) to 4-(4-fluoro-2-methyl-1H-indol-5-yloxy)-6-methoxy-7-(3-(pyrrolidin-1-yl)propoxy)quinazoline, or a pharmaceutically acceptable salt thereof, or a mTOR-selective kinase inhibitor, or a pharmaceutically acceptable salt thereof, or ionising radiation alone. In addition, the effect of the combination treatment is defined as affording a synergistic effect if one of the components is dosed at its conventional dose and the other component(s) is/are dosed at a reduced dose and the therapeutic effect, as measured by, for example, the extent of the response, the response rate, the time to disease progression or the survival period, is equivalent to that achievable on dosing conventional amounts of the components of the combination treatment. In particular, synergy is deemed to be present if the conventional dose of 4-(4-fluoro-2-methyl-1H-indol-5-yloxy)-6-methoxy-7-(3-(pyrrolidin-1-yl)propoxy)quinazoline, or a pharmaceutically acceptable salt thereof, or a mTOR-selective kinase inhibitor, or a pharmaceutically acceptable salt thereof, or ionising radiation may be reduced without detriment to one or more of the extent of the response, the response rate, the time to disease progression and survival data, in particular without detriment to the duration of the response, but with fewer and/or less troublesome side-effects than those that occur when conventional doses of each component are used.

In another aspect of the present invention 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor, or a pharmaceutically acceptable salt thereof, optionally with ionising radiation, are expected to inhibit the growth of those primary and recurrent solid tumours which are associated with VEGF especially those tumours which are significantly dependent on VEGF for their growth and spread.

The administration of a triple combination of 4-(4-bromo-2-fluoroanilino)-6-methoxy-7-(1-methylpiperidin-4-ylmethoxy)quinazoline, or a pharmaceutically acceptable salt thereof, a mTOR-selective kinase inhibitor, or a pharmaceutically acceptable salt thereof, and ionising radiation may produce effects, such as anti-tumour effects, greater than those achieved with any of 4-(4-bromo-2-fluoroanilino)-6-methoxy-7-(1-methylpiperidin-4-ylmethoxy)quinazoline, or a pharmaceutically acceptable salt thereof, a mTOR-selective kinase inhibitor, or a pharmaceutically acceptable salt thereof, and ionising radiation used alone, greater than those achieved with the combination of 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor, or a pharmaceutically acceptable salt thereof, greater than those achieved with the combination of 4-(4-bromo-2-fluoroanilino)-6-methoxy-7-(1-methylpiperidin-4-ylmethoxy)quinazoline, or a pharmaceutically acceptable salt thereof, and ionising radiation, greater than those achieved with the combination of a mTOR-selective kinase inhibitor, or a pharmaceutically acceptable salt thereof, and ionising radiation.

According to the present invention there is provided a method for the production of an antiangiogenic and/or vascular permeability reducing effect in a warm-blooded animal such as a human, which comprises administering to said animal an effective amount of 4-(4-bromo-2-fluoroanilino)-6-methoxy-7-(1-methylpiperidin-4-ylmethoxy)quinazoline, or a pharmaceutically acceptable salt thereof, before, after or simultaneously with an effective amount of a mTOR-selective kinase inhibitor or a pharmaceutically acceptable salt thereof, and before, after or simultaneously with an effective amount of ionising radiation.

According to a further aspect of the present invention there is provided a method for the treatment of a cancer in a warm-blooded animal such as a human, which comprises administering to said animal an effective amount of 4-(4-bromo-2-fluoroanilino)-6-methoxy-7-(1-methylpiperidin-4-ylmethoxy)quinazoline, or a pharmaceutically acceptable salt thereof, before, after or simultaneously with an effective amount of a mTOR-selective kinase inhibitor or a pharmaceutically acceptable salt thereof, and before, after or simultaneously with an effective amount of ionising radiation.

According to a further aspect of the present invention there is provided a method for the treatment of a cancer involving a solid tumour in a warm-blooded animal such as a human, which comprises administering to said animal an effective amount of 4-(4-bromo-2-fluoroanilino)-6-methoxy-7-(1-methylpiperidin-4-ylmethoxy)quinazoline, or a pharmaceutically acceptable salt thereof, before, after or simultaneously with an effective amount of a mTOR-selective kinase inhibitor or a pharmaceutically acceptable salt thereof, and before, after or simultaneously with an effective amount of ionising radiation.

According to a further aspect of the present invention there is provided a method for the production of an antiangiogenic and/or vascular permeability reducing effect in a warm-blooded animal such as a human, which comprises administering to said animal an effective amount of 4-(4-bromo-2-fluoroanilino)-6-methoxy-7-(1-methylpiperidin-4-ylmethoxy)quinazoline, or a pharmaceutically acceptable salt thereof, before, after or simultaneously with an effective amount of a mTOR-selective kinase inhibitor or a pharmaceutically acceptable salt thereof, and before, after or simultaneously with an effective amount of ionising radiation, wherein 4-(4-bromo-2-fluoroanilino)-6-methoxy-7-(1-methylpiperidin-4-ylmethoxy)quinazoline, or a pharmaceutically acceptable salt thereof, and the mTOR-selective kinase inhibitor may each optionally be administered together with a pharmaceutically acceptable excipient or carrier.

According to a further aspect of the present invention there is provided a method for the treatment of a cancer in a warm-blooded animal such as a human, which comprises administering to said animal an effective amount of 4-(4-bromo-2-fluoroanilino)-6-methoxy-7-(1-methylpiperidin-4-ylmethoxy)quinazoline, or a pharmaceutically acceptable salt thereof, before, after or simultaneously with an effective amount of a mTOR-selective kinase inhibitor or a pharmaceutically acceptable salt thereof, and before, after or simultaneously with an effective amount of ionising radiation, wherein 4-(4-bromo-2-fluoroanilino)-6-methoxy-7-(1-methylpiperidin-4-ylmethoxy)quinazoline, or a pharmaceutically acceptable salt thereof, and the mTOR-selective kinase inhibitor may each optionally be administered together with a pharmaceutically acceptable excipient or carrier.

According to a further aspect of the present invention there is provided a method for the treatment of a cancer involving a solid tumour in a warm-blooded animal such as a human, which comprises administering to said animal an effective amount of 4-(4-bromo-2-fluoroanilino)-6-methoxy-7-(1-methylpiperidin-4-ylmethoxy)quinazoline, or a pharmaceutically acceptable salt thereof, before, after or simultaneously with an effective amount of a mTOR-selective kinase inhibitor or a pharmaceutically acceptable salt thereof, and before, after or simultaneously with an effective amount of ionising radiation, wherein 4-(4-bromo-2-fluoroanilino)-6-methoxy-7-(1-methylpiperidin-4-ylmethoxy)quinazoline, or a pharmaceutically acceptable salt thereof, and the mTOR-selective kinase inhibitor may each optionally be administered together with a pharmaceutically acceptable excipient or carrier.

According to a further aspect of the present invention there is provided the use of 4-(4-bromo-2-fluoroanilino)-6-methoxy-7-(1-methylpiperidin-4-ylmethoxy)quinazoline, or a pharmaceutically acceptable salt thereof, and either a mTOR-selective kinase inhibitor or a pharmaceutically acceptable salt thereof, in the manufacture of a medicament for use in the production of an antiangiogenic and/or vascular permeability reducing effect in a warm-blooded animal such as a human which is being treated with ionising radiation.

According to a further aspect of the present invention there is provided the use of 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor or a pharmaceutically acceptable salt thereof, in the manufacture of a medicament for use in the production of an anti-cancer effect in a warm-blooded animal such as a human which is being treated with ionising radiation.

According to a further aspect of the present invention there is provided the use of 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor or a pharmaceutically acceptable salt thereof, in the manufacture of a medicament for use in the production of an anti-tumour effect in a warm-blooded animal such as a human which is being treated with ionising radiation.

According to a further aspect of the present invention there is provided a therapeutic combination treatment comprising the administration of an effective amount of 4-(4-bromo-2-fluoroanilino)-6-methoxy-7-(1-methylpiperidin-4-ylmethoxy)quinazoline, or a pharmaceutically acceptable salt thereof, optionally together with a pharmaceutically acceptable excipient or carrier, and the administration of an effective amount of a mTOR-selective kinase inhibitor or a pharmaceutically acceptable salt thereof, optionally together with a pharmaceutically acceptable excipient or carrier and the administration of an effective amount of ionising radiation, to a warm-blooded animal such as a human in need of such therapeutic treatment wherein the 4-(4-bromo-2-fluoroanilino)-6-methoxy-7-(1-methylpiperidin-4-ylmethoxy)quinazoline, or a pharmaceutically acceptable salt thereof, the mTOR-selective kinase inhibitor and ionising radiation may be administered simultaneously, sequentially or separately and in any order.

A warm-blooded animal such as a human which is being treated with ionising radiation means a warm-blooded animal such as a human which is treated with ionising radiation before, after or at the same time as the administration of a medicament or combination treatment comprising 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor, or a pharmaceutically acceptable salt thereof. For example said ionising radiation may be given to said warm-blooded animal such as a human within the period of a week before to a week after the administration of a medicament or combination treatment comprising 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor, or a pharmaceutically acceptable salt thereof. This means that 4-(4-bromo-2-fluoroanilino)-6-methoxy-7-(1-methylpiperidin-4-ylmethoxy)quinazoline, or a pharmaceutically acceptable salt thereof, the mTOR-selective kinase inhibitor, or a pharmaceutically acceptable salt thereof, and ionising radiation may be administered separately or sequentially in any order, or may be administered simultaneously. The warm-blooded animal may experience the effect of each of 4-(4-bromo-2-fluoroanilino)-6-methoxy-7-(1-methylpiperidin-4-ylmethoxy)quinazoline, or a pharmaceutically acceptable salt thereof, the mTOR-selective kinase inhibitor, or a pharmaceutically acceptable salt thereof, and radiation simultaneously.

According to one aspect of the present invention the ionising radiation is administered before one of 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor, or a pharmaceutically acceptable salt thereof, or after one of 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor, or a pharmaceutically acceptable salt thereof.

According to one aspect of the present invention the ionising radiation is administered before both 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor, or a pharmaceutically acceptable salt thereof, or after both 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor, or a pharmaceutically acceptable salt thereof.

According to another aspect of the present invention the effect of a method of treatment of the present invention is expected to be at least equivalent to the addition of the effects of each of the components of said treatment used alone, that is, of each of 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor, or a pharmaceutically acceptable salt thereof, used alone or of each of 4-(4-bromo-2-fluoroanilino)-6-methoxy-7-(1-methylpiperidin-4-ylmethoxy)quinazoline, or a pharmaceutically acceptable salt thereof, a mTOR-selective kinase inhibitor, or a pharmaceutically acceptable salt thereof, and ionising radiation used alone.

According to another aspect of the present invention the effect of a method of treatment of the present invention is expected to be greater than the addition of the effects of each of the components of said treatment used alone, that is, of each of 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor, or a pharmaceutically acceptable salt thereof, used alone or of each of 4-(4-bromo-2-fluoroanilino)-6-methoxy-7-(1-methylpiperidin-4-ylmethoxy)quinazoline, or a pharmaceutically acceptable salt thereof, a mTOR-selective kinase inhibitor, or a pharmaceutically acceptable salt thereof, and ionising radiation used alone.

According to another aspect of the present invention the effect of a method of treatment of the present invention is expected to be a synergistic effect.

According to the present invention a combination treatment is defined as affording a synergistic effect if the effect is therapeutically superior, as measured by, for example, the extent of the response, the response rate, the time to disease progression or the survival period, to that achievable on dosing one or other of the components of the combination treatment at its conventional dose. For example, the effect of the combination treatment is synergistic if the effect is therapeutically superior to the effect achievable with 4-(4-bromo-2-fluoroanilino)-6-methoxy-7-(1-methylpiperidin-4-ylmethoxy)quinazoline, or a pharmaceutically acceptable salt thereof, or a mTOR-selective kinase inhibitor, or a pharmaceutically acceptable salt thereof, or ionising radiation alone. Further, the effect of the combination treatment is synergistic if a beneficial effect is obtained in a group of patients that does not respond (or responds poorly) to 4-(4-bromo-2-fluoroanilino)-6-methoxy-7-(1-methylpiperidin-4-ylmethoxy)quinazoline, or a pharmaceutically acceptable salt thereof, or a mTOR-selective kinase inhibitor, or a pharmaceutically acceptable salt thereof, or ionising radiation alone. In addition, the effect of the combination treatment is defined as affording a synergistic effect if one of the components is dosed at its conventional dose and the other component(s) is/are dosed at a reduced dose and the therapeutic effect, as measured by, for example, the extent of the response, the response rate, the time to disease progression or the survival period, is equivalent to that achievable on dosing conventional amounts of the components of the combination treatment. In particular, synergy is deemed to be present if the conventional dose of 4-(4-bromo-2-fluoroanilino)-6-methoxy-7-(1-methylpiperidin-4-ylmethoxy)quinazoline, or a pharmaceutically acceptable salt thereof, or a mTOR-selective kinase inhibitor, or a pharmaceutically acceptable salt thereof, or ionising radiation may be reduced without detriment to one or more of the extent of the response, the response rate, the time to disease progression and survival data, in particular without detriment to the duration of the response, but with fewer and/or less troublesome side-effects than those that occur when conventional doses of each component are used.

In another aspect of the present invention 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor, or a pharmaceutically acceptable salt thereof, optionally with ionising radiation, are expected to inhibit the growth of those primary and recurrent solid tumours which are associated with VEGF especially those tumours which are significantly dependent on VEGF for their growth and spread.

Radiotherapy may be administered according to the known practices in clinical radiotherapy. The dosages of ionising radiation will be those known for use in clinical radiotherapy. The radiation therapy used will include for example the use of γ-rays, X-rays, and/or the directed delivery of radiation from radioisotopes. Other forms of DNA damaging factors are also included in the present invention such as microwaves and UV-irradiation. For example X-rays may be dosed in daily doses of 1.8-2.0 Gy, 5 days a week for 5-6 weeks. Normally a total fractionated dose will lie in the range 45-60 Gy. Single larger doses, for example 5-10 Gy may be administered as part of a course of radiotherapy. Single doses may be administered intraoperatively. Hyperfractionated radiotherapy may be used whereby small doses of X-rays are administered regularly over a period of time, for example 0.1 Gy per hour over a number of days. Dosage ranges for radioisotopes vary widely, and depend on the half-life of the isotope, the strength and type of radiation emitted, and on the uptake by cells.

Other chemotherapeutic agents for optional use with a combination treatment of the present invention include may include one or more of the following categories of anti tumor agents:

(i) Other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolomide and nitrosoureas); antimetabolites (mercaptopurine, fludarabine, gemcitabine fluoropyrimidines such as 5-fluorouracil, capecitabine and tegafur, raltitrexed, methotrexate, cytosine arabinoside; antitumour antibiotics (anthracyclines such as adriamycin, bleomycin, daunomycin, mitoxantrone, epirubicin, idarubicin, mitomycin-C, antibiotics from streptomyces such as hydroxyurea, dactinomycin, bleomycin and mithramycin); antimitotic agents (for example vinca alkaloids like vincristine, vinblastine, vindesine and vinorelbine and taxanes like paclitaxel and docetaxal and polokinase inhibitors); and topoisomerase inhibitors (for example epipodophyllotoxins like etoposide and teniposide, amsacrine, topotecan and irinotecan);
(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of
5α-reductase such as finasteride;
(iii) anti-invasion agents (for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341) and N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661), and metalloproteinase inhibitors like marimastat, inhibitors of urokinase plasminogen activator receptor function or antibodies to Heparanase);
(iv) inhibitors of growth factor function: for example such inhibitors include growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stern et al. Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib, inhibitors of the hepatocyte growth factor family, inhibitors of the platelet-derived growth factor family such as imatinib, inhibitors of serine/threonine kinases (for example Ras signalling inhibitors such as farnesyl transferase inhibitors, for example sorafenib (BAY 43-9006)), inhibitors of cell signalling through AKT kinases, inhibitors of the hepatocyte growth factor family, c-kit inhibitors, abl kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;
(v) antiangiogenic agents such as those which inhibit the effects of vascular endothelial growth factor, [for example the anti-vascular endothelial cell growth factor antibody bevacizumab (Avastin™) and VEGF receptor tyrosine kinase inhibitors such as 4-(4-bromo-2-fluoroanilino)-6-methoxy-7-(1-methylpiperidin-4-ylmethoxy)quinazoline (ZD6474; Example 2 within WO 01/32651), vatalanib (PTK787; WO 98/35985) and SU11248 (sunitinib; WO 01/60814), compounds such as those disclosed in International Patent Applications WO 97/22596, WO 97/30035, WO 97/32856 and WO 98/13354 and compounds that work by other mechanisms (for example linomide, inhibitors of integrin avβ3 function and angiostatin)];
(vi) vascular damaging agents such as Combretastatin A4 and compounds disclosed in International Patent Applications WO 99/02166, WO 00/40529, WO 00/41669, WO 01/92224, WO 02/04434 and WO 02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

Particular examples of chemotherapeutic agents for use with a combination treatment of the present invention are pemetrexed, raltitrexed, etoposide, vinorelbine, paclitaxel, docetaxel, cisplatin, oxaliplatin, carboplatin, gemcitabine, irinotecan (CPT-11), topotecan, 5-fluorouracil (5-FU, (including capecitabine)), doxorubicin, anthracyclines, bleomycin, cyclophosphamide, temozolomide, nitrosureas and hydroxyurea. Such combinations are expected to be particularly useful for the treatment patients with lung cancer, prostate cancer, melanoma, ovarian cancer, breast cancer, endometrial cancer, kidney cancer, sarcomas, head and neck cancers, gastric cancer, tumours of the central nervous system and their metastases, and also for the treatment of patients with acute myeloid leukaemia.

For the avoidance of doubt, according to the present invention the mTOR-selective kinase inhibitor is selected from any one of
5-[2,4-bis[3-methylmorpholin-4-yl]pyrido[5,6-e]pyrimidin-7-yl]-2-methoxy-N-methylbenzamide;
4-[2,4-bis[3-methylmorpholin-4-yl]pyrido[5,6-e]pyrimidin-7-yl]aniline;
6-[2,4-bis[3-methylmorpholin-4-yl]pyrido[5,6-e]pyrimidin-7-yl]-1H-indazol-3-amine;
8-[2,4-bis[3-methylmorpholin-4-yl]pyrido[5,6-e]pyrimidin-7-yl]-1,2,3,4-tetrahydro-1,4-benzodiazepin-5-one;
5-[2,4-bis[3-methylmorpholin-4-yl]pyrido[5,6-e]pyrimidin-7-yl]-2-methoxy-N-methylbenzamide;
5-[2,4-bis[3-methylmorpholin-4-yl]pyrido[5,6-e]pyrimidin-7-yl]pyridin-2-amine;
N-[3-[2,4-bis[3-methylmorpholin-4-yl]pyrido[5,6-e]pyrimidin-7-yl]phenyl]methanesulfonamide;
3-[2,4-bis[3-methylmorpholin-4-yl]pyrido[5,6-e]pyrimidin-7-yl]aniline;
5-[2,4-bis[3-methylmorpholin-4-yl]pyrido[6,5-d]pyrimidin-7-yl]-2-ethoxybenzamide;
5-[2,4-bis[3-methylmorpholin-4-yl]pyrido[5,6-e]pyrimidin-7-yl]-2-(difluoromethoxy)-N-methylbenzamide;
5-[2,4-bis[3-methylmorpholin-4-yl]pyrido[5,6-e]pyrimidin-7-yl]-1H-indazol-3-amine;
[5-[2,4-bis[3-methylmorpholin-4-yl]pyrido[5,6-e]pyrimidin-7-yl]-2-methoxyphenyl]methanol;
N-[[4-[2,4-bis[3-methylmorpholin-4-yl]pyrido[5,6-e]pyrimidin-7-yl]phenyl]methyl]methanesulfonamide;
5-[2,4-bis[3-methylmorpholin-4-yl]pyrido[5,6-e]pyrimidin-7-yl]-1,3-dihydroindol-2-one;
6-[2,4-bis[3-methylmorpholin-4-yl]pyrido[5,6-e]pyrimidin-7-yl]-1,3-dihydroindol-2-one;
3-[2,4-bis[3-methylmorpholin-4-yl]pyrido[5,6-e]pyrimidin-7-yl]-N-methylbenzamide;
5-[2,4-bis[3-methylmorpholin-4-yl]pyrido[5,6-e]pyrimidin-7-yl]-2-(difluoromethoxy)benzamide;
6-[2,4-bis[3-methylmorpholin-4-yl]pyrido[5,6-e]pyrimidin-7-yl]-2,3-dihydroisoindol-1-one;
[5-[2-(2,6-dimethylmorpholin-4-yl)-4-morpholin-4-ylpyrido[6,5-d]pyrimidin-7-yl]-2-methoxyphenyl]methanol; and
[2-methoxy-5-[2-(3-methylmorpholin-4-yl)-4-morpholin-4-ylpyrido[6,5-d]pyrimidin-7-yl]phenyl]methanol, or a pharmaceutically acceptable salt thereof.

In one embodiment the mTOR-selective kinase inhibitor is selected from any one of
5-[2,4-bis[(3S)-3-methylmorpholin-4-yl]pyrido[5,6-e]pyrimidin-7-yl]-2-methoxy-N-methylbenzamide;
4-[2,4-bis[(3S)-3-methylmorpholin-4-yl]pyrido[5,6-e]pyrimidin-7-yl]aniline;
6-[2,4-bis[(3S)-3-methylmorpholin-4-yl]pyrido[5,6-e]pyrimidin-7-yl]-1H-indazol-3-amine;
8-[2,4-bis[(3S)-3-methylmorpholin-4-yl]pyrido[5,6-e]pyrimidin-7-yl]-1,2,3,4-tetrahydro-1,4-benzodiazepin-5-one;
5-[2,4-bis[(3S)-3-methylmorpholin-4-yl]pyrido[5,6-e]pyrimidin-7-yl]-2-methoxy-N-methylbenzamide;
5-[2,4-bis[(3S)-3-methylmorpholin-4-yl]pyrido[5,6-e]pyrimidin-7-yl]pyridin-2-amine;
N-[3-[2,4-bis[(3S)-3-methylmorpholin-4-yl]pyrido[5,6-e]pyrimidin-7-yl]phenyl]methanesulfonamide;
3-[2,4-bis[(3S)-3-methylmorpholin-4-yl]pyrido[5,6-e]pyrimidin-7-yl]aniline;
5-[2,4-bis[(3S)-3-methylmorpholin-4-yl]pyrido[6,5-d]pyrimidin-7-yl]-2-ethoxybenzamide;
5-[2,4-bis[(3S)-3-methylmorpholin-4-yl]pyrido[5,6-e]pyrimidin-7-yl]-2-(difluoromethoxy)-N-methylbenzamide;
5-[2,4-bis[(3S)-3-methylmorpholin-4-yl]pyrido[5,6-e]pyrimidin-7-yl]-1H-indazol-3-amine;
[5-[2,4-bis[(3S)-3-methylmorpholin-4-yl]pyrido[5,6-e]pyrimidin-7-yl]-2-methoxyphenyl]methanol;
N-[[4-[2,4-bis[(3S)-3-methylmorpholin-4-yl]pyrido[5,6-e]pyrimidin-7-yl]phenyl]methyl]methanesulfonamide;
5-[2,4-bis[(3S)-3-methylmorpholin-4-yl]pyrido[5,6-e]pyrimidin-7-yl]-1,3-dihydroindol-2-one;
6-[2,4-bis[(3S)-3-methylmorpholin-4-yl]pyrido[5,6-e]pyrimidin-7-yl]-1,3-dihydroindol-2-one;
3-[2,4-bis[(3S)-3-methylmorpholin-4-yl]pyrido[5,6-e]pyrimidin-7-yl]-N-methylbenzamide;
5-[2,4-bis[(3S)-3-methylmorpholin-4-yl]pyrido[5,6-e]pyrimidin-7-yl]-2-(difluoromethoxy)benzamide;
6-[2,4-bis[(3S)-3-methylmorpholin-4-yl]pyrido[5,6-e]pyrimidin-7-yl]-2,3-dihydroisoindol-1-one;
[5-[2-(2,6-dimethylmorpholin-4-yl)-4-morpholin-4-ylpyrido[6,5-d]pyrimidin-7-yl]-2-methoxyphenyl]methanol; and
[2-methoxy-5-[2-(3-methylmorpholin-4-yl)-4-morpholin-4-ylpyrido[6,5-d]pyrimidin-7-yl]phenyl]methanol, or a pharmaceutically acceptable salt thereof.

In one embodiment the mTOR-selective kinase inhibitor is [5-[2,4-bis[(3S)-3-methylmorpholin-4-yl]pyrido[5,6-e]pyrimidin-7-yl]-2-methoxyphenyl]methanol, or a pharmaceutically acceptable salt thereof.

Compounds have been named with the aid of computer software (Lexichem 1.6 from Openeye).

In one embodiment the mTOR-selective kinase inhibitor is selective for mTOR over PI3K. In one embodiment the mTOR-selective kinase inhibitor is greater than 2 fold selective for mTOR over PI3K. In one embodiment the mTOR-selective kinase inhibitor is greater than 10 fold selective for mTOR over PI3K. In one embodiment the mTOR-selective kinase inhibitor is greater than 100 fold selective for mTOR over PI3K. In one embodiment the mTOR-selective kinase inhibitor inhibits TORC2. In one embodiment the mTOR-selective kinase inhibitor inhibits TORC1 and TORC2.

### General Synthesis

4-(4-fluoro-2-methyl-1H-indol-5-yloxy)-6-methoxy-7-(3-(pyrrolidin-1-yl)propoxy)quinazoline may be synthesised according to the processes described in WO 00/47212, in particular those described in Example 240 of WO 00/47212.

mTOR-selective kinase inhibitor compounds can be represented by Formula 1:

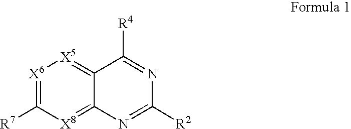

wherein R⁴ represents NR^{N3}R^{N4},
and wherein:
one or two of X⁵, X⁶ and X⁸ is N, and the others are CH;
R⁷ is selected from halo, OR^{O1}, SR^{S1}, NR^{N1}R^{N2}, NR^{N7a}C(═O)R^{C1}, NR^{N7b}SO₂R^{S2a}, an optionally substituted C₅₋₂₀ heteroaryl group, or an optionally substituted C₅₋₂₀ aryl group,
where R^{O1} and R^{S1} are selected from H, an optionally substituted C₅₋₂₀ aryl group, an optionally substituted C₅₋₂₀ heteroaryl group, or an optionally substituted C₁₋₇ alkyl group;
R^{N1} and R^{N2} are independently selected from H, an optionally substituted C₁₋₇ alkyl group, an optionally substituted C₅₋₂₀ heteroaryl group, an optionally substituted C₅₋₂₀ aryl group or R^{N1} and R^{N2} together with the nitrogen to which they are bound form a heterocyclic ring containing between 3 and 8 ring atoms;
R^{C1} is selected from H, an optionally substituted C₅₋₂₀ aryl group, an optionally substituted C₅₋₂₀ heteroaryl group, an optionally substituted C₁₋₇ alkyl group or NR^{N8}R^{N9}, where R^{N8} and R^{N9} are independently selected from H, an optionally substituted C₁₋₇ alkyl group, an optionally substituted C₅₋₂₀ heteroaryl group, an optionally substituted C₅₋₂₀ aryl group or R^{N8} and R^{N9} together with the nitrogen to which they are bound form a heterocyclic ring containing between 3 and 8 ring atoms;
R^{S2a} is selected from H, an optionally substituted C₅₋₂₀ aryl group, an optionally substituted C₅₋₂₀ heteroaryl group, or an optionally substituted C₁₋₇ alkyl group;
R^{N7a} and R^{N7b} are selected from H and a C₁₋₄ alkyl group;
R^{N3} and R^{N4}, together with the nitrogen to which they are bound, form a heterocyclic ring containing between 3 and 8 ring atoms;
R² is selected from H, halo, OR^{O2}, SR^{S2b}, NR^{N5}R^{N6}, an optionally substituted C₅₋₂₀ heteroaryl group, and an optionally substituted C₅₋₂₀ aryl group,
wherein R^{O2} and R^{S2b} are selected from H, an optionally substituted C₅₋₂₀ aryl group, an optionally substituted C₅₋₂₀ heteroaryl group, or an optionally substituted C₁₋₇ alkyl group;
R^{N5} and R^{N6} are independently selected from H, an optionally substituted C₁₋₇ alkyl group, an optionally substituted C₅₋₂₀ heteroaryl group, and an optionally substituted C₅₋₂₀ aryl group, or R^{N5} and R^{N6} together with the nitrogen to which they are bound form a heterocyclic ring containing between 3 and 8 ring atoms,
or a pharmaceutically acceptable salt thereof,
with the proviso that when R² is unsubstituted morpholino, R^{N3} and R^{N4} together with the nitrogen atom to which they are attached form an unsubstituted morpholino and R⁷ is unsubstituted phenyl, and X⁵ is CH, then X⁶ is not N and X⁸ is not CH, or X⁶ is not CH and X⁸ is not N, and
when R² is unsubstituted piperidinyl, R^{N3} and R^{N4} together with the nitrogen atom to which they are attached form an unsubstituted piperidinyl and R⁷ is unsubstituted phenyl, and X⁵ is CH, then X⁶ is not CH and X⁸ is not N.

Compounds of Formula 1 can be synthesised from compounds of Formula 2:

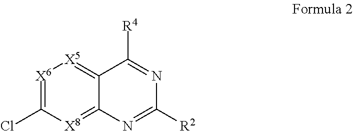

When R⁷ is NR^{N1}R^{N2}, this is by reaction with R⁷H. When R⁷ is an optionally substituted C₃₋₂₀ heterocyclyl group or C₅₋₂₀ aryl group, this is by reaction with R⁷B(OAlk)₂, where each Alk is independently C₁₋₇ alkyl or together with the oxygen to which they are attached form a C₅₋₇ heterocyclyl group. When R⁷ is an amide, urea or sulfonamide group, this is by reaction with ammonia followed by reaction of the resulting primary amide with the appropriate acid chloride, isocyanate or sulfonyl chloride. When R⁷ is OR^{O1} or SR^{S1}, this is by reaction with potassium carbonate in the appropriate alcohol or thiol solvent.

Therefore, according to a further aspect of the present invention there is provided a process for the preparation of a compound of formula I, from a compound of Formula 2:

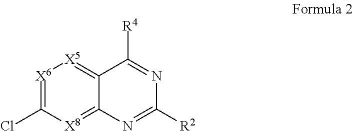

wherein:
R⁴ is NR^{N3}R^{N4} where R^{N3} and R^{N4}, together with the nitrogen to which they are bound, form a heterocyclic ring containing between 3 and 8 ring atoms;
R² is selected from H, halo, OR^{O2}, SR^{S2b}, NR^{N5}R^{N6}, an optionally substituted C₅₋₂₀ heteroaryl group, and an optionally substituted C₅₋₂₀ aryl group,
wherein R^{O2} and R^{S2b} are selected from H, an optionally substituted C₅₋₂₀ aryl group, an optionally substituted C₅₋₂₀ heteroaryl group, or an optionally substituted C₁₋₇ alkyl group, and R^{N5} and R^{N6} are independently selected from H, an optionally substituted C₁₋₇ alkyl group, an optionally substituted C₅₋₂₀ heteroaryl group, and an optionally substituted C₅₋₂₀ aryl group, or R^{N5} and R^{N6} together with the nitrogen to which they are bound form a heterocyclic ring containing between 3 and 8 ring atoms,
comprising
(a) when R⁷ is NR^{N1}R^{N2}, reaction of the compound of formula 2 with R⁷H; or
(b) when R⁷ is an optionally substituted C₃₋₂₀ heterocyclyl group or C₅₋₂₀ aryl group, reaction of the compound of formula 2 with R⁷B(OAlk)₂, where each Alk is independently C₁₋₇ alkyl or together with the oxygen to which they are attached form a C₅₋₇ heterocyclyl group, or
(c) when R⁷ is an amide, urea or sulfonamide group, reaction of a compound of formula 2 with ammonia followed by reaction of the resulting primary amine with the appropriate acid chloride, isocyanate or sulfonyl chloride, or
(d) when R⁷ is OR^{O1} or SR^{S1}, by reaction of the compound of formula 1 in the presence of base in the appropriate alcohol or thiol solvent.

Compounds of formula I(A) can be synthesised by reaction a compound of Formula 1a:

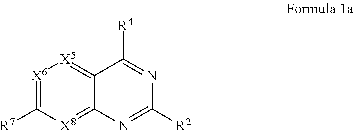

wherein R⁴ represents NR^{N3}R^{N4}, and

### R⁷ is

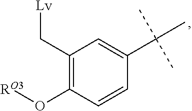

wherein Lv is a leaving group, such as a halogen, for example chlorine, or an OSO₂R group, where R is alkyl or aryl, such as methyl,
by reaction with R^{N10}NH₂.

Compounds of Formula 1a can be synthesised by reaction of a compound of Formula 1b

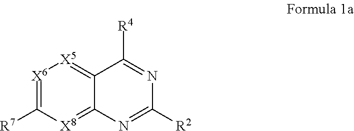

wherein R⁴ represents NR^{N3}R^{N4}, and

### R⁷ is

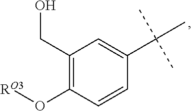

with an alkyl or aryl sulphonyl chloride in the presence of a base.

Compounds of Formula 1b can be synthesised by reacting a compound of Formula 2:

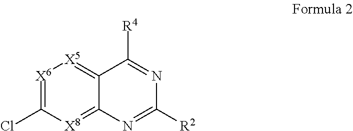

with R⁷B(OAlk)₂, where each Alk is independently C₁₋₇ alkyl or together with the oxygen to which they are attached form a C₅₋₇ heterocyclyl group.

Compounds of Formula 2 can be synthesised from compounds of Formula 3:

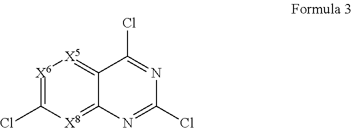

by reaction with HR⁴ (HNR^{N3}R^{N4}) followed by reaction with HR².

Compounds of Formula 3 can be synthesised from compounds of Formula 4:

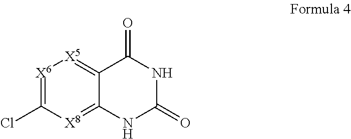

by treatment with POCl₃ and N,N-diisopropylamine, for example.

Compounds of Formula 4 can be synthesised from compounds of Formula 5:

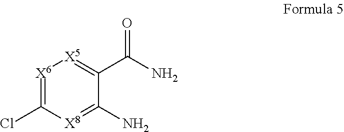

by treatment with oxalyl chloride, for example.

Compounds of Formula 5 can be synthesised from compounds of Formula 6, for example by reaction with liquid ammonia followed by reaction with thionyl chloride and ammonia gas:

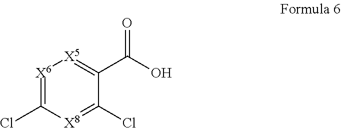

Alternatively, Compounds of Formula 1 can be synthesised from compounds of Formula 2A:

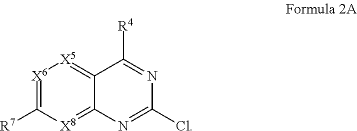

When R² is NR^{N5}R^{N6}, this is by reaction with R²H. When R² is an optionally substituted C₃₋₂₀ heterocyclyl group or C₅₋₂₀ aryl group, this is by reaction with R²B(OAlk)₂, where each Alk is independently C₁₋₇ alkyl or together with the oxygen to which they are attached form a C₅₋₇ heterocyclyl group. When R² is OR^{O2} or SR^{S2b}, this is by reaction with potassium carbonate in the appropriate alcohol or thiol solvent.

Therefore, according to a further aspect of the present invention there is provided a process for the preparation of a compound of formula 1 from a compound of formula 2A:

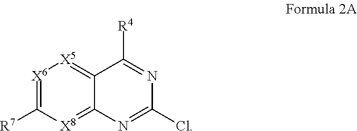

wherein
R⁴ is NR^{N3}R^{N4} where R^{N3} and R^{N4}, together with the nitrogen to which they are bound, form a heterocyclic ring containing between 3 and 8 ring atoms; and
R⁷ is selected from halo, OR^{O1}, SR^{S1}, NR^{N1}R^{N2}, NR^{N7a}C(═O)R^{C1}, NR^{N7b}SO₂R^{S2a}, an optionally substituted C₅₋₂₀ heteroaryl group, or an optionally substituted C₅₋₂₀ aryl group, where R^{O1} and R^{S1} are selected from H, an optionally substituted C₅₋₂₀ aryl group, an optionally substituted C₅₋₂₀ heteroaryl group, or an optionally substituted C₁₋₇ alkyl group;
R^{N1} and R^{N2} are independently selected from H, an optionally substituted C₁₋₇ alkyl group, an optionally substituted C₅₋₂₀ heteroaryl group, an optionally substituted C₅₋₂₀ aryl group or R^{N1} and R^{N2} together with the nitrogen to which they are bound form a heterocyclic ring containing between 3 and 8 ring atoms;
R^{C1} is selected from H, an optionally substituted C₅₋₂₀ aryl group, an optionally substituted C₅₋₂₀ heteroaryl group, an optionally substituted C₁₋₇ alkyl group or NR^{N8}R^{N9}, where R^{N8} and R^{N9} are independently selected from H, an optionally substituted C₁₋₇ alkyl group, an optionally substituted C₅₋₂₀ heteroaryl group, an optionally substituted C₅₋₂₀ aryl group or R^{N8} and R^{N9} together with the nitrogen to which they are bound form a heterocyclic ring containing between 3 and 8 ring atoms;
R^{S2a} is selected from H, an optionally substituted C₅₋₂₀ aryl group, an optionally substituted C₅₋₂₀ heteroaryl group, or an optionally substituted C₁₋₇ alkyl group; and
R^{N7a} and R^{N7b} are selected from H and a C₁₋₄ alkyl group;
comprising
(a) when R² is NR^{N5}R^{N6}, reacting a compound of formula 2A with R²H, or
(b) when R² is an optionally substituted C₃₋₂₀ heterocyclyl group or C₅₋₂₀ aryl group, by reacting a compound of formula 2A with R²B(OAlk)₂, where each Alk is independently C₁₋₇ alkyl or together with the oxygen to which they are attached form a C₅₋₇ heterocyclyl group, or
(c) when R² is OR^{O2} or SR^{S2b}, by reacting a compound of formula 2A in the presence of a base in the appropriate alcohol or thiol solvent.

Compounds of Formula 2A can be synthesised from compounds of Formula 3:

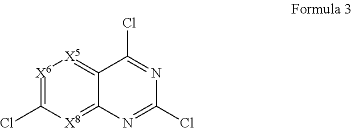

by reaction with HR⁴ (HNR^{N3}R^{N4}) followed by reaction with HR⁷ or HR⁷ equivalent. For example, when R⁷ is an optionally substituted C₃₋₂₀ heterocyclyl group or C₅₋₂₀ aryl group, this is by reaction with R⁷B(OAlk)₂, where each Alk is independently C₁₋₇ alkyl or together with the oxygen to which they are attached form a C₅₋₇ heterocyclyl group.

Compounds of formula I(B) can be represented by Formula 1.1:

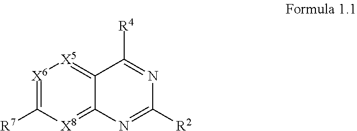

wherein R⁴ represents

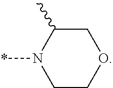

Compounds of Formula 1.1 can be synthesised from compounds of Formula 2.1:

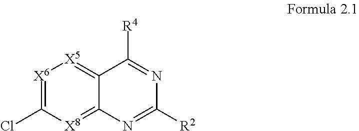

wherein R⁴ represents

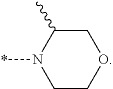

When R⁷ is NR^{N1}R^{N2}, this is by reaction with R⁷H. When R⁷ is an amide, urea or sulfonamide group, this is by reaction with ammonia followed by reaction of the resulting primary amide with the appropriate acid chloride, isocyanate or sulfonyl chloride. When R⁷ is OR^{O1} or SR^{S1}, this is by reaction with potassium carbonate in the appropriate alcohol or thiol solvent. When R⁷ is an optionally substituted C₃₋₂₀ heterocyclyl group or C₅₋₂₀ aryl group, this is by reaction with R⁷B(OAlk)₂, where each Alk is independently C₁₋₇ alkyl or together with the oxygen to which they are attached form a C₅₋₇ heterocyclyl group.

Compounds of Formula 2.1 can be synthesised from compounds of Formula 3:

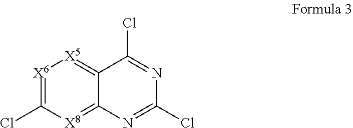

by reaction with HR⁴

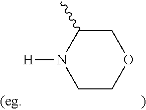

followed by reaction with HR².

Alternatively compounds of Formula 1 and Formula 1.1 can be synthesised from compounds of Formula 7:

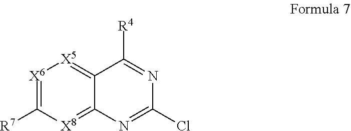

by reaction with HR².

Compounds of Formula 7 can be synthesised from compounds of Formula 8:

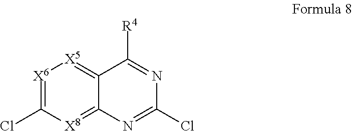

When R⁷ is NR^{N1}R^{N2}, this is by reaction with R⁷H. When R⁷ is an amide, urea or sulfonamide group, this is by reaction with ammonia followed by reaction of the resulting primary amide with the appropriate acid chloride, isocyanate or sulfonyl chloride. When R⁷ is OR^{O1} or SR^{S1}, this is by reaction with potassium carbonate in the appropriate alcohol or thiol solvent. When R⁷ is an optionally substituted C₃₋₂₀ heterocyclyl group or C₅₋₂₀ aryl group, this is by reaction with R⁷B(OAlk)₂, where each Alk is independently C₁₋₇ alkyl or together with the oxygen to which they are attached form a C₅₋₇ heterocyclyl group.

Compounds of Formula 8 can be synthesised from compounds of Formula 3:

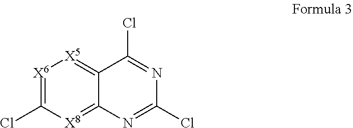

by reaction with HR⁴

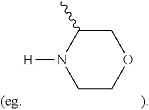

When R⁷ is

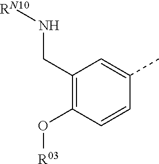

the Compound of Formula II can be prepared by reaction a compound of Formula 1.2:

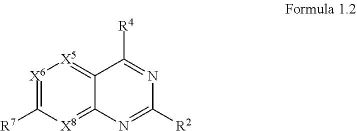

wherein R⁴ represents

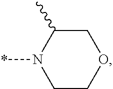

and

### R⁷ is

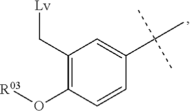

wherein Lv is a leaving group, such as a halogen, for example chlorine, or a OSO₂ group, where R is alkyl or aryl, such as methyl, by reaction with R^{N10}NH₂.

Compounds of Formula 1.2 can be synthesised by reaction of a compound of Formula 1.3

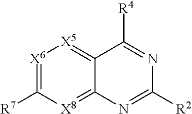

wherein R⁴ represents

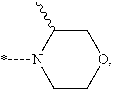

and

### R⁷ is

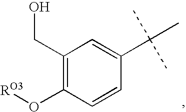

with an alkyl or aryl sulphonyl chloride in the presence of a base.

For Example:

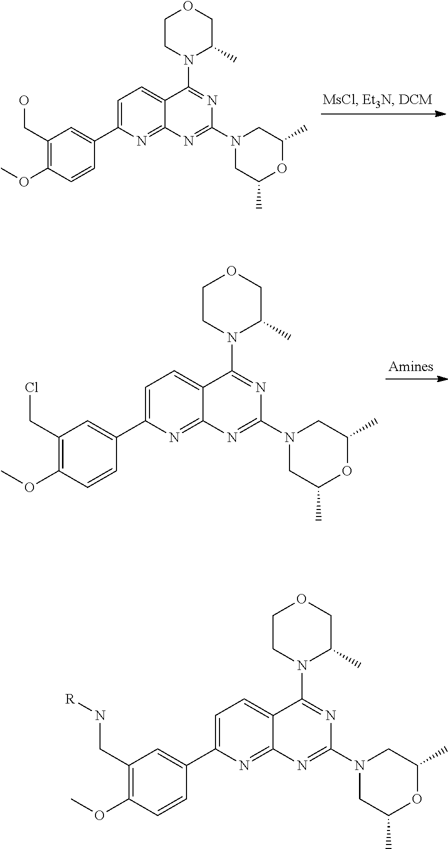

Compounds of Formula 1.3 can be prepared by reaction with R⁷B(OAlk)₂, where each Alk is independently C₁₋₇ alkyl or together with the oxygen to which they are attached form a C₅₋₇ heterocyclyl group.

It may be convenient or desirable to prepare, purify, and/or handle a corresponding salt of the inhibitor, for example, a pharmaceutically-acceptable salt.

Salts of 4-(4-fluoro-2-methyl-1H-indol-5-yloxy)-6-methoxy-7-(3-(pyrrolidin-1-yl)propoxy)quinazoline or a mTOR-selective kinase inhibitor for use in pharmaceutical compositions will be pharmaceutically acceptable salts, but other salts may be useful in the production of 4-(4-fluoro-2-methyl-1H-indol-5-yloxy)-6-methoxy-7-(3-(pyrrolidin-1-yl)propoxy)quinazoline or a mTOR-selective kinase inhibitor and their pharmaceutically acceptable salts. Pharmaceutically acceptable salts may, for example, include acid addition salts. Such acid-addition salts include but are not limited to, furmarate, methanesulfonate, hydrochloride, trifluoroacetate, hydrobromide, citrate and maleate salts and salts formed with phosphoric and sulfuric acid. In addition pharmaceutically acceptable salts may be formed with an inorganic or organic base which affords a pharmaceutically acceptable cation. Such salts formed with inorganic and organic bases include alkali or alkaline earth metal salts and organic amine salts. Alkali or alkaline earth metal salts include but are not limited to, an alkali metal salt for example sodium or potassium, an alkaline earth metal salt for example calcium or magnesium. Organic amine salts include but are not limited to triethylamine, ethanolamine, diethanolamine, triethanolamine, morpholine, N-methylpiperidine, N-ethylpiperidine, dibenzylamine or amino acids such as lysine.

A preferred salt is 4-(4-fluoro-2-methyl-1H-indol-5-yloxy)-6-methoxy-7-(3-(pyrrolidin-1-yl)propoxy)quinazoline maleate which is described in International Patent Application Publication No. WO 05/061488.

4-(4-fluoro-2-methyl-1H-indol-5-yloxy)-6-methoxy-7-(3-(pyrrolidin-1-yl)propoxy)quinazoline maleate salt may be synthesised according to the processes described in WO 05/061488.

A formulation of 4-(4-fluoro-2-methyl-1H-indol-5-yloxy)-6-methoxy-7-(3-(pyrrolidin-1-yl)propoxy)quinazoline, or a pharmaceutically acceptable salt thereof, or a mTOR-selective kinase inhibitor, or a pharmaceutically acceptable salt thereof, (each of which is an “active compound”), comprises 4-(4-fluoro-2-methyl-1H-indol-5-yloxy)-6-methoxy-7-(3-(pyrrolidin-1-yl)propoxy)quinazoline, or a pharmaceutically acceptable salt thereof, or a mTOR-selective kinase inhibitor, or a pharmaceutically acceptable salt thereof, as defined herein, together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents. A combined preparation of 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor, or a pharmaceutically acceptable salt thereof, comprises 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor, or a pharmaceutically acceptable salt thereof, as defined herein, together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents.

Thus, the present invention further provides formulations, as defined above, and methods of making a pharmaceutical composition comprising admixing 4-(4-fluoro-2-methyl-1H-indol-5-yloxy)-6-methoxy-7-(3-(pyrrolidin-1-yl)propoxy)quinazoline, or a pharmaceutically acceptable salt thereof, or a mTOR-selective kinase inhibitor, or a pharmaceutically acceptable salt thereof, together with one or more pharmaceutically acceptable carriers, excipients, buffers, adjuvants, stabilisers, or other materials, as described herein.

The term “pharmaceutically acceptable” as used herein pertains to compounds, materials, compositions, and/or dosage forms which are, within the scope of sound medical judgement, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Suitable carriers, diluents, excipients, etc. can be found in standard pharmaceutical texts. See, for example, Handbook of Pharmaceutical Additives, 2nd Edition (eds. M. Ash and I. Ash), 2001 (Synapse Information Resources, Inc., Endicott, N.Y., USA); Remington's Pharmaceutical Sciences, 20th edition, pub. Lippincott, Williams & Wilkins, 2000 or Handbook of Pharmaceutical Excipients, 2nd edition, 1994.

The formulations may conveniently be presented in unit dosage form and may be prepared by any methods well known in the art of pharmacy. Such methods include the step of bringing into association the active compound with the carrier which constitutes one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, lozenges, granules, powders, capsules, cachets, pills, ampoules, suppositories, pessaries, ointments, gels, pastes, creams, sprays, mists, foams, lotions, oils, boluses, electuaries, or aerosols.

Formulations suitable for oral administration (e.g., by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g. compression or molding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g., sodium lauryl sulfate); and preservatives (e.g., methyl p-hydroxybenzoate, propyl p-hydroxybenzoate, sorbic acid). Molded tablets may be made by molding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Formulations suitable for topical administration (e.g. transdermal, intranasal, ocular, buccal, and sublingual) may be formulated as an ointment, cream, suspension, lotion, powder, solution, past, gel, spray, aerosol, or oil. Alternatively, a formulation may comprise a patch or a dressing such as a bandage or adhesive plaster impregnated with active compounds and optionally one or more excipients or diluents.

Formulations suitable for topical administration in the mouth include losenges comprising the active compound in a flavored basis, usually sucrose and acacia or tragacanth; pastilles comprising the active compound in an inert basis such as gelatin and glycerin, or sucrose and acacia; and mouthwashes comprising the active compound in a suitable liquid carrier.

Formulations suitable for topical administration to the eye also include eye drops wherein the active compound is dissolved or suspended in a suitable carrier, especially an aqueous solvent for the active compound.

Formulations suitable for nasal administration, wherein the carrier is a solid, include a coarse powder having a particle size, for example, in the range of about 20 to about 500 microns which is administered in the manner in which snuff is taken, i.e. by rapid inhalation through the nasal passage from a container of the powder held close up to the nose. Suitable formulations wherein the carrier is a liquid for administration as, for example, nasal spray, nasal drops, or by aerosol administration by nebuliser, include aqueous or oily solutions of the active compound.

Formulations suitable for administration by inhalation include those presented as an aerosol spray from a pressurised pack, with the use of a suitable propellant, such as dichlorodifluoromethane, trichlorofluoromethane, dichloro-tetrafluoroethane, carbon dioxide, or other suitable gases.

Formulations suitable for topical administration via the skin include ointments, creams, and emulsions. When formulated in an ointment, the active compound may optionally be employed with either a paraffinic or a water-miscible ointment base. Alternatively, the active compounds may be formulated in a cream with an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues.

When formulated as a topical emulsion, the oily phase may optionally comprise merely an emulsifier (otherwise known as an emulgent), or it may comprises a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulgents and emulsion stabilisers include Tween 60, Span 80, cetostearyl alcohol, myristyl alcohol, glyceryl monostearate and sodium lauryl sulphate. The choice of suitable oils or fats for the formulation is based on achieving the desired cosmetic properties, since the solubility of the active compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active compound, such carriers as are known in the art to be appropriate.

Formulations suitable for parenteral administration (e.g., by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

The size of the dose of each therapy which is required for the therapeutic or prophylactic treatment of a particular disease state will necessarily be varied depending on the host treated, the route of administration and the severity of the illness being treated. Accordingly the optimum dosage may be determined by the practitioner who is treating any particular patient, and taking into consideration various factors known to modify the action of drugs including severity and type of disease, body weight, sex, diet, time and route of administration, other medications and other relevant clinical factors. It may also be necessary or desirable to reduce the above-mentioned doses of the components of the combination treatments in order to reduce toxicity. Therapeutically effective dosages may be determined by either in vitro or in vivo methods.

The compositions described herein may be in a form suitable for oral administration, for example as a tablet or capsule, for nasal administration or administration by inhalation, for example as a powder or solution, for parenteral injection (including intravenous, subcutaneous, intramuscular, intravascular or infusion) for example as a sterile solution, suspension or emulsion, for topical administration for example as an ointment or cream, for rectal administration for example as a suppository or the route of administration may be by direct injection into the tumour or by regional delivery or by local delivery. In other embodiments of the present invention the 4-(4-fluoro-2-methyl-1H-indol-5-yloxy)-6-methoxy-7-(3-(pyrrolidin-1-yl)propoxy)quinazoline, or a pharmaceutically acceptable salt thereof, or the mTOR-selective kinase inhibitor, or a pharmaceutically acceptable salt thereof, of the combination treatment may be delivered endoscopically, intratracheally, intralesionally, percutaneously, intravenously, subcutaneously, intraperitoneally or intratumourally. Preferably 4-(4-fluoro-2-methyl-1H-indol-5-yloxy)-6-methoxy-7-(3-(pyrrolidin-1-yl)propoxy)quinazoline, or a pharmaceutically acceptable salt thereof, is administered orally. Preferably the mTOR-selective kinase inhibitor, or a pharmaceutically acceptable salt thereof, is administered orally. In general the compositions described herein may be prepared in a conventional manner using conventional excipients. The compositions of the present invention are advantageously presented in unit dosage form.

4-(4-fluoro-2-methyl-1H-indol-5-yloxy)-6-methoxy-7-(3-(pyrrolidin-1-yl)propoxy)quinazoline, or a pharmaceutically acceptable salt thereof, will normally be administered to a warm-blooded animal at a unit dose within the range 1-50 mg per square metre body area of the animal, for example approximately 0.03-1.5 mg/kg in a human. A unit dose in the range, for example, 0.01-1.5 mg/kg, preferably 0.03-0.5 mg/kg is envisaged and this is normally a therapeutically-effective dose. A unit dosage form such as a tablet or capsule will usually contain, for example 1-50 mg of active ingredient. Preferably a daily dose in the range of 0.03-0.5 mg/kg is employed.

A mTOR-selective kinase inhibitor, or a pharmaceutically acceptable salt thereof, will normally be administered to a warm-blooded animal so that a daily dose in the range, for example, 0.01 mg/kg to 75 mg/kg body weight is received, given, if required, in divided doses. A mTOR-selective kinase inhibitor, or a pharmaceutically acceptable salt thereof, may be administered orally such as in a tablet or capsule. A mTOR-selective kinase inhibitor, or a pharmaceutically acceptable salt thereof, may also be administered parenterally. In such cases lower doses will be used. Thus, for example, for intravenous administration, a dose in the range 0.01 mg/kg to 30 mg/kg body weight will generally be used.

The dosages and schedules may vary according to the particular disease state and the overall condition of the patient. Dosages and schedules may also vary if, in addition to a combination treatment of the present invention, one or more additional chemotherapeutic agents is/are used. Scheduling can be determined by the practitioner who is treating any particular patient.

The following terms, unless otherwise indicated, shall be understood to have the following meanings.

A small molecular weight compound refers to a compound with a molecular weight of less than 2000 Daltons, 1000 Daltons, 700 Daltons or 500 Daltons.

A patient is any warm-blooded animal, such as a human.

The term treatment includes therapeutic and/or prophylactic treatment.

The invention will now be illustrated by the following non-limiting examples, which are provided for illustrative purposes only and are not to be construed as limiting upon the teachings herein, in which:

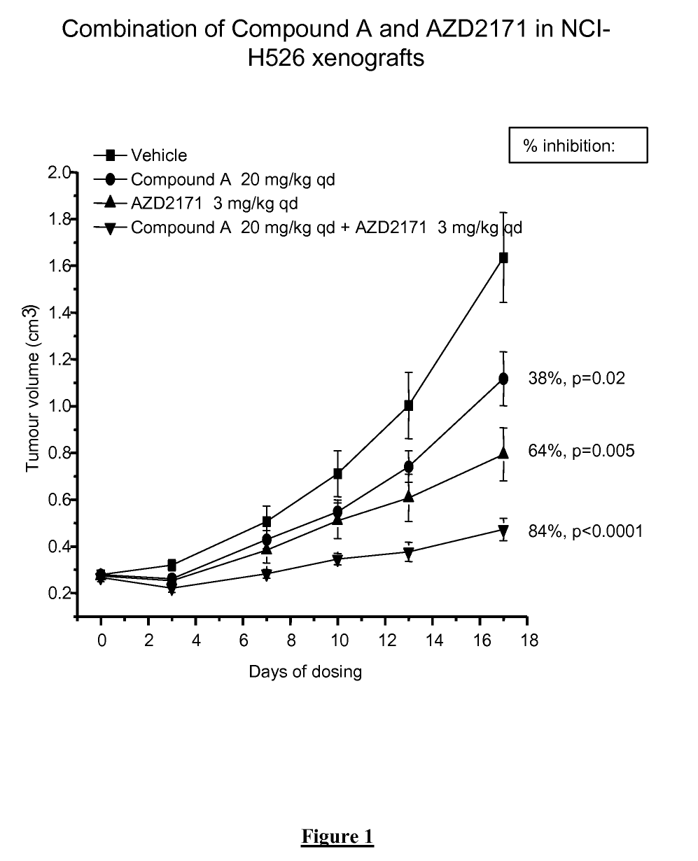

FIG. 1. Combination of AZD2171 and Compound A in NCI-526 xenografts; tumour volume in cm³ against days of dosing. Squares represent vehicle; circles represent Compound A monotherapy; triangles represent AZD2171 monotherapy; inverted triangles represent Compound A and AZD2171 in combination.

### EXAMPLES

### General Experimental Methods

Thin Layer chromatography was carried out using Merck Kieselgel 60 F₂₅₄ glass backed plates. The plates were visualized by the use of a UV lamp (254 nm). Silica gel 60 (particle sizes 40-63 □m) supplied by E. M. Merck was employed for flash chromatography. ¹H NMR spectra were recorded at 300 MHz on a Bruker DPX-300 instrument. Chemical shifts were referenced relative to tetramethylsilane.

### Purification of Samples

The samples were purified on Gilson LC units. Mobile phase A—0.1% aqueous TFA, mobile phase B—Acetonitrile; flow rate 6 ml/min; Gradient—typically starting at 90% A/10% B for 1 minute, rising to 97% after 15 minutes, holding for 2 minutes, then back to the starting conditions. Column: Jones Chromatography Genesis 4 □m, C18 column, 10 mm×250 mm. Peak acquisition based on UV detection at 254 nm.

### Identification of Samples

Mass spectra were recorded on a Finnegan LCQ instrument in positive ion mode. Mobile phase A—0.1% aqueous formic acid. Mobile phase B—Acetonitrile; Flowrate 2 ml/min; Gradient—starting at 95% A/5% B for 1 minute, rising to 98% B after 5 minutes and holding for 3 minutes before returning to the starting conditions. Column: Varies, but always C18 50 mm×4.6 mm (currently Genesis C18 4 □m. Jones Chromatography). PDA detection Waters 996, scan range 210-400 nm.

### QC Method QC2-Long

Mass spectra were recorded on a Waters ZQ instrument in Electrospray ionisation mode. Mobile phase A—0.1% aqueous formic acid. Mobile phase B—0.1% Formic acid in acetonitrile; Flowrate 2 ml/min; Gradient—starting at 95% A/5% B, rising to 95% B after 20 minutes and holding for 3 minutes before returning to the starting conditions. Column: Varies, but always C18 50 mm×4.6 mm (currently Genesis C18 4 u 50 mm×4.6 mm, Hichrom Ltd). PDA detection Waters 996, scan range 210-400 nm.

### Microwave Synthesis

Reactions were carried out using a Personal Chemistry™ Emrys Optimiser microwave synthesis unit with robotic arm. Power range between. 0-300 W at 2.45 GHz. Pressure range between 0-20 bar; temperature increase between 2-5° C./sec; temp range 60-250° C.

### General Procedure for the Synthesis of 2,4,7-Substituted Pyridopyrimidine Derivatives:

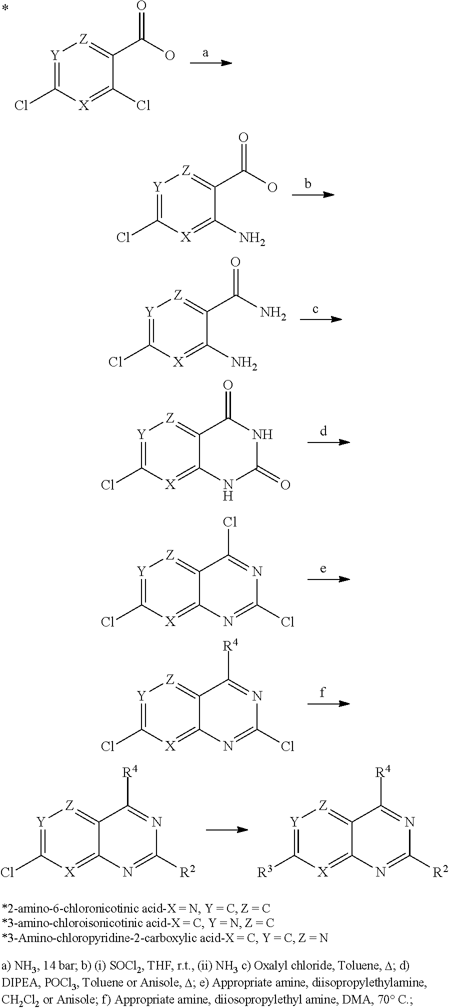

### Synthesis of 2,4,7-Substituted Pyridopyrimidine Derivatives

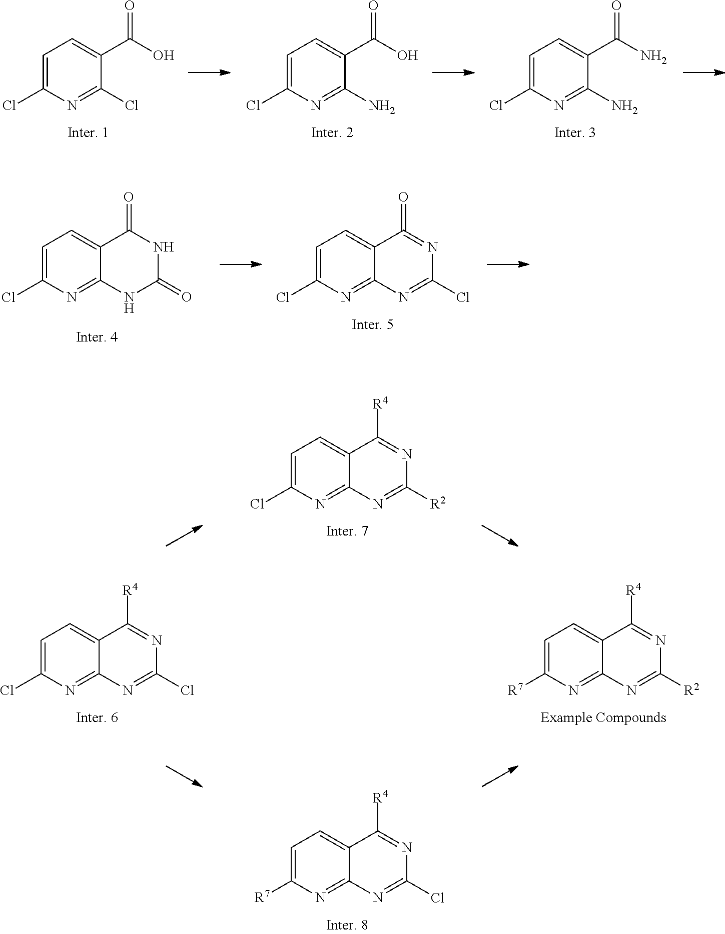

### Intermediates:

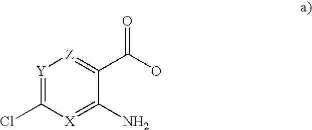

To the appropriate amino acid (1 equiv) was added liquid ammonia (sufficient to make a 0.6M solution of substrate in ammonia). The suspension was sealed in a pressure vessel which was then heated slowly to 130° C. It was noted that at this temperature a pressure of 18 bar was observed. This temperature and pressure was maintained for a further 16 hours whereupon the mixture was cooled to room temperature. The pressure vessel was opened and the reaction poured into ice cold water (1 reaction volume). The resulting solution was acidified to pH 1-2 using concentrated HCl which caused a precipitate to form. The acidic mixture was allowed to warm to room temperature and was stirred like this for a further 30 min The suspension was then extracted with diethyl ether (3×400 ml). The combined organic extracts were then filtered and the filtrate concentrated in vacuo to give a white solid which was dried further over P₂O₅ to give the title compound (typically 80-90% yield and 90%+pure) in suitably pure form to be used without any further purification.

### 2-Amino-6-chloronicotinic acid (Inter. 2)

To 2,6-dichloronicotinic acid (Inter. 1) (1 equiv) was added liquid ammonia (sufficient to make a 0.6M solution of substrate in ammonia). The suspension was sealed in a pressure vessel which was then heated slowly to 130° C. It was noted that at this temperature a pressure of 18 bar was observed. This temperature and pressure was maintained for a further 16 hours whereupon the mixture was cooled to room temperature. The pressure vessel was opened and the reaction poured into ice cold water (1 reaction volume). The resulting solution was acidified to pH 1-2 using concentrated HCl which caused a precipitate to form. The acidic mixture was allowed to warm to room temperature and was stirred like this for a further 30 minutes. The suspension was then extracted with diethyl ether (3×400 ml). The combined organic extracts were then filtered and the filtrate concentrated in vacuo to give a white solid which was dried further over P₂O₅ to give the title compound (90% yield and 96% pure) in suitably pure form to be used without any further purification. m/z (LC-MS, ESP): 173 [M+H]⁺ R/T=3.63 mins

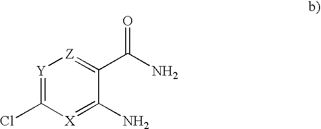

To a 0.3 M solution of amino acid (1 equiv) in anhydrous THF, under an inert atmosphere, was added thionyl chloride (3.3 equiv) in a dropwise fashion. The reaction mixture was stirred at room temperature for 2 hours. After this time the reaction was concentrated in vacuo to give a crude yellow solid residue. The crude solid was dissolved in THF (equal to initial reaction volume) and concentrated in vacuo again to give a yellow solid residue. The residue was dissolved once more in THF and concentrated as before to give a solid residue which was then dissolved in THF (to give a solution of 0.3M) and ammonia gas bubbled through the solution for 1 hour. The resultant precipitate was removed by filtration and the filtrate concentrated in vacuo to give a yellow precipitate which was triturated with water at 50° C. then dried to give the title compound (typically 90-95%) yield and suitably clean enough to be used without any further purification.

### 2-Amino-6-chloronicotinamide (Inter. 3)

To a 0.3 M solution of 2-amino-6-chloronicotinic acid (Inter. 2) (1 equiv) in anhydrous THF, under an inert atmosphere, was added thionyl chloride (3.3 equiv) in a dropwise fashion. The reaction mixture was stirred at room temperature for 2 hours. After this time the reaction was concentrated in vacuo to give a crude yellow solid residue. The crude solid was dissolved in THF (equal to initial reaction volume) and concentrated in vacuo again to give a yellow solid residue. The residue was dissolved once more in THF and concentrated as before to give a solid residue which was then dissolved in THF (to give a solution of 0.3M) and ammonia gas bubbled through the solution for 1 hour. The resultant precipitate was removed by filtration and the filtrate concentrated in vacuo to give a yellow precipitate which was triturated with water at 50° C. then dried to give the title compound (92% yield, 93% purity), suitably clean to be used without any further purification. m/z (LC-MS, ESP): 172 [M+H]⁺ R/T=3.19 mins

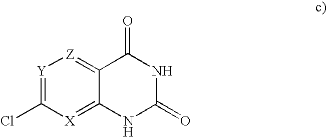

To a stirred solution (0.06 M) of substrate (1 equiv) in anhydrous toluene under an inert atmosphere was added oxalyl chloride (1.2 equiv) in a dropwise manner. The resulting mixture was then heated to reflux (115° C.) for 4 hours whereupon it was cooled and stirred for a further 16 hours. The crude reaction mixture was then concentrated to half its volume in vacuo and filtered to give the desired product in suitably pure form to be used without any further purification.

### 7-Chloro-1H-pyrido[2,3-d]pyrimidine-2,4-dione (Inter. 4)

To a stirred solution (0.06 M) of 2-amino-6-chloronicotinamide (Inter. 3) (1 equiv) in anhydrous toluene under an inert atmosphere was added oxalyl chloride (1.2 equiv) in a dropwise manner. The resulting mixture was then heated to reflux (115° C.) for 4 hours whereupon it was cooled and stirred for a further 16 hours. The crude reaction mixture was then concentrated to half its volume in vacuo and filtered to give the desired product in suitably pure form (95% yield, 96% purity) to be used without any further purification. m/z (LC-MS, ESP): 196 [M−H]⁻ R/T=3.22 mins

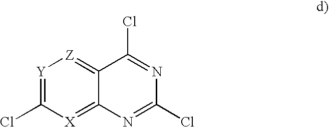

To a stirred 0.5 M suspension of the appropriate dione (1 equiv) in anhydrous toluene under an inert atmosphere was slowly added diisopropylethylamine (3 equiv). The reaction mixture was then heated to 70° C. for 30 minutes and then cooled to room temperature prior to the addition of POCl₃ (3 equiv). The reaction was then heated to 100° C. for 2.5 hours before being cooled and concentrated in vacuo to give a crude slurry which was then suspended in EtOAc and filtered through a thin pad of Celite™. The filtrate was concentrated in vacuo to give a brown, oil which was dissolved in CH₂Cl₂ and stirred over silica gel for 30 minutes. After this time the silica was removed by filtration, the filtrate concentrated and the crude residue purified by flash chromatography (SiO₂) to give the title compound in analytically pure form.

### 2,4,7-Trichloro-pyrido[2,3-d]pyrimidine (Inter. 5)

To a stirred 0.5 M suspension of the dione (Inter. 4) (1 equiv.) in anhydrous toluene under an inert atmosphere was slowly added diisopropylethylamine (3 equiv.). The reaction mixture was then heated to 70° C. for 30 minutes and then cooled to room temperature prior to the addition of POCl₃ (3 equivalents). The reaction was then heated to 100° C. for 2.5 hours before being cooled and concentrated in vacuo to give a crude slurry which was then suspended in EtOAc and filtered through a thin pad of Celite™. The filtrate was concentrated in vacuo to give a brown, oil which was dissolved in CH₂Cl₂ and stirred over silica gel for 30 minutes. After this time the silica was removed by filtration, the filtrate concentrated and the crude residue purified by flash chromatography (SiO₂) to give the title compound in analytically pure form (48% yield, 96% purity). m/z (LC-MS, ESP): 234 [M+H]⁺ R/T=4.21 mins

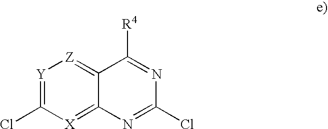

To a cooled (0-5° C.) stirred solution (0.1 M) of the appropriate trichloro-substrate (1 equiv) in CH₂Cl₂ was added diisopropylethylamine (1 equiv) in a dropwise fashion. The appropriate amine (1 equiv) was then added to the reaction mixture portionwise over the period of 1 hour. The solution was maintained at room temperature with stirring for a further 1 hour before the mixture was washed with water (2×1 reaction volume). The aqueous extracts were combined and extracted with CH₂Cl₂ (2×1 reaction volume). The organic extracts were then combined, dried (sodium sulphate), filtered and concentrated in vacuo to give an oily residue which solidified upon prolonged drying. The solid was triturated with diethylether and then filtered and the cake washed with cold diethyl ether to leave the title compound in suitable clean form to be used without any further purification.

### 4-Amino-2,7-dichloropyridopyrimidines (Inter. 6)

To a cooled (0-5° C.) stirred solution (0.1 M) of the trichloro substrate (Inter. 5) (1 equiv.) in CH₂Cl₂ was added diisopropylethylamine (1 equiv.) in a dropwise fashion. The appropriate amine (1 equiv.) was then added to the reaction mixture portionwise over the period of 1 hour. The solution was maintained at room temperature with stirring for a further 1 hour before the mixture was washed with water (2×1 reaction volume). The aqueous extracts were combined and extracted with CH₂Cl₂ (2×1 reaction volume). The organic extracts were then combined, dried (sodium sulphate), filtered and concentrated in vacuo to give an oily residue which solidified upon prolonged drying. The solid was triturated with diethylether and then filtered and the cake washed with cold diethyl ether to leave the title compound in suitable clean form to be used without any further purification.

Inter. 6a: 2,7-Dichloro-4-morpholin-4-yl-pyrido[2,3-d]pyrimidine; R⁴=morpholino; (92% yield, 90% purity) m/z (LC-MS, ESP): 285 [M+H]⁺ R/T=3.90 mins
Inter. 6b: 2,7-Dichloro-4-((2S,6R)-2,6-dimethyl-morpholin-4-yl)-pyrido[2,3-d]pyrimidine; R⁴=(2R,6S)-2,6-Dimethyl-morpholino; (99% yield, 90% purity) m/z (LC-MS, ESP): 313 [M+H]⁺ R/T=4.39 mins
Inter. 6c(i): 2,7-Dichloro-4-((S)-3-methyl-morpholin-4-yl)-pyrido[2,3-d]pyrimidine—R⁴=(S)-3-Methyl-morpholine, X═N, Y═C, Z═C: (87% yield, 92% purity) m/z (LC-MS, ESP): 301 [M+H]⁺ R/T=4.13 min
Inter. 6c(ii): 2,7-Dichloro-4-((R)-3-methyl-morpholin-4-yl)-pyrido[2,3-d]pyrimidine—R⁴=(R)-3-Methyl-morpholine: (99% yield, 94% purity) m/z (LC-MS, ESP): 301 [M+H]⁺ R/T=3.49 min

Alternatively, to a stirred 0.47 M suspension of the appropriate dione (1 equiv) in anhydrous anisole under an inert atmosphere was added POCl₃ (2.6 equiv). The mixture was heated to 55° C. and then diisopropylethylamine (2.6 equiv) was slowly added. The reaction mixture was then heated to 85-90° C. for 30 minutes. Water was added in portions (0.15 equiv), and the reaction mixture was held at 85-90° C. for a further 30 minutes. The reaction was cooled to 50° C., and then 15% of the anisole solvent was removed by vacuum distillation. The mixture was then cooled to −5° C. and diisopropylethylamine (1.1 equiv) was added. A 4.9M solution of the appropriate amine (1.05 equiv) in anisole was then added to the reaction mixture continuously over a period of 1 hour. The solution was then warmed to 30° C. and the reaction monitored by HPLC until reaction completion.

One third of the resulting mixture from the above reaction was then added over 10 min to a stirred mixture of 1.95M aqueous potassium hydroxide (3.9 equiv) and i-butanol (6.9 equiv) at 60° C. The stirring was stopped, the phases were allowed to separate, and the aqueous phase was removed. Stirring was resumed, and 1.95M aqueous potassium hydroxide (3.9 equiv) was added to the retained organic phase. The second third of the resulting reaction mixture from the reaction above was then added over 10 min at 60° C. Again, stirring was stopped, the phases were allowed to separate, and the aqueous phase was removed. Stirring was resumed, and 1.95M aqueous potassium hydroxide (3.9 equiv) was added to the retained organic phase. The remaining third of the resulting reaction mixture from the reaction above was then added over 10 min at 60° C. Again, stirring was stopped, the phases were allowed to separate, and the aqueous phase was removed. Water was then added to the organic phase with stirring, and the stirred mixture heated to 75° C. Stirring was stopped, the phases were allowed to separate, and the aqueous phase was removed. The resulting organic phase was stirred and allowed to cool to 30° C., and then as the mixture was heated to 60° C. heptane (11.5 equiv) was added over 20 min when the mixture was around 40° C. After being heated to 60° C., the mixture was cooled over 2.5 h to 10° C. After 30 min, the resulting slurry was filtered off, washed with a 10:1 heptane:anisole mixture (2×1.4 equiv) and then washed with heptane (2×1.4 equiv). The solid was then dried in a vacuum oven at 50° C. to leave the title compound in suitable clean form to be used without any further purification.

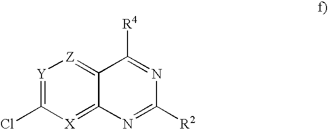

To a solution (0.2 M) of the appropriate dichloro-substrate (1 equiv) in anhydrous dimethyl acetamide under an inert atmosphere was added diisopropylethylamine (1 equiv) followed by the appropriate amine (1 equiv). The resulting mixture was heated for 48 hours at 70° C. before being cooled to ambient temperature. The reaction was diluted with CH₂Cl₂ (1 reaction volume) and then washed with water (3×1 reaction volumes). The organic extract was concentrated in vacuo to give a syrup which was dissolved in EtOAc (1 reaction volume) and washed with saturated brine solution before being dried (sodium sulphate) and concentrated in vacuo to give an oil. The crude residue was purified by flash chromatography (SiO₂, eluted with EtOAc:Hex (7:3) going to (1:1)) to give the title compound as a yellow solid that was suitably clean to be used without any further purification.

### 2,4-Diamino-7-chloropyridopyrimidines (Inter. 7)

To a solution (0.2 M) of the appropriate dichloro-substrate (Inter. 6a, 6b, 6c(i) or 6c(ii)) (1 equiv) in anhydrous dimethyl acetamide under an inert atmosphere was added diisopropylethylamine (1 equiv) followed by the appropriate amine (1 equiv.). The resulting mixture was heated for 48 hours at 70° C. before being cooled to ambient temperature. The reaction was diluted with CH₂Cl₂ (1 reaction volume) and then washed with water (3×1 reaction volumes). The organic extract was concentrated in vacuo to give a syrup which was dissolved in EtOAC (1 reaction volume) and washed with saturated brine solution before being dried (sodium sulphate) and concentrated in vacuo to give an oil. The crude residue was purified by flash chromatography (SiO₂, eluted with EtOAc:Hex (7:3) going to (1:1)) to give the title compound as a yellow solid that was suitably clean to be used without any further purification.

Inter. 7a: 7-Chloro-2-((2S,6R)-2,6-dimethyl-morpholin-4-yl)-4-morpholin-4-yl-pyrido[2,3-d]pyrimidine; R⁴=morpholine, R²=cis-dimethylmorpholine; (45% yield, 85% purity) m/z (LC-MS, ESP): 348 [M+H]⁺ R/T=4.16 mins
Inter. 7b: 7-Chloro-4-(2-methyl-piperidin-1-yl)-2-morpholin-4-yl-pyrido[2,3-d]pyrimidine; R⁴=morpholine, R²=2-methylpiperidine; (57% yield, 95% purity) m/z (LC-MS, ESP): 348.1 [M+H]⁺ R/T=3.42 mins
Inter. 7c: 7-Chloro-4-((2S,6R)-2,6-dimethyl-morpholin-4-yl)-2-((S)-3-methyl-morpholin-4-yl)pyrido[2,3-d]pyrimidine (intermediate for compound 11k:) R⁴=cis-dimethylmorpholine, R²=(S)-3-Methyl-morpholine; (48% yield, 90% purity) m/z (LC-MS, ESP): 378 [M+H]⁺ R/T=3.74 mins
Inter. 7d: 7-Chloro-2-((S)-3-methyl-morpholin-4-yl)-4-morpholin-4-yl-pyrido[2,3-d]pyrimidine (Intermediate for compound 11a): R⁴=morpholine, R²=(S)-3-Methyl-morpholine; (70% yield, 97% purity) m/z (LC-MS, ESP): 350 [M+H]⁺ R/T=3.44 mins
Inter. 7e: 7-Chloro-2-(2-ethyl-piperidin-1-yl)-4-morpholin-4-yl-pyrido[2,3-d]pyrimidine (intermediate for compound 11a): R⁴=morpholine, R²=2-Ethyl-piperidine; (56% yield, 95% purity) m/z (LC-MS, ESP): 362 [M+H]⁺ R/T=3.78 mins
Inter. 7f: 7-Chloro-4-((S)-3-methyl-morpholin-4-yl)-2-((S)-3-methyl-morpholin-4-yl)-pyrido[2,3-d]pyrimidine—R⁴=(S)-3-Methyl-morpholine, R²=(S)-3-Methyl-morpholine, X═N, Y═C, Z═C: (71% yield, 90% purity) m/z (LC-MS, ESP): 364 [M+H]⁺ R/T=3.52 min
Inter. 7g: 7-Chloro-2-(2-ethyl-piperidin-1-yl)-4-((S)-3-methyl-morpholin-4-yl)-pyrido[2,3-d]pyrimidine—R⁴=(S)-3-Methyl-morpholine, R²=2-Ethyl-piperidine, X═N, Y═C, Z═C: (51% yield, 98% purity) m/z (LC-MS, ESP): 376 [M+H]⁺ R/T=3.88 min
Inter. 7h: 7-Chloro-4-((S)-3-methyl-morpholin-4-yl)-2-morpholin-4-yl-pyrido[2,3-d]pyrimidine, —R⁴=(S)-3-Methyl-morpholine, R²=morpholine, X═N, Y═C, Z═C: (72% yield, 96% purity) m/z (LC-MS, ESP): 350 [M+H]⁺ R/T=3.45 min
Inter. 7i: 7-Chloro-2-((2S,6R)-2,6-dimethyl-morpholin-4-yl)-4-((S)-3-methyl-morpholin-4-yl-pyrido[2,3-d]pyrimidine—R⁴=(S)-3-Methyl-morpholine, R²=cis-dimethylmorpholine: (33% yield) m/z (LC-MS, ESP): 378 [M+H]⁺ R/T=3.68 min
Inter. 7j: 7-Chloro-4-((R)-3-methyl-morpholin-4-yl)-2-((R)-3-methyl-morpholin-4-yl)-pyrido[2,3-d]pyrimidine, —R⁴=R²=(R)-3-Methyl-morpholine: (48% yield, 100% purity) m/z (LC-MS, ESP): 364 [M+H]⁺ R/T=2.80 min

To a 0.33 M solution of 2,7-dichloro-4-((S)-3-methyl-morpholin-4-yl)-pyrido[2,3-d]pyrimidine (1 equiv) in N,N-dimethylacetamide was added Hunig's base (1 equiv) followed by the appropriate amine (1.1 equiv). The reaction mixture was heated 40° C. for 1 hour. After this time the reaction was allowed to cool, diluted with EtOAc (1 reaction volume) and then washed with water (1 reaction volume). The aqueous fraction was removed and extracted further with EtOAc (2×1 reaction volume). The combined organic extracts were dried (MgSO₄), filtered and concentrated in vacuo to give a crude oily residue which was purified by flash chromatography (SiO₂) using EtOAc/Hexanes as eluent which furnished the desired products in a suitably clean form.

Inter. 7k: 7-Chloro-4-((S)-3-methyl-morpholin-4-yl)-2-thiomorpholin-4-yl-pyrido[2,3-d]pyrimidine: (30% yield, 100% purity) m/z (LC-MS, ESP): 366.4 [M+H]⁺ R/T=3.00 min
Inter. 7l: 7-Chloro-4-((S)-3-methyl-morpholin-4-yl)-2-(4-methyl-piperazin-1-yl)-pyrido[2,3-d]pyrimidine: (32% yield, 95% purity) m/z (LC-MS, ESP): 363.4 [M+H]⁺ R/T=2.37 min

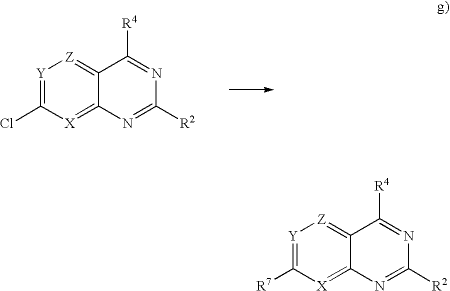

The appropriate chloro-substrate (1 equiv) was dissolved in a toluene/ethanol (1:1) solution (0.02 M). Sodium carbonate (2 equiv) and the appropriate pinacolate boron ester or boronic acid (1 equiv) were then added followed by tetrakis(triphenylphosphine) palladium⁰ (0.1 equiv). The reaction vessel was sealed and the mixture exposed to microwave radiation (140° C., medium absorption setting) for 30 minutes. Upon completion the samples were filtered through a silica cartridge, washed with EtOAc and then concentrated in vacuo. The crude residue was then purified by preparative HPLC to give the desired products.

### 4-amino-7-aryl-2-chloropyridopyrimidines (Inter. 8)

To a solution (0.09 M) of the appropriate boronic acid or ester (1 equiv) in water (1 volume) was added the appropriate 2,7-dichloro-4-amino pyridopyrimidine (1 equiv) (Inter. 6a, 6b, 6c(i) or 6c(ii)) potassium carbonate (2.5 equiv) and acetonitrile (1 volume). The mixture was degassed by bubbling nitrogen through the solution while sonicating for 15 minutes before the addition of by tetrakis(triphenylphosphine) palladium (0.03 equiv). The mixture was degassed for a further 5 minutes before heating under an inter atmosphere at 95° C. for 2 hours. Upon completion, the reaction was cooled to room temperature and filtered under vacuum. The filtrate was concentrated in vacuo to give a solid residue which was dissolved in CH₂Cl₂ (1 volume) and washed with water (1 volume). The organic extract was then dried (MgSO₄), filtered and concentrated in vacuo to give an amorphous solid which was triturated with Et₂O to leave the desired product as a fine powder.

Inter. 8a (R⁴=Morpholine, R⁷=4-chlorophenyl) 2-Chloro-7-(4-chloro-phenyl)-4-morpholin-4-yl-pyrido[2,3-d]pyrimidine; ¹H NMR (300 MHz, Solvent CDCl₃?? δppm 8.29-7.96 (m, 2H), 7.75 (d, J=8.70 Hz, 1H), 7.54-7.21 (m, 2H), 5.29 (s, 1H), 3.91 (m, 8H).

### Example 1

### Preparation of 2,4,7-Substituted Pyridopyrimidine Intermediates

### Procedures for the Synthesis of 2-Chloro-4-((S)-3-methyl-morpholin-4-yl)-7-aryll-pyrido[2,3-d]pyrimidine Derivatives

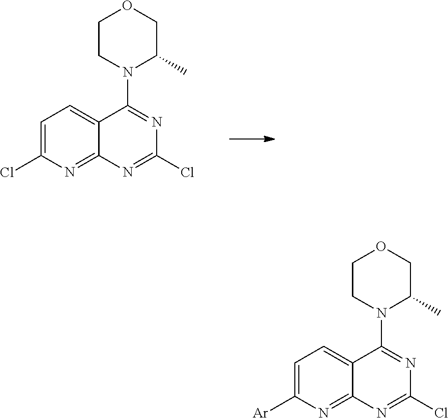

To a (0.1 M) solution of 2,7-dichloro-4-((S)-3-methyl-morpholin-4-yl)-pyrido[2,3-d]pyrimidine (1 equiv) in MeCN/H₂O (1:1 mixture) was added the appropriate pinacolate boron ester or boronic acid (1.1 equiv) and potassium carbonate (3 equiv). The mixture was degassed with nitrogen for 20 minutes before the addition of tetrakis(triphenylphosphine)palladium⁰ (0.05 equiv). The reaction was degassed for a further 5 minutes before being heated to reflux under an inert atmosphere for 3 hours. Whereupon, it was concentrated in vacuo and the crude residue partitioned between CH₂Cl₂/H₂O. The organic fraction was dried (MgSO₄), filtered and concentrated in vacuo to give an oil which was further purified by flash chromatography (SiO₂) using 5% MeOH in CH₂Cl₂ as eluent.

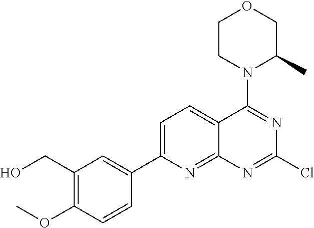

{5-[2-Chloro-4-((R)-3-methyl-morpholin-4-yl)-pyrido[2,3-d]pyrimidin-7-yl]-2-methoxy-phenyl}-methanol: (97% yield, 93% purity) m/z (LC-MS, ESP): 401 [M+H]⁺, R/T=3.42 min)

### General Procedures for the Synthesis of Boronic Ester:

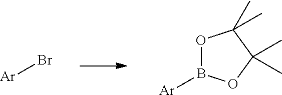

The bromo-aryl compound (1 equiv) was dissolved in dioxane (0.1 M). Bis(pinacolato)diboron (1.1 equiv), potassium acetate (3.5 equiv) and dppf (0.05 equiv) were added and the mixture was degassed with nitrogen for 20 minutes. (1,1′-Bis(diphenylphosphino)ferrocene-dichloropalladium (0.05 equiv) was added and the mixture was degassed for a further 5 minutes. The reaction mixture was heated to 120° C. for 2 hours under nitrogen. After cooling to room temperature, the reaction mixture was diluted with CH₂Cl₂ and filtered through Celite™. The filtrate was concentrated in vacuo to give a dark oil. The residue was partitioned between EtOAc and saturated aqueous sodium bicarbonate and the aqueous layer further extracted with EtOAc. The combined organic phases were dried (MgSO₄), filtered and the filtrate was concentrated in vacuo to give a residue. The residue may be purified by recrystallisation or may be purified by flash column chromatography for example on silica gel eluting with 0 to 30% ethyl acetate in hexane.

### Procedures for the Preparation of Examples 1a

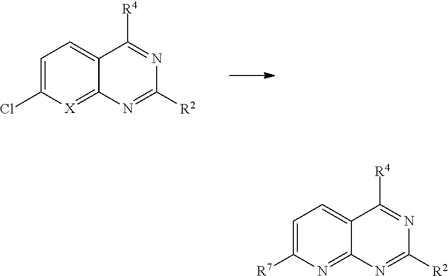 [0308] R⁴=(S)-3-methyl-morpholine
R²=(S)-3-methyl-morpholine or cis-dimethylmorpholine or 2-Ethyl-piperidine or morpholine or thiomorpholine or 4-methylpiperazine
R⁷=aryl or heteroaryl

### Procedures for the Suzuki Coupling:

The synthesis of the appropriate chloro-substrate has been described in the present document as intermediates. The appropriate pinacolate boron ester or boronic acids were prepared according to synthesis described in the present document (as intermediates) or commercially available, typically from the following suppliers:

### Sigma-Aldrich, Lancaster, Frontier Scientific, Boron Molecular, Interchim, Asymchem, Combi-blocks, Apollo Scientific, Fluorochem, ABCR, Digital Speciality Chemicals.

### Conditions A:

The appropriate chloro-substrate (1 equiv) was dissolved in a toluene/ethanol (1:1) solution (0.02 M). Sodium carbonate (2 equiv) and the appropriate pinacolate boron ester or boronic acid (1 equiv) were then added followed by tetrakis(triphenylphosphine) palladium⁰ (0.1 equiv). The reaction vessel was sealed and the mixture exposed to microwave radiation (140° C., medium absorption setting) for 30 minutes. Upon completion the samples were filtered through a silica cartridge, washed with EtOAc and then concentrated in vacuo. The crude residue was then purified by preparative HPLC to give the desired products.

### Conditions B:

A mixture of the appropriate chloro-substrate (1 equiv), potassium carbonate (2.4 equiv), the appropriate pinacolate boron ester or boronic acid (1.1 equiv) and tetrakis(triphenylphosphine) palladium⁰ (0.05 equiv) in n-butanol (0.03 M of chloro-substrate) was stirred at 120° C. for 2 hours. Upon completion the samples were filtered through a silica cartridge, washed through with CH₂Cl₂ and then concentrated in vacuo. The crude residue was then purified by preparative HPLC to give the desired products.

### Conditions C:

To a mixture of the appropriate chloro-substrate (1 equiv), potassium carbonate (2.4 equiv), and the appropriate pinacolate boron ester or boronic acid (1.1 equiv) in acetonitrile/water (1:1) (0.041 M of chloro-substrate) was added tetrakis(triphenylphosphine) palladium⁰ (0.05 equiv). The reaction vessel was sealed and exposed to microwave radiation (150° C., medium absorption setting) for 30 minutes under nitrogen atmosphere. Upon completion the samples were filtered through a silica cartridge, washed with CH₂Cl₂ and methanol and then concentrated in vacuo. The crude residue was then purified by preparative HPLC to give the desired products.

### Conditions D:

To a mixture of the appropriate chloro-substrate (1 equiv), potassium carbonate (1.2 equiv), and the appropriate pinacolate boron ester or boronic acid (1.2 equiv) in acetonitrile/water (1:1) (0.083 M of chloro-substrate) was added tetrakis(triphenylphosphine) palladium⁰ (0.05 equiv). The reaction vessel was sealed and exposed to microwave radiation (130° C., medium absorption setting) for 25 minutes under nitrogen atmosphere. Upon completion the sample was purified by column chromatography on silica gel using a gradient MeOH/CH₂Cl₂ to afford the desired product which was recrystallised from diethyl ether.

### Conditions E:

To a mixture of the appropriate chloro-substrate (1 equiv), potassium carbonate (2.4 equiv), and the appropriate pinacolate boron ester or boronic acid (1.3 equiv) in acetonitrile/water (1:1) (0.041 M of chloro-substrate) was added tetrakis(triphenylphosphine) palladium⁰ (0.05 equiv). The reaction vessel was sealed and heated at 95° C. for 16 hours. Upon completion the reaction mixture was partitioned between aqueous HCl and CH₂Cl₂ and washed with aqueous HCl. Combined aqueous phase were extracted with CH₂Cl₂ (2×), neutralised with aqueous NaOH (2N) to give a cloudy solution that was extracted with CH₂Cl₂. Combined organic phases were washed with brine, dried (MgSO₄), filtered and concentrated in vacuo. The crude residue was purified by column chromatography on silica gel eluting with 0 to 4% MeOH in CH₂Cl₂ to give the desired product.

### Conditions F:

To a mixture of the appropriate chloro-substrate (1 equiv), potassium carbonate (2.0 equiv), and the appropriate pinacolate boron ester or boronic acid (1.5 equiv) in acetonitrile/water (1:1) (0.028 M of chloro-substrate) was added tetrakis(triphenylphosphine) palladium⁰ (0.05 equiv). The reaction vessel was sealed and heated at 120° C. for 2 hours under nitrogen atmosphere. Upon completion the reaction mixture was partitioned between water and CH₂Cl₂ and extracted with CH₂Cl₂. Combined organic phases were dried (MgSO₄), filtered and concentrated in vacuo. The crude residue was purified by column chromatography on silica gel eluting with 0 to 4% MeOH in CH₂Cl₂ to give the desired product which was recrystallised from hexane/diethyl ether.

### Conditions G:

To a mixture of the appropriate chloro-substrate (1 equiv), potassium carbonate (3.0 equiv), and the appropriate pinacolate boron ester or boronic acid (1.05 equiv) in acetonitrile/water (1:1) (0.068 M of chloro-substrate) was added tetrakis(triphenylphosphine) palladium⁰ (0.05 equiv). The reaction vessel was sealed and heated at 100° C. for 5 hours under nitrogen atmosphere. Upon completion the reaction mixture was partitioned between brine and CH₂Cl₂ and extracted with CH₂Cl₂. Combined organic phases were dried (MgSO₄), filtered and concentrated in vacuo. The crude residue was purified by column chromatography on silica gel eluting with 0 to 4% MeOH in CH₂Cl₂ to give the desired products which were recrystallised from hexane/CH₂Cl₂.

### Conditions H:

A mixture of the appropriate chloro-substrate (1 equiv), potassium carbonate (3.0 equiv), the appropriate pinacolate boron ester or boronic acid (1.1 equiv) and tetrakis(triphenylphosphine) palladium⁰ (0.05 equiv) in acetonitrile/water (0.1 M of chloro-substrate) was stirred at 100° C. for 8 hours. Upon completion the sample was concentrated in vacuo. The crude residue was then purified by preparative HPLC to give the desired product.

### Conditions I:

Conditions I were similar to conditions H apart form the heating method: 100° C. for 2 hours.

### Conditions J:

A mixture of the appropriate chloro-substrate (1 equiv), potassium carbonate (1.2 equiv), the appropriate pinacolate boron ester or boronic acid (1.2 equiv) and tetrakis(triphenylphosphine) palladium⁰ (0.05 equiv) in acetonitrile/water (0.03 M of chloro-substrate) was stirred at 100° C. for 2 hours. Upon completion the sample was concentrated in vacuo. The crude residue was then purified by preparative HPLC to give the desired product.

### Conditions K:

Conditions K were similar to conditions G apart form the heating method: 100° C. for 16 hours.

### Conditions L:

To a mixture of the appropriate chloro-substrate (1 equiv), potassium carbonate (2.5 equiv), and the appropriate pinacolate boron ester or boronic acid (1.10 equiv) in acetonitrile/water (1:1) (0.041 M of chloro-substrate) was added tetrakis(triphenylphosphine) palladium⁰ (0.05 equiv). The reaction vessel was sealed and exposed to microwave radiation (100° C., medium absorption setting) for 90 minutes. Upon completion the reaction mixture was partly concentrated. The residue was partitioned between water and ethyl acetate and extracted with ethyl acetate and n-butanol. Combined organic phases were dried (MgSO₄), filtered and concentrated in vacuo. The crude residue was purified by column chromatography on silica gel eluting with 30 to 10% hexane in ethyl acetate to give the desired product which was recrystallised from hexane/CH₂Cl₂.

### Conditions M:

A mixture of the appropriate chloro-substrate (1 equiv), cesium fluoride (3.0 equiv), the appropriate pinacolate boron ester or boronic acid (1.1 equiv) and tetrakis(triphenylphosphine) palladium⁰ (0.05 equiv) in acetonitrile/water (0.09 M of chloro-substrate) was stirred at 115° C. for 48 hours. Upon completion the sample was concentrated in vacuo to half original volume. The residue was partitioned between water and CH₂Cl₂. Organic phase was dried (MgSO₄), filtered and concentrated in vacuo. The crude residue was purified by column chromatography on silica gel eluting with 0 to 100% ethyl acetate in hexane to give the desired product.

### Conditions N:

A mixture of the appropriate chloro-substrate (1 equiv), tripotassium phosphate (1.5 equiv), the appropriate pinacolate boron ester or boronic acid (1.05 equiv) and bis(tri-t-butylphosphine) palladium (0.05 equiv) was suspended in dioxane (0.16 M of chloro-substrate). The reaction vessel was sealed and exposed to microwave radiation (170° C., medium absorption setting) for 45 minutes. Upon completion the sample was concentrated in vacuo. The residue was partitioned between water and CH₂Cl₂. The organic phase was dried (MgSO₄), filtered and concentrated in vacuo. The crude residue was purified by column chromatography on silica gel eluting with 40 to 100% ethyl acetate in hexane to give the desired product.

### Conditions O:

A mixture of the appropriate chloro-substrate (1 equiv), potassium carbonate (2.5 equiv), the appropriate pinacolate boron ester or boronic acid (1.1 equiv) and tetrakis(triphenylphosphine) palladium⁰ (0.05 equiv) in n-butanol (0.068 M of chloro-substrate) was stirred at 95° C. for 15 minutes. Upon completion, the residue was partitioned between ethyl acetate and brine. Organic phase was dried (MgSO₄), filtered and concentrated in vacuo. The crude residue was purified by column chromatography on silica gel eluting with 30 to 100% ethyl acetate in hexane to give the desired product which was recrystallised from ethyl acetate/hexane.

### Conditions P:

To a mixture of the appropriate chloro-substrate (1 equiv), potassium carbonate (2.0 equiv), and the appropriate pinacolate boron ester or boronic acid (2.0 equiv) in acetonitrile/water (1:1) (0.041 M of chloro-substrate) was added tetrakis(triphenylphosphine) palladium⁰ (0.05 equiv). The reaction vessel was sealed and exposed to microwave radiation (120° C., medium absorption setting) for 10 minutes under nitrogen atmosphere. Upon completion the samples were filtered through a silica cartridge, washed through with CH₂Cl₂ and the concentrated in vacuo. The crude residue was then purified by preparative HPLC to give the desired product.

### Conditions Q:

A mixture of the appropriate chloro-substrate (1 equiv), potassium carbonate (2.5 equiv), the appropriate pinacolate boron ester or boronic acid (1.1 equiv) and tetrakis(triphenylphosphine) palladium⁰ (0.05 equiv) were dissolved in n-butanol (0.056 M of chloro-substrate). The reaction vessel was sealed and exposed to microwave radiation (150° C., medium absorption setting) for 30 minutes. Upon completion the samples were filtered through a silica cartridge, washed with CH₂Cl₂ and methanol and then concentrated in vacuo. The crude residue was purified by column chromatography on silica gel eluting with ethyl acetate and then 5% MeOH in CH₂Cl₂ to give the desired product.

### Conditions R:

A mixture of the appropriate chloro-substrate (1 equiv), potassium carbonate (2.5 equiv), the appropriate pinacolate boron ester or boronic acid (1.2 equiv) and tetrakis(triphenylphosphine) palladium⁰ (0.05 equiv) in acetonitrile/water (0.05 M of chloro-substrate) was stirred at 115° C. for 1.5 hours. Upon completion the crude reaction was filtered and the filtrate was concentrated in vacuo. The crude residue was purified by column chromatography on silica gel eluting with 5 to 20% MeOH in CH₂Cl₂ to give the desired product.

### Conditions S:

A mixture of the appropriate chloro-substrate (1 equiv), potassium carbonate (10.0 equiv), the appropriate pinacolate boron ester or boronic acid (1.2 equiv) and tetrakis(triphenylphosphine) palladium⁰ (0.05 equiv) in acetonitrile/water (0.1 M of chloro-substrate) was stirred at 100° C. for 2 hours. Upon completion the reaction mixture was partitioned between water and CH₂Cl₂ and extracted with CH₂Cl₂. Combined organic phases were dried (MgSO₄), filtered and concentrated in vacuo. The crude residue was purified by column chromatography on silica gel eluting with 0 to 5% MeOH in CH₂Cl₂ to give the desired product which was recrystallised from hexane/CH₂Cl₂.

### Conditions T:

A mixture of the appropriate chloro-substrate (1 equiv), potassium carbonate (2.0 equiv), the appropriate pinacolate boron ester or boronic acid (2.0 equiv) and tetrakis(triphenylphosphine) palladium⁰ (0.05 equiv) was dissolved in acetonitrile/water (0.02 M of chloro-substrate). The reaction vessel was sealed and exposed to microwave radiation (130° C., medium absorption setting) for 30 minutes. Upon completion the sample was concentrated in vacuo. The crude residue was purified by column chromatography on silica gel eluting with 0 to 5% MeOH in CH₂Cl₂ to give the desired product.

### Conditions U:

A mixture of the appropriate chloro-substrate (1 equiv), potassium carbonate (3.0 equiv), the appropriate pinacolate boron ester or boronic acid (1.0 equiv) and tetrakis(triphenylphosphine) palladium⁰ (0.05 equiv) in acetonitrile/water (0.1 M of chloro-substrate) was stirred at 110° C. for 8 hours. Upon completion the reaction mixture was partitioned between water and CH₂Cl₂ and extracted with CH₂Cl₂. Combined organic phases were washed with brine, dried (MgSO₄), filtered and concentrated in vacuo. The crude residue was purified by column chromatography on silica gel eluting with 0 to 2% MeOH in CH₂Cl₂ to give the desired product which was recrystallised from hexane/CH₂Cl₂.

### Conditions V:

A mixture of the appropriate chloro-substrate (1 equiv), cesium fluoride (3.0 equiv), the appropriate pinacolate boron ester or boronic acid (1 equiv) and tetrakis(triphenylphosphine) palladium⁰ (0.05 equiv) in acetonitrile/water (0.1 M of chloro-substrate) was stirred at 100° C. for 16 hours. The reaction mixture was partitioned between water and CH₂Cl₂ and extracted with CH₂Cl₂. The organic phase was dried (MgSO₄), filtered and concentrated in vacuo. The crude residue was purified by column chromatography on silica gel eluting with 0 to 5% MeOH in CH₂Cl₂ to give the desired product which was recrystallised from hexane/CH₂Cl₂.

### Conditions W:

A mixture of the appropriate chloro-substrate (1 equiv), potassium carbonate (2.5 equiv), the appropriate pinacolate boron ester or boronic acid (1 equiv) and tetrakis(triphenylphosphine) palladium⁰ (0.05 equiv) was dissolved in acetonitrile/water (0.04 M of chloro-substrate). The reaction vessel was sealed and exposed to microwave radiation (110° C., medium absorption setting) for 10 minutes. The crude residue was purified by column chromatography on silica gel eluting with 0 to 2% MeOH in TBME to give the desired product.

TABLE 1

| | | Retention | | | |
|---|---|---|---|---|---|
| | Purity | time | m/z | | |
| | (%) | (min) | [M + H]⁺ | Conditions | Example Structure |
| la | 96 | 7.66 | 466.6 | A | 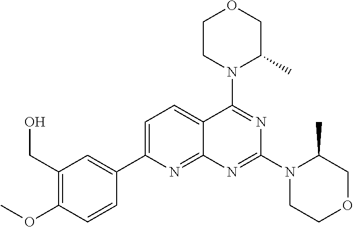 |

### Example 2

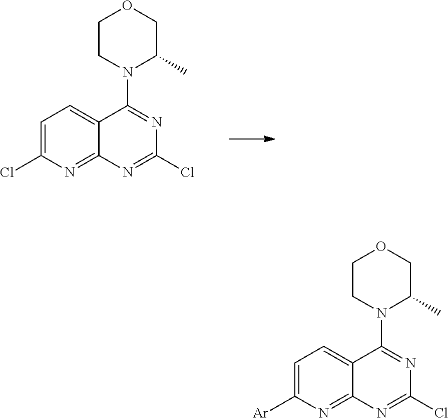

The chloro-substrate was reported in Example 1.

To a mixture of the appropriate chloro-substrate (1 equiv), potassium carbonate (2.5 equiv), and the appropriate boronic acid (1.1 equiv) in acetonitrile/water (1:1) (0.033 M of chloro-substrate) was added tetrakis(triphenylphosphine) palladium⁰ (0.05 equiv). The suspension was sonicated while degassed with nitrogen for 5 minutes then heated to 95° C. for 2 hours. Upon completion the reaction mixture was allowed to cool down to room temperature. The reaction mixture was concentrated in vacuo to half original volume. The crude residue was extracted with CH₂Cl₂ and the combined organic phases were washed with brine, dried (MgSO₄), filtered and concentrated in vacuo to give a yellow solid. The residue was sonicated in diethyl ether, collected by vacuum filtration to give the desired product as a yellow powder.

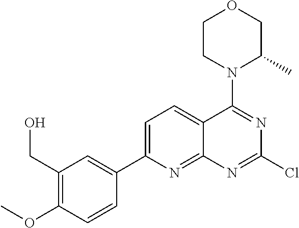

{5-[2-Chloro-4-((S)-3-methyl-morpholin-4-yl)-pyrido[2,3-d]pyrimidin-7-yl]-2-methoxy-phenyl}-methanol: (78% yield, 100% purity) m/z (LC-MS, ESP): 401 [M+H]⁺ R/T=3.47 min

Alternatively, to a stirred mixture of bis(pinacolato)diboron (1.05 equiv) and potassium acetate (3 equiv) in N-methylpyrrolidine (13.5 equiv), purged with nitrogen, was added the corresponding bromobenzylalcohol (1 equiv) followed by PdCl₂ (dppf) (0.02 equiv). The mixture was then heated to 60° C. and held for 10 min, then heated to 70° C. and held for 15 min and finally heated to 80° C. and held for 1 h. The appropriate chloro-substrate (1 equiv) was then added followed by PdCl₂ (dppf) (0.02 equiv) and N-methylpyrrolidine (4.5 equiv). The temperature was then held at 75° C., then 4.3M aqueous potassium carbonate (3.5 equiv) was added over 13 min, then water (12 equiv) was added and the reaction was stirred at 75° C. for 90 min. Water (144 equiv) was then added slowly over 70 min with stirring while the temperature was reduced to 66° C. The temperature of the stirred mixture was then kept at 64° C. for 30 min, then cooled to 20° C. over 2.5 h, and held at 20° C. overnight. The resulting slurry was filtered, and the solid washed first with a 3:1 water:N-methylpyrrolidone mixture (18 equiv of water), then washed with water (24 equiv) and then washed with ethyl actetate (4×4.4 equiv). The solid was then dried in a vacuum oven at 50° C. to leave the title compound in suitable clean form to be used without any further purification. For example, {5-[2-Chloro-4-((S)-3-methyl-morpholin-4-yl)-pyrido[2,3-d]pyrimidin-7-yl]-2-methoxy-phenyl}-methanol: (73% yield)

### (Compounds 2a to 2b)

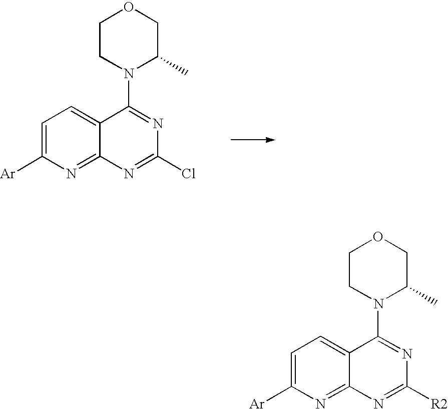

### Conditions A:

The appropriate chloro-substrate (1 equiv) was dissolved in DMA (0.04 M). Tripotassium phosphate (1.5 equiv) and the appropriate nucleophile (secondary amine) (1.5 equiv) were then added. The reaction vessel was sealed and the mixture exposed to microwave radiation (200° C., medium absorption setting) for 30 minutes. Upon completion the samples were filtered through a silica cartridge, washed with EtOAc and then concentrated in vacuo. The crude residue was then purified by preparative HPLC to give the desired products.

### Conditions B:

The appropriate chloro-substrate (1 equiv) was suspended in a propan-2-ol and aqueous ammonia (1:3) solution (0.02 M). The reaction vessel was sealed and the mixture exposed to microwave radiation (140° C., medium absorption setting) for 20 minutes. The crude residue was then purified by preparative HPLC to give the desired products.

### Conditions C:

The appropriate chloro-substrate (1 equiv) was dissolved in dioxane (0.04 M). Diisopropylethylamine (5.0 equiv) and the appropriate nucleophile (secondary amine) (1.5 equiv) were then added. The reaction vessel was sealed and the mixture exposed to microwave radiation (130° C., medium absorption setting) for 20 minutes. Upon completion the samples were concentrated in vacuo. The crude residue was then purified by preparative HPLC to give the desired products.

### Conditions D:

The appropriate chloro-substrate (1 equiv) was dissolved in dioxane (0.04 M). Tripotassium phosphate (3.0 equiv), xantphos (0.05 equiv), palladium acetate (0.05 equiv) and the appropriate nucleophile (amine) (1.5 equiv) were then added. The reaction vessel was sealed and the mixture exposed to microwave radiation (150° C., medium absorption setting) for 20 minutes. Upon completion the samples were filtered through a silica cartridge, washed with EtOAc and then concentrated in vacuo. The crude residue was then purified by preparative HPLC to give the desired products.

### Conditions E:

The appropriate chloro-substrate (1.0 equiv) was dissolved in dioxane (0.04 M). Diisopropylethylamine (5.0 equiv) and the appropriate nucleophile (secondary amine, with BOC-protected amino side chain) (1.5 equiv) were then added. The reaction vessel was sealed and the mixture exposed to microwave radiation (130° C., medium absorption setting) for 20 minutes. Upon completion the samples were concentrated in vacuo. To the crude residue was then added a 4 M solution of HCl in dioxane (0.15 M). The reaction mixtures were stirred at room temperature for 3 hours. Upon completion the samples were basified with a 2 N sodium hydroxide solution. The crude residue was then purified by preparative HPLC to give the desired products.

### Conditions F:

The appropriate nucleophile (substituted imidazole) (10.0 equiv) was dissolved in DMF (0.4 M). Sodium hydride (5.0 equiv) was then added. The reaction mixture was stirred at room temperature for 10 minutes under nitrogen and a solution of the appropriate chloro-substrate (1.0 equiv) in DMF (0.075 M) was added. The reaction vessel was sealed and the mixture exposed to microwave radiation (150° C., medium absorption setting) for 30 minutes. Upon completion the samples were filtered through a silica cartridge, eluted with CH₂Cl₂ and then concentrated in vacuo. The crude residue were then purified by preparative HPLC to give the desired products.

### Conditions G:

The appropriate chloro-substrate (1 equiv) was dissolved in dioxane (0.04 M). Diisopropylethylamine (5.0 equiv) and the appropriate nucleophile (secondary amine) (4.5 equiv) were then added. The reaction vessel was sealed and the mixture exposed to microwave radiation (130° C., medium absorption setting) for 40 minutes. Upon completion the samples were concentrated in vacuo. The crude residue was then purified by preparative HPLC to give the desired products.

### Conditions H:

The appropriate chloro-substrate (1 equiv) was dissolved in dioxane (0.04 M). Diisopropylethylamine (5.0 equiv) and the appropriate nucleophile (secondary amine) (10.0 equiv) were then added. The reaction vessel was sealed and the mixture exposed to microwave radiation (130° C., medium absorption setting) for 60 minutes. Upon completion the samples were concentrated in vacuo. The crude residue was then purified by preparative HPLC to give the desired products.

### Conditions I:

The appropriate chloro-substrate (1 equiv) was dissolved in a solution of 1% DMA in dioxane (0.04 M). Diisopropylethylamine (5.0 equiv) and the appropriate nucleophile (secondary amine) (10.0 equiv) were then added. The reaction vessel was sealed and the mixture exposed to microwave radiation (180° C., medium absorption setting) for 60 minutes. Upon completion the samples were concentrated in vacuo. The crude residue was then purified by preparative HPLC to give the desired products.

### Conditions J:

The appropriate chloro-substrate (1 equiv) was dissolved in a solution of 1% DMA in dioxane (0.04 M). Diisopropylethylamine (7.0 equiv) and the appropriate nucleophile (secondary amine) (3.0 equiv) were then added. The reaction vessel was sealed and the mixture exposed to microwave radiation (150° C., medium absorption setting) for 60 minutes. Upon completion the samples were concentrated in vacuo. The crude residue was then purified by preparative HPLC to give the desired products.

### Conditions K:

The appropriate chloro-substrate (1 equiv) was dissolved in DMF (0.075 M). Potassium carbonate (5.0 equiv) and the appropriate nucleophile (alcohol) (10.0 equiv) were then added. The reaction vessel was sealed and the mixture exposed to microwave radiation (120° C., medium absorption setting) for 20 minutes. Upon completion the samples were concentrated in vacuo. The crude residue was then purified by preparative HPLC to give the desired products.

### Conditions L:

The appropriate chloro-substrate (1 equiv) was dissolved in DMF (0.075 M). Potassium carbonate (5.0 equiv) and the appropriate nucleophile (alcohol) (20.0 equiv) were then added. The reaction vessel was sealed and the mixture exposed to microwave radiation (150° C., medium absorption setting) for 40 minutes. Upon completion the samples were concentrated in vacuo. The crude residue was then purified by preparative HPLC to give the desired products.

### Conditions M:

The appropriate chloro-substrate (1 equiv) was dissolved in DMA (0.13 M). Diisopropylethylamine (2.0 equiv) and the appropriate nucleophile (amine) (2.0 equiv) were then added. The reaction vessel was heated to 100° C. for 3 hours. Upon completion, the reaction mixture was partitioned between dichloromethane and water and the aqueous layer further extracted with dichloromethane. The combined organic phases were dried (MgSO₄), filtered and the filtrate was concentrated in vacuo to give a yellow residue which was purified by recrystallisation from diethyl ether.

### Conditions N:

5-[2-Chloro-4-((S)-3-methyl-morpholin-4-yl)-pyrido[2,3-d]pyrimidin-7-yl]-pyridin-2-ylamine (1 equiv) was dissolved in DMA (0.21 M). Diisopropylethylamine (1.0 equiv) and the appropriate nucleophile (amine) (1.1 equiv) were then added. The reaction vessel was sealed and the mixture exposed to microwave radiation (130° C., medium absorption setting) for 10 minutes. Upon completion, the reaction mixture was partitioned between dichloromethane and water and the aqueous layer further extracted with CH₂Cl₂. The combined organic phases were dried (MgSO₄), filtered and the filtrate was concentrated in vacuo to give a yellow residue which was purified by column chromatography on silica gel eluting with 0% to 10% MeOH in CH₂Cl₂ to give the desired product.

### Conditions O:

The appropriate chloro-substrate (1 equiv) was dissolved in DMA (0.16 M). Diisopropylethylamine (1.0 equiv) and the appropriate nucleophile (amine) (1.2 equiv) were then added. The reaction vessel was heated to 80° C. for 48 hours. Upon completion, the reaction mixture was partitioned between ethyl acetate and water and the organic layer washed with brine. The combined organic phases were dried (MgSO₄), filtered and the filtrate was concentrated in vacuo to give a residue which was purified by preparative HPLC to give the desired product.

### Conditions P:

The appropriate chloro-substrate (1 equiv) was dissolved in anisole (0.25 M) (10 vol). Diisopropylethylamine (1.3 equiv) and the appropriate nucleophile (amine) (1.3 equiv) were then added. The reaction vessel was heated to 125° C. and stirred for 11 h. Upon completion, the reaction mixture was allowed to cool to 50° C. Aqueous 20% citric acid solution (7 vol) was added, stirred for 5 min and then allowed to separate partitioned. The aqueous layer was removed and retained. The organic layer was then extracted with a further aliquot of aqueous 20% citric acid solution (3 vol). The organic layer discarded, and the aqueous layers combined. The combined aqueous layers were washed first with anisole (5 vol), then 50% aqueous sodium hydroxide solution (1.23 vol) was added slowly. The resulting aqueous phase was extracted with ethyl acetate (10 vol). The aqueous layer was discarded and the organic layer was washed first with 10% aqueous sodium hydroxide solution (5 vol) and then water (5 vol). The organic layer was then slurried with silicycle Si-thiourea scavenger at 50° C. for 2 h, then the scavenger was filtered off and washed with ethyl actetate (2×1 vol). The organic phase was cooled to 20° C., seeded to start crystallization and stirred until a slurry obtained. The slurry was heated to 50° C. under vacuum and ethyl acetate (3 vol) was removed by vacuum distillation. 2-Methylpentane (3.4 vol) was added and the mixture heated to 60° C. and then slowly cooled to 20° C. over 2 h. The resulting slurry was filtered, and the solid washed with 1:1 ethyl actetate:pentane (2×0.5 vol). The solid was then dried in a vacuum oven at 50° C. to leave the desired product. For example, compound 1a was obtained (50.4% yield). The crude product (1 equiv) was dissolved in DMSO (5 vol based on product weight) at 50° C. Water (2 vol) was added and the mixture stirred at 50° C. until product crystallizes. The slurry was heated to 60° C. and then water (3 vol) was added slowly over 30 min so that the temperature was maintained at 60° C. The mixture was slowly cooled to 20° C. over 2 h, and then held at 20° C. for 30 min. The resulting slurry was filtered, and the solid washed with 2:1 water:DMSO (0.5:1 vol), and then water (3×2 vol). The solid was then dried in a vacuum oven at 50° C. to leave the desired product.

TABLE 2

| | | Retention | | | |
|---|---|---|---|---|---|
| | Purity | time | m/z | | |
| | (%) | (min) | [M + H]⁺ | Conditions | Example Structure |
| 2a | 97 | 4.03 | 466.2 | O | 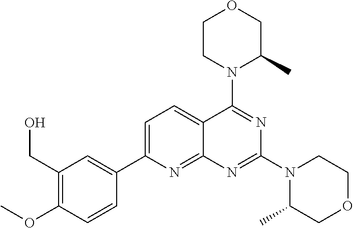 |
| 2b | 99 | 3.99 | 466.2 | O | 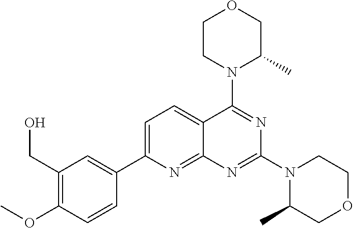 |

### NMR Data for Example 2a

¹H NMR (300 MHz, CDCl₃ δ ppm 8.10 (ArH, d, J=7.89 Hz, 2H), 7.97 (ArH, d, J=8.49 Hz, 1H), 7.42 (ArH, d, J=8.46 Hz, 1H), 6.98 (ArH, d, J=8.55 Hz, 1H), 4.88 (CH₂, d, J=5.25 Hz, 1H), 4.77 (CH₂OH, s, 2H), 4.56 (CH₂, d, J=13.38 Hz, 1H), 4.38-4.36 (CH₂, m, 1H), 4.02-3.51 (OCH₃+CH₂, m, 11H), 3.43-3.33 (CH₂, m, 1H), 1.47 (CH₃, d, J=6.77 Hz, 3H), 1.35 (CH₃, d, J=6.78 Hz, 3H).

¹³C NMR (75 MHz, CD₃COCD₃) δ ppm 165.11, 162.27, 161.87, 159.54, 159.23, 134.74, 130.76, 129.41, 128.86, 128.39, 113.09, 110.32, 104.45, 71.20, 70.95, 67.17, 66.91, 61.80, 55.57, 52.82, 47.05, 44.44, 39.45, 14.74 and 14.44.

### NMR Data for Example 2b

¹H NMR (300 MHz, CDCl₃) δ ppm 8.10 (ArH, d, J=8.76 Hz, 2H), 7.98 (ArH, d, J=8.49 Hz, 1H), 7.42 (ArH, d, J=8.46 Hz, 1H), 6.97 (ArH, d, J=8.37 Hz, 1H), 4.88 (CH₂, d, J=5.46 Hz, 1H), 4.77 (CH₂OH, s, 2H), 4.58-4.49 (CH₂, m, 1H), 4.39-4.36 (CH₂, d J=7.41 Hz, 1H), 4.02-3.51 (OCH₃+CH₂, m, 11H), 3.43-3.33 (CH₂, m, 1H), 1.48 (CH₃, d, J=6.78 Hz, 3H), 1.35 (CH₃, d, J=6.78 Hz, 3H).

¹³C NMR (75 MHz, CD₃COCD₃) δ ppm 165.05, 161.87, 159.45, 159.24, 134.78, 130.70, 129.44, 128.86, 128.38, 113.14, 110.33, 104.43, 71.19, 70.95, 67.16, 66.90, 61.77, 55.57, 52.82, 47.08, 44.44, 39.47, 14.76 and 14.44.

### Example 3

### In Vivo Combination Study of AZD2171 with the mTOR-Selective Kinase Inhibitor [5-[2,4-bis[(3S)-3-methylmorpholin-4-yl]pyrido[5,6-e]pyrimidin-7-yl]-2-methoxyphenyl]methanol (“Compound A”), in the NCI-H256 Tumour Model

Cells were grown in NCI-H526 (RPMI 10% FCS 1% Glutamine 10% Ml), and 1×10⁷ cells were implanted into the flank of nude mice (NCI-H526 plus 50% matrigel).

When mean tumor size reached approx 0.2 cm³, the mice were randomized into control and treatment groups. The treatment groups received either 3 mg/kg AZD2171 (vehicle 1% polysorbate 80 milled overnight), or 20 mg/kg COMPOUND A (vehicle 10% DMSO, 90% propylene glycol) by oral gavage. When administered in combination, COMPOUND A was given 2 hours after the oral dose of the other compound. The control group received the (10% DMSO 90% propylene glycol) vehicle alone, once daily by oral gavage. Tumor volumes (measured by calliper), animal body weight and tumor condition were recorded twice weekly for the duration of the study. Mice were sacrificed by CO₂ euthanasia. The tumor volume was calculated (taking length to be the longest diameter across the tumor and width to be the corresponding perpendicular diameter using the formula: (length×width)×√(length×width)×(π/6). Growth inhibition from the start of treatment was assessed by comparison of the differences in tumor volume between control and treated groups. Because the variance in mean tumor volume data increases proportionally with volume (and is therefore disproportionate between groups), data were log-transformed to remove any size dependency before statistical evaluation. Statistical significance was evaluated using a one-tailed, two-sample t test. To analyze the data from the combination study, the statistical tool SigmaStat has been used. A two-way ANOVA test was performed using the factors concentration of drug A and concentration of drug B. The data analyzed was Log(final tumor volume)−Log(initial tumor volume) calculated for each individual group at the end of the study. This tool is used to assess whether there is a main effect of drug A, a main effect of drug B plus a significant mechanistic interaction between the two compounds A and B (eg, one compound influences the effect of the other compound) which may be interpreted as antagonism, additivity or synergism.

In the NCI-H526 model, 20 mg/kg of COMPOUND A gave a 38% reduction in geometric mean delta tumour volume (p=0.02 compared with the vehicle control), 3 mg/kg AZD2171 gave a 64% reduction in geometric mean delta tumour volume (p=0.005) and the combination of the same doses of these two agents resulted in an 84% reduction in geometric mean delta tumour volume (p<0.0001). The administration of the combination was significantly more effective than the administration of the monotherapy at inhibiting tumour growth (combination versus COMPOUND A monotherapy 59% effect, p<0.0001, combination versus AZD2171 monotherapy 37% effect, p=0.01). The data is shown graphically in FIG. 1.

AZD2171 (3 mg/kg)+Compound A (20 mg/kg) vs. AZD2171 (3 mg/kg): p=0.01 (2-tailed t-test)
AZD2171 (3 mg/kg)+Compound A (20 mg/kg) vs. Compound A (3 mg/kg): p<0.0001 (2-tailed t-test).
There was no significant difference in body weight loss between the 3 groups i.e combination of compound A and compound B did not decrease the tolerance of the therapy.

### Example 4

### In Vivo Combination Study of AZD2171 with the mTOR-Selective Kinase Inhibitor [5-[2,4-bis[(3S)-3-methylmorpholin-4-yl]pyrido[5,6-e]pyrimidin-7-yl]-2-methoxyphenyl]methanol (“Compound A”), in the A549 Tumour Model

Human tumour xenografts were established in female athymic mice by injecting 5×10⁶/0.1 ml A549a cells subcutaneously in the left hand side flank.

When mean tumour volume reached approximately 0.27 cm³, the mice were randomized into control and treatment groups. The treatment groups received 3 mg/kg AZD2171 (vehicle 1% polysorbate 80 milled overnight) or 20 mg/kg COMPOUND A (30% Captisol pH5) or a combination 3 mg/kg AZD2171 and 20 mg/kg COMPOUND A by oral gavage. When administered in combination, COMPOUND A was given 2 hours before the oral dose of AZD2171. The control group received both vehicles alone (same sequence as above), once daily by oral gavage. Tumor volumes (measured by calliper), animal body weight and tumor condition were recorded twice weekly for the duration of the study. Mice were humanely sacrificed by CO₂ euthanasia. Tumour volumes were assessed using bilateral Vernier calliper measurement at least twice weekly and calculated using the formula (length×width)×√(length×width)×(π/6), where length was taken to be the longest diameter across the tumour and width the corresponding perpendicular. Growth inhibition from the start of treatment was assessed by comparison of the differences in tumor volume between control and treated groups. Because the variance in mean tumor volume data increases proportionally with volume (and is therefore disproportionate between groups), data were log-transformed to remove any size dependency before statistical evaluation. Statistical significance was evaluated using a one-tailed, two-sample t test. To analyze the data from the combination study, the statistical tool SigmaStat has been used. A two-way ANOVA test was performed using the factors concentration of drug A and concentration of drug B. The data analyzed was Log(final tumor volume)−Log(initial tumor volume) calculated for each individual animal at the end of the study. This tool is used to assess whether there is a main effect of drug A, a main effect of drug B plus a significant mechanistic interaction between the two compounds A and B (eg, one compound influences the effect of the other compound) which may be interpreted as antagonism, additivity or synergism. The statistical analysis was performed at the end of the dosing period (21 days).

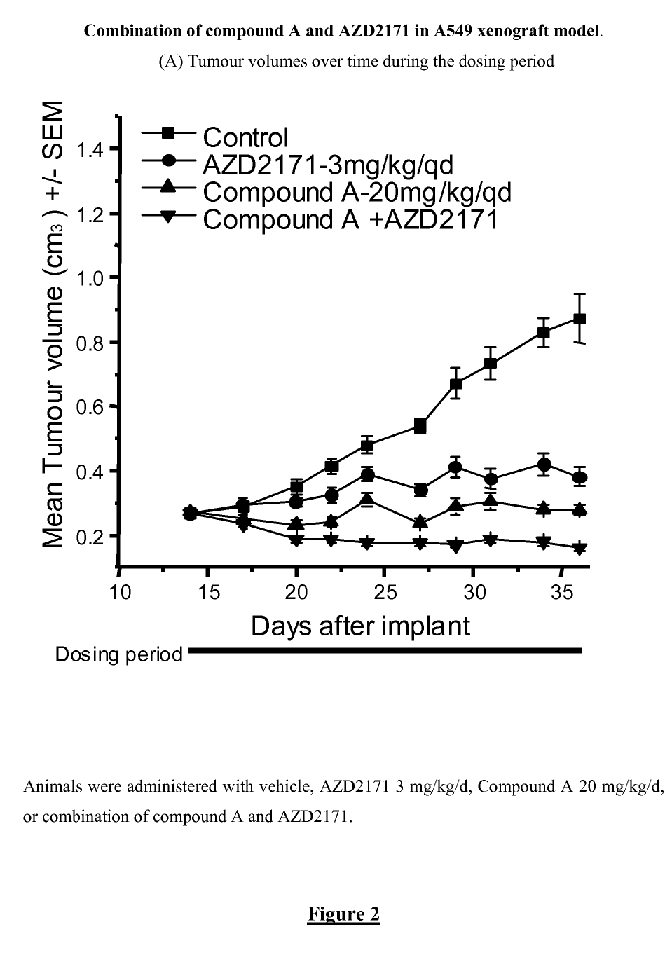

In the A549 model, 20 mg/kg of COMPOUND A inhibited tumour growth tumour by 99.3% (p<0.0001 compared to control), 3 mg/kg/qd induced 81.6% tumour growth inhibition (p<0.0001 compared to control) and Compound A in combination with AZD2171 inhibited tumour growth by 119.1% (p<0.0001 compared to control). There was a statistically significant interaction between the 2 compounds (p=0.05). The data is shown graphically in FIG. 2. When considering individual tumour volumes at the end of the dosing period, all the tumours in the control and AZD2171-treated groups, tumour volumes were greater than at start of the treatment. After Compound A, 4 out of 10 tumours were smaller than at start of treatment. In the combination group, 10 out of 10 xenografts were smaller than at start of treatment. The maximum body weight loss were 4.43% (0.8-15.4), 2.52% (0.8-4.7), compound A 3.45% (0-17.1), and 4.63% (0-8) for controls, AZD2171, Compound A and the combination, respectively.

A pharmacodynamic study evaluated biomarkers after 4 days of treatment. Animals were dosed with vehicle, AZD2171 (3 mg/kg), Compound A (20 mg/kg) or the combination daily. Compound A was dosed two hours before AZD2171 (or vehicle). Animals were culled and tumour xenograft tissue collected 2 hrs after the last dosing. To analyze the data from the pharmacodynamic study, a t-test was performed to compare the effect of each treatment groups to the effect observed in the control group. In addition a two-way ANOVA test was performed to analyse the interaction between compounds in the combination group.

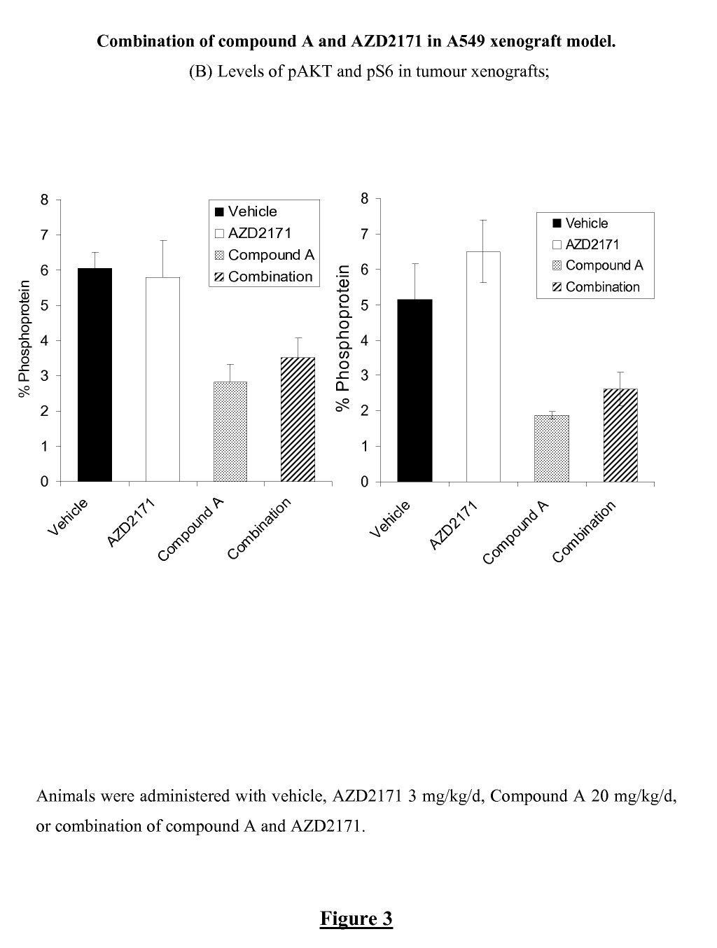

Levels of pAKT (normalised to total) were decreased to 48% (p<0.005) compared to control levels with Compound A and to 95% (p=0.41) after AZD2171. In the combination group, levels of pAKT were similar to those with Compound A to 58% (p<0.005) compared to control (FIG. 3). There was no statistical interaction between compound A and AZD2171 in the combination-treated tumours (p=0.46).

Levels of pS6 (normalised to total) were decreased to 37% (p<0.05) compared to control levels with Compound A and slightly increased to 126% after AZD2171 (p=0.17). In the combination group, levels of pS6 decreased to 51% (p<0.05) compared to control (FIG. 3). There was no statistical interaction between compound A and AZD2171 in the combination-treated tumours (p=0.67).

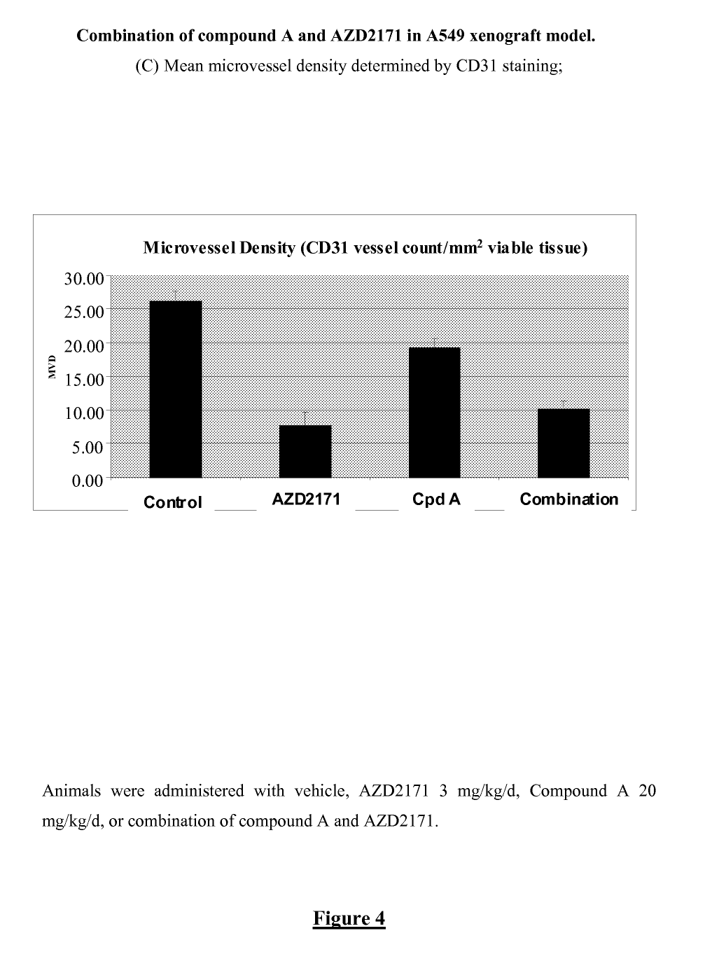

Microvessel density (MVD) was evaluated using CD31 staining by immunohistochemistry 2 hours after 4 days of treatment. MVD values were 26.6% in vehicle-treated animals, reduced to 7.75% (p<0.0005) after AZD2171, 19.39% (p<0.005) after Compound A, and 10.24% (p<0.0005) after the combination (FIG. 4). Both compound A and AZD2171 are contributing to the effect seen in the combination-treated tumours (p=0.005).

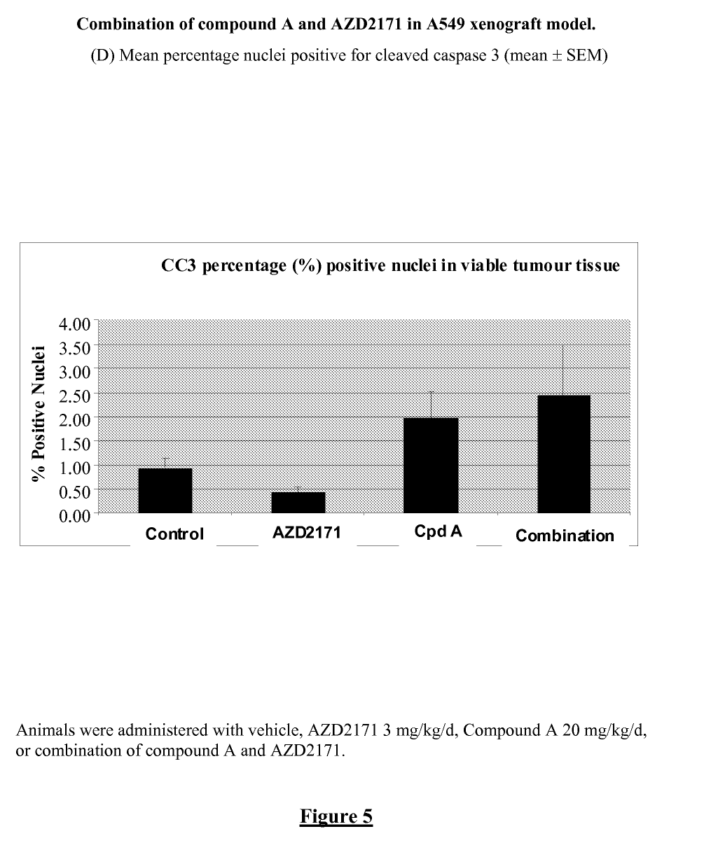

The levels of cleaved caspase 3 (percentage of positive nuclei) were determined in tumour xenografts by immunohistochemistry 2 hours after 4 days of treatment. CC3 values were 0.94% in the control group, 0.43% (p<0.05) after AZD2171, 1.98% (p=0.06) after compound A and 2.43% (p=0.12) after the combination (FIG. 5). There was no statistical interaction between compound A and AZD2171 in the combination-treated tumours (p=0.44).

### Example 5

### In Vivo Combination Study of ZD6474 with the mTOR-Selective Kinase Inhibitor [5-[2,4-bis[(3S)-3-methylmorpholin-4-yl]pyrido[5,6-e]pyrimidin-7-yl]-2-methoxyphenyl]methanol (“Compound A”), in the U87-MG Tumour Model

Human tumour xenografts were established in female athymic mice by injecting 2.3×10⁶/0.1 ml U87MG cells subcutaneously in the left hand side flank. When mean tumour volume reached approximately 0.18 cm³, the mice were randomized into control and treatment groups. The treatment groups received 50 mg/kg ZD6474 (vehicle 1% polysorbate 80 milled overnight) or 10 mg/kg COMPOUND A (30% captisol pH5) or a combination 50 mg/kg ZD6474 and 10 mg/kg COMPOUND A by oral gavage. When administered in combination, COMPOUND A was given 2 hours before the oral dose of ZD6474. The control group received both vehicles (same sequence as above), once daily by oral gavage. Tumor volumes (measured by calliper), animal body weight and tumor condition were recorded twice weekly for the duration of the study. Mice were humanely sacrificed by CO₂ euthanasia. Tumour volumes were assessed using bilateral Vernier calliper measurement at least twice weekly and calculated using the formula (length×width)×√(length×width)×(π/6), where length was taken to be the longest diameter across the tumour and width the corresponding perpendicular. Growth inhibition from the start of treatment was assessed by comparison of the differences in tumor volume between control and treated groups. Because the variance in mean tumor volume data increases proportionally with volume (and is therefore disproportionate between groups), data were log-transformed to remove any size dependency before statistical evaluation. Statistical significance was evaluated using a one-tailed, two-sample t test. To analyze the data from the combination study, the statistical tool SigmaStat has been used. A two-way ANOVA test was performed using the factors concentration of drug A and concentration of drug B. The data analyzed was Log(final tumor volume)−Log(initial tumor volume) calculated for each individual animal at the end of the study. This tool is used to assess whether there is a main effect of drug A, a main effect of drug B plus a significant mechanistic interaction between the two compounds A and B (eg, one compound influences the effect of the other compound) which may be interpreted as antagonism, additivity or synergism. The statistical analysis was performed at the end of the dosing period (7 days).

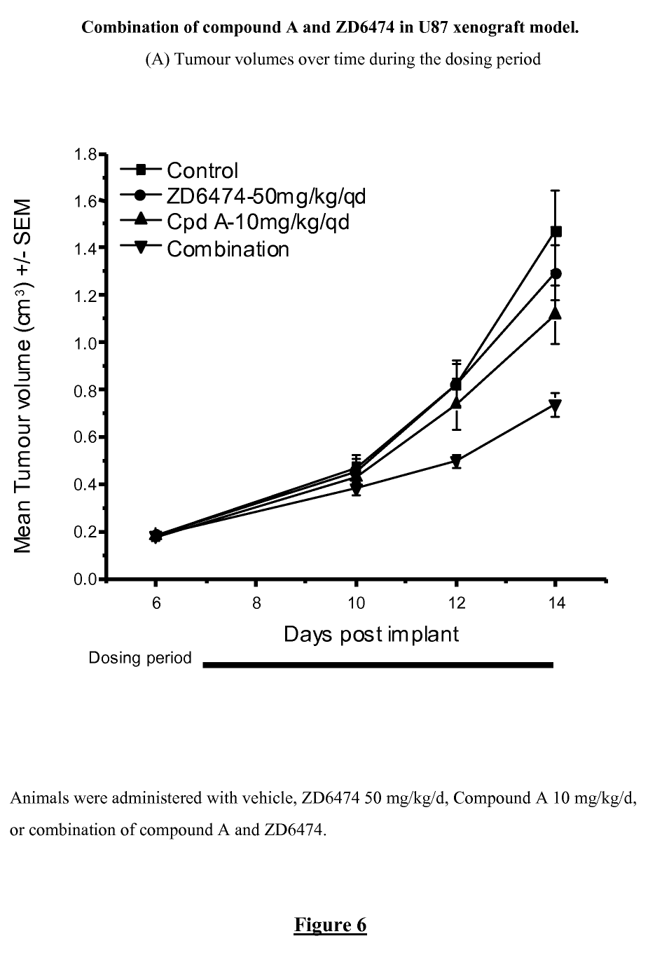

Compound A at 10 mg/kg/qd inhibited U87MG tumour growth by 28.4% (p<0.0001 compared to control). ZD6474 at 50 mg/kg/qd did not significantly inhibit the growth of U87MG xenografts (−14.6%; NS compared to control). Compound A in combination with ZD6474 inhibited tumour growth in U87MG xenografts by 63.6% (p=0.0004 compared to control). There was no statistically significant interaction between the 2 compounds (p=0.15). The data is shown graphically in FIG. 6. The maximum body weight loss were 0.71% (0-2.5), 3.77% (0-11.2), 0.44% (0-2.8), and 3.75% (0-11.1) for control, ZD6474, Compound A and the combination, respectively.

A pharmacodynamic study evaluated biomarkers after 4 days of treatment. Animals were dosed with vehicle, ZD6474 (50 mg/kg), Compound A (10 mg/kg) or the combination daily. Compound A was dosed two hours before ZD6474 (or vehicle). Animals were culled and tumour xenograft tissue collected 2 hrs after the last dosing. To analyze the data from the pharmacodynamic study, a t-test was performed to compare the effect of each treatment groups to the effect observed in the control group. In addition a two-way ANOVA test was performed to analyse the interaction between compounds in the combination group.

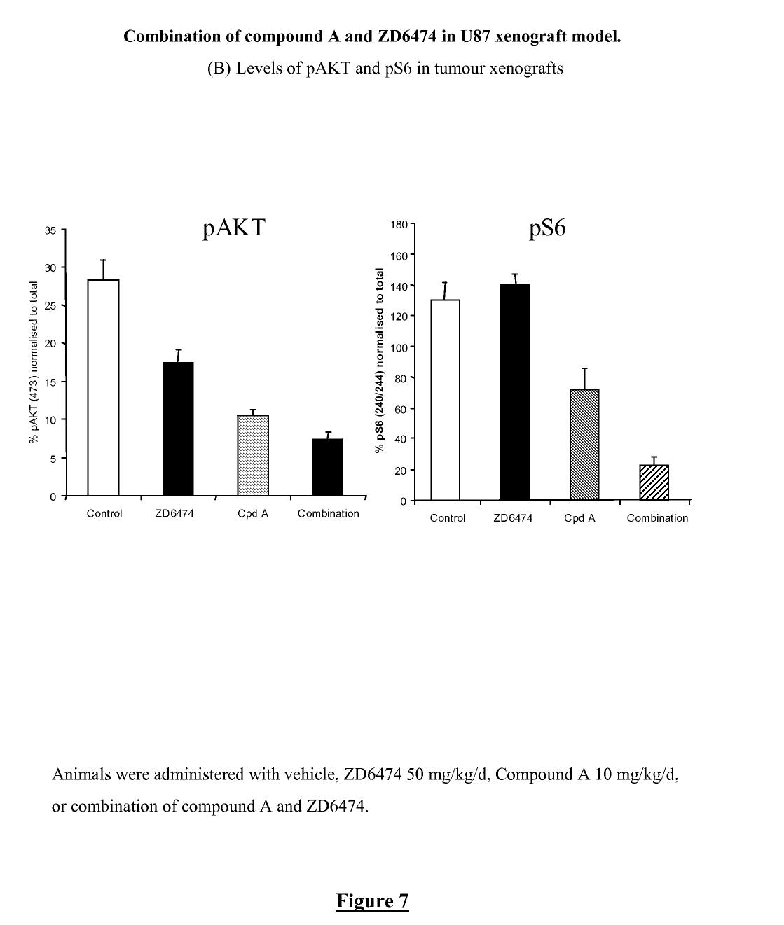

Levels of pAKT (normalised to total) were decreased to 37% (p<0.005) compared to control levels with Compound A and to 87% after ZD6474 (p<0.05). In the combination group, levels of pAKT were similar to those with Compound A to 26% (p<0.0005) compared to control (FIG. 7). Both compound A and ZD6474 are contributing to the effect seen in the combination-treated tumours (p<0.05).

Levels of pS6 (normalised to total) were decreased to 55% (p<0.05) compared to control levels with Compound A and similar to control levels after ZD6474 (108%; p=0.24)). In the combination group, levels of pS6 decreased to 18% (p<0.0005) compared to control (FIG. 7). Both compound A and ZD6474 are contributing to the effect seen in the combination-treated tumours (p<0.05).

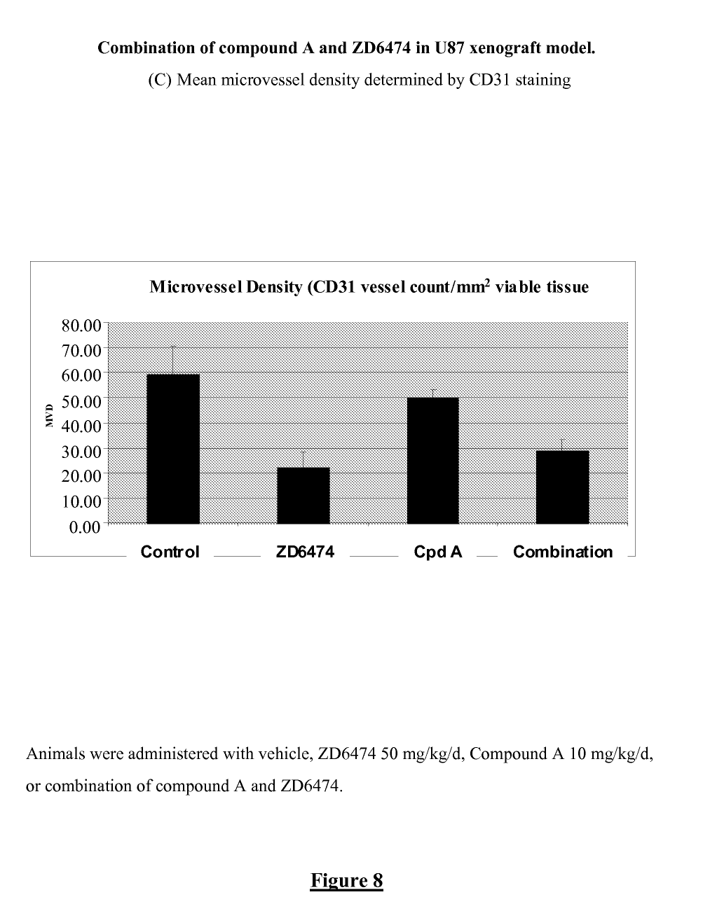

Microvessel density (MVD) was evaluated using CD31 staining by immuno-histochemistry 2 hours after 4 days of treatment. MVD values were 59% in vehicle-treated animals, reduced to 22% (p<0.05) after ZD6474, 50% (p=0.23) after Compound A, and 28% (p<0.05) after the combination (FIG. 8). There was no statistical interaction between compound A and ZD6474 in the combination-treated tumours (p=0.27).

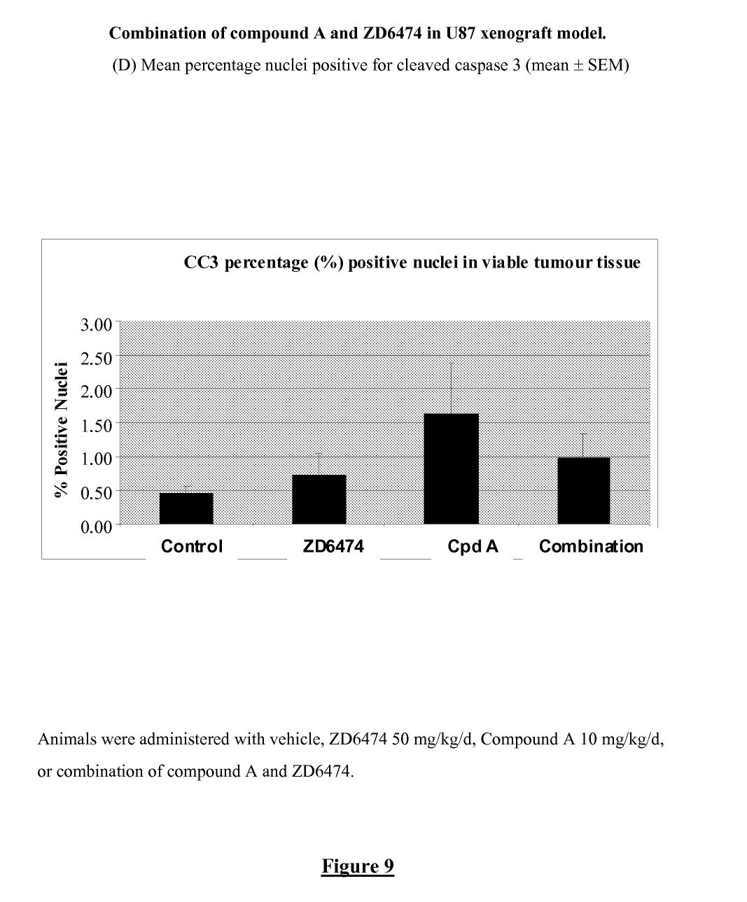

The levels of cleaved caspase 3 (percentage of positive nuclei) were determined in tumour xenografts by immunohistochemistry 2 hours after 4 days of treatment. CC3 values were 0.46% in the control group, 0.73% (p=0.23) after ZD6474, 1.64% (p=0.09) after compound A and 0.99% (p=0.09) after the combination (FIG. 9). There was no statistical interaction between compound A and ZD6474 in the combination-treated tumours (p=0.31).

### CONCLUSION

The combination of compound A with VEGFR inhibitor AZD2171 or ZD6474 increases the antitumour activity of agents used as monotherapy.
In the combination AZD2171-Compound A, the tumour volumes decreased below their initial volumes in 10 out of 10 animals in the combination compared to 4 out of 10 animals with Compound A alone, suggesting tumour regression.
The combination does not affect the tolerability of the treatments as measured by body weight loss during the course of the treatment.

The pharmacodynamic studies are consistent with the compounds exerting their respective pharmacology.

1. A combination product comprising a VEGFR tyrosine kinase inhibitor, or a pharmaceutically acceptable salt thereof, and a mTOR-selective kinase inhibitor, or a pharmaceutically acceptable salt thereof, in association with a pharmaceutically acceptable adjuvant, diluent or carrier.

2. A combination product according to claim 1 comprising 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor, or a pharmaceutically acceptable salt thereof, in association with a pharmaceutically acceptable adjuvant, diluent or carrier
wherein the mTOR-selective kinase inhibitor is selected from any one of
5-[2,4-bis[3-methylmorpholin-4-yl]pyrido[5,6-e]pyrimidin-7-yl]-2-methoxy-N-methylbenzamide;
4-[2,4-bis[3-methylmorpholin-4-yl]pyrido[5,6-e]pyrimidin-7-yl]aniline;
6-[2,4-bis[3-methylmorpholin-4-yl]pyrido[5,6-e]pyrimidin-7-yl]-1H-indazol-3-amine;
8-[2,4-bis[3-methylmorpholin-4-yl]pyrido[5,6-e]pyrimidin-7-yl]-1,2,3,4-tetrahydro-1,4-benzodiazepin-5-one;
5-[2,4-bis[3-methylmorpholin-4-yl]pyrido[5,6-e]pyrimidin-7-yl]-2-methoxy-N-methylbenzamide;
5-[2,4-bis[3-methylmorpholin-4-yl]pyrido[5,6-e]pyrimidin-7-yl]pyridin-2-amine;
N-[3-[2,4-bis[3-methylmorpholin-4-yl]pyrido[5,6-e]pyrimidin-7-yl]phenyl]methanesulfonamide;
3-[2,4-bis[3-methylmorpholin-4-yl]pyrido[5,6-e]pyrimidin-7-yl]aniline;
5-[2,4-bis[3-methylmorpholin-4-yl]pyrido[6,5-d]pyrimidin-7-yl]-2-ethoxybenzamide;
5-[2,4-bis[3-methylmorpholin-4-yl]pyrido[5,6-e]pyrimidin-7-yl]-2-(difluoromethoxy)-N-methylbenzamide;
5-[2,4-bis[3-methylmorpholin-4-yl]pyrido[5,6-e]pyrimidin-7-yl]-1H-indazol-3-amine;
[5-[2,4-bis[3-methylmorpholin-4-yl]pyrido[5,6-e]pyrimidin-7-yl]-2-methoxyphenyl]methanol;
N-[[4-[2,4-bis[3-methylmorpholin-4-yl]pyrido[5,6-e]pyrimidin-7-yl]phenyl]methyl]methanesulfonamide;
5-[2,4-bis[3-methylmorpholin-4-yl]pyrido[5,6-e]pyrimidin-7-yl]-1,3-dihydroindol-2-one;
6-[2,4-bis[3-methylmorpholin-4-yl]pyrido[5,6-e]pyrimidin-7-yl]-1,3-dihydroindol-2-one;
3-[2,4-bis[3-methylmorpholin-4-yl]pyrido[5,6-e]pyrimidin-7-yl]-N-methylbenzamide;
5-[2,4-bis[3-methylmorpholin-4-yl]pyrido[5,6-e]pyrimidin-7-yl]-2-(difluoromethoxy)benzamide;
6-[2,4-bis[3-methylmorpholin-4-yl]pyrido[5,6-e]pyrimidin-7-yl]-2,3-dihydroisoindol-1-one;
[5-[2-(2,6-dimethylmorpholin-4-yl)-4-morpholin-4-ylpyrido[6,5-d]pyrimidin-7-yl]-2-methoxyphenyl]methanol; and
[2-methoxy-5-[2-(3-methylmorpholin-4-yl)-4-morpholin-4-ylpyrido[6,5-d]pyrimidin-7-yl]phenyl]methanol.

3. A combination product according to claim 1 comprising 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor, or a pharmaceutically acceptable salt thereof, in association with a pharmaceutically acceptable adjuvant, diluent or carrier
wherein the mTOR-selective kinase inhibitor is selected from any one of
5-[2,4-bis[3-methylmorpholin-4-yl]pyrido[5,6-e]pyrimidin-7-yl]-2-methoxy-N-methylbenzamide;
4-[2,4-bis[3-methylmorpholin-4-yl]pyrido[5,6-e]pyrimidin-7-yl]aniline;
6-[2,4-bis[3-methylmorpholin-4-yl]pyrido[5,6-e]pyrimidin-7-yl]-1H-indazol-3-amine;
8-[2,4-bis[3-methylmorpholin-4-yl]pyrido[5,6-e]pyrimidin-7-yl]-1,2,3,4-tetrahydro-1,4-benzodiazepin-5-one;
5-[2,4-bis[3-methylmorpholin-4-yl]pyrido[5,6-e]pyrimidin-7-yl]-2-methoxy-N-methylbenzamide;
5-[2,4-bis[3-methylmorpholin-4-yl]pyrido[5,6-e]pyrimidin-7-yl]pyridin-2-amine;
N-[3-[2,4-bis[3-methylmorpholin-4-yl]pyrido[5,6-e]pyrimidin-7-yl]phenyl]methanesulfonamide;
3-[2,4-bis[3-methylmorpholin-4-yl]pyrido[5,6-e]pyrimidin-7-yl]aniline;
5-[2,4-bis[3-methylmorpholin-4-yl]pyrido[6,5-d]pyrimidin-7-yl]-2-ethoxybenzamide;
5-[2,4-bis[3-methylmorpholin-4-yl]pyrido[5,6-e]pyrimidin-7-yl]-2-(difluoromethoxy)-N-methylbenzamide;
5-[2,4-bis[3-methylmorpholin-4-yl]pyrido[5,6-e]pyrimidin-7-yl]-1H-indazol-3-amine;
[5-[2,4-bis[3-methylmorpholin-4-yl]pyrido[5,6-e]pyrimidin-7-yl]-2-methoxyphenyl]methanol;
N-[[4-[2,4-bis[3-methylmorpholin-4-yl]pyrido[5,6-e]pyrimidin-7-yl]phenyl]methyl]methanesulfonamide;
5-[2,4-bis[3-methylmorpholin-4-yl]pyrido[5,6-e]pyrimidin-7-yl]-1,3-dihydroindol-2-one;
6-[2,4-bis[3-methylmorpholin-4-yl]pyrido[5,6-e]pyrimidin-7-yl]-1,3-dihydroindol-2-one;
3-[2,4-bis[3-methylmorpholin-4-yl]pyrido[5,6-e]pyrimidin-7-yl]-N-methylbenzamide;
5-[2,4-bis[3-methylmorpholin-4-yl]pyrido[5,6-e]pyrimidin-7-yl]-2-(difluoromethoxy)benzamide;
6-[2,4-bis[3-methylmorpholin-4-yl]pyrido[5,6-e]pyrimidin-7-yl]-2,3-dihydroisoindol-1-one;
[5-[2-(2,6-dimethylmorpholin-4-yl)-4-morpholin-4-ylpyrido[6,5-d]pyrimidin-7-yl]-2-methoxyphenyl]methanol; and
[2-methoxy-5-[2-(3-methylmorpholin-4-yl)-4-morpholin-4-ylpyrido[6,5-d]pyrimidin-7-yl]phenyl]methanol.

4. A combination product according to claim 20 which comprises a kit of parts comprising the following components:
4-(4-fluoro-2-methyl-1H-indol-5-yloxy)-6-methoxy-7-(3-(pyrrolidin-1-yl)propoxy)quinazoline, or a pharmaceutically acceptable salt thereof, in association with a pharmaceutically acceptable adjuvant, diluent or carrier; and
a mTOR-selective kinase inhibitor, or a pharmaceutically acceptable salt thereof, in association with a pharmaceutically acceptable adjuvant, diluent or carrier,
wherein the components are provided in a form which is suitable for sequential, separate and/or simultaneous administration, and further comprising instructions to administer the components sequentially, separately and/or simultaneously,
and wherein the mTOR-selective kinase inhibitor is selected from any one of
5-[2,4-bis[3-methylmorpholin-4-yl]pyrido[5,6-e]pyrimidin-7-yl]-2-methoxy-N-methylbenzamide;
4-[2,4-bis[3-methylmorpholin-4-yl]pyrido[5,6-e]pyrimidin-7-yl]aniline;
6-[2,4-bis[3-methylmorpholin-4-yl]pyrido[5,6-e]pyrimidin-7-yl]-1H-indazol-3-amine;
8-[2,4-bis[3-methylmorpholin-4-yl]pyrido[5,6-e]pyrimidin-7-yl]-1,2,3,4-tetrahydro-1,4-benzodiazepin-5-one;
5-[2,4-bis[3-methylmorpholin-4-yl]pyrido[5,6-e]pyrimidin-7-yl]-2-methoxy-N-methylbenzamide;
5-[2,4-bis[3-methylmorpholin-4-yl]pyrido[5,6-e]pyrimidin-7-yl]pyridin-2-amine;
N-[3-[2,4-bis[3-methylmorpholin-4-yl]pyrido[5,6-e]pyrimidin-7-yl]phenyl]methanesulfonamide;
3-[2,4-bis[3-methylmorpholin-4-yl]pyrido[5,6-e]pyrimidin-7-yl]aniline;
5-[2,4-bis[3-methylmorpholin-4-yl]pyrido[6,5-d]pyrimidin-7-yl]-2-ethoxybenzamide;
5-[2,4-bis[3-methylmorpholin-4-yl]pyrido[5,6-e]pyrimidin-7-yl]-2-(difluoromethoxy)-N-methylbenzamide;
5-[2,4-bis[3-methylmorpholin-4-yl]pyrido[5,6-e]pyrimidin-7-yl]-1H-indazol-3-amine;
[5-[2,4-bis[3-methylmorpholin-4-yl]pyrido[5,6-e]pyrimidin-7-yl]-2-methoxyphenyl]methanol;
N-[[4-[2,4-bis[3-methylmorpholin-4-yl]pyrido[5,6-e]pyrimidin-7-yl]phenyl]methyl]methanesulfonamide;
5-[2,4-bis[3-methylmorpholin-4-yl]pyrido[5,6-e]pyrimidin-7-yl]-1,3-dihydroindol-2-one;
6-[2,4-bis[3-methylmorpholin-4-yl]pyrido[5,6-e]pyrimidin-7-yl]-1,3-dihydroindol-2-one;
3-[2,4-bis[3-methylmorpholin-4-yl]pyrido[5,6-e]pyrimidin-7-yl]-N-methylbenzamide;
5-[2,4-bis[3-methylmorpholin-4-yl]pyrido[5,6-e]pyrimidin-7-yl]-2-(difluoromethoxy)benzamide;
6-[2,4-bis[3-methylmorpholin-4-yl]pyrido[5,6-e]pyrimidin-7-yl]-2,3-dihydroisoindol-1-one;
[5-[2-(2,6-dimethylmorpholin-4-yl)-4-morpholin-4-ylpyrido[6,5-d]pyrimidin-7-yl]-2-methoxyphenyl]methanol; and
[2-methoxy-5-[2-(3-methylmorpholin-4-yl)-4-morpholin-4-ylpyrido[6,5-d]pyrimidin-7-yl]phenyl]methanol.

5. A combination product according to claim 20 which comprises a kit of parts comprising the following components:
4-(4-bromo-2-fluoroanilino)-6-methoxy-7-(1-methylpiperidin-4-ylmethoxy)quinazoline, or a pharmaceutically acceptable salt thereof, in association with a pharmaceutically acceptable adjuvant, diluent or carrier; and
a mTOR-selective kinase inhibitor, or a pharmaceutically acceptable salt thereof, in association with a pharmaceutically acceptable adjuvant, diluent or carrier,
wherein the components are provided in a form which is suitable for sequential, separate and/or simultaneous administration, and further comprising instructions to administer the components sequentially, separately and/or simultaneously,
and wherein the mTOR-selective kinase inhibitor is selected from any one of
5-[2,4-bis[3-methylmorpholin-4-yl]pyrido[5,6-e]pyrimidin-7-yl]-2-methoxy-N-methylbenzamide;
4-[2,4-bis[3-methylmorpholin-4-yl]pyrido[5,6-e]pyrimidin-7-yl]aniline;
6-[2,4-bis[3-methylmorpholin-4-yl]pyrido[5,6-e]pyrimidin-7-yl]-1H-indazol-3-amine;
8-[2,4-bis[3-methylmorpholin-4-yl]pyrido[5,6-e]pyrimidin-7-yl]-1,2,3,4-tetrahydro-1,4-benzodiazepin-5-one;
5-[2,4-bis[3-methylmorpholin-4-yl]pyrido[5,6-e]pyrimidin-7-yl]-2-methoxy-N-methylbenzamide;
5-[2,4-bis[3-methylmorpholin-4-yl]pyrido[5,6-e]pyrimidin-7-yl]pyridin-2-amine;
N-[3-[2,4-bis[3-methylmorpholin-4-yl]pyrido[5,6-e]pyrimidin-7-yl]phenyl]methanesulfonamide;
3-[2,4-bis[3-methylmorpholin-4-yl]pyrido[5,6-e]pyrimidin-7-yl]aniline;
5-[2,4-bis[3-methylmorpholin-4-yl]pyrido[6,5-d]pyrimidin-7-yl]-2-ethoxybenzamide;
5-[2,4-bis[3-methylmorpholin-4-yl]pyrido[5,6-e]pyrimidin-7-yl]-2-(difluoromethoxy)-N-methylbenzamide;
5-[2,4-bis[3-methylmorpholin-4-yl]pyrido[5,6-e]pyrimidin-7-yl]-1H-indazol-3-amine;
[5-[2,4-bis[3-methylmorpholin-4-yl]pyrido[5,6-e]pyrimidin-7-yl]-2-methoxyphenyl]methanol;
N-[[4-[2,4-bis[3-methylmorpholin-4-yl]pyrido[5,6-e]pyrimidin-7-yl]phenyl]methyl]methanesulfonamide;
5-[2,4-bis[3-methylmorpholin-4-yl]pyrido[5,6-e]pyrimidin-7-yl]-1,3-dihydroindol-2-one;
6-[2,4-bis[3-methylmorpholin-4-yl]pyrido[5,6-e]pyrimidin-7-yl]-1,3-dihydroindol-2-one;
3-[2,4-bis[3-methylmorpholin-4-yl]pyrido[5,6-e]pyrimidin-7-yl]-N-methylbenzamide;
5-[2,4-bis[3-methylmorpholin-4-yl]pyrido[5,6-e]pyrimidin-7-yl]-2-(difluoromethoxy)benzamide;
6-[2,4-bis[3-methylmorpholin-4-yl]pyrido[5,6-e]pyrimidin-7-yl]-2,3-dihydroisoindol-1-one;
[5-[2-(2,6-dimethylmorpholin-4-yl)-4-morpholin-4-ylpyrido[6,5-d]pyrimidin-7-yl]-2-methoxyphenyl]methanol; and
[2-methoxy-5-[2-(3-methylmorpholin-4-yl)-4-morpholin-4-ylpyrido[6,5-d]pyrimidin-7-yl]phenyl]methanol.

6. (canceled)

7. A combination product according to claim 5 wherein the mTOR-selective kinase inhibitor is [5-[2,4-bis[(3S)-3-methylmorpholin-4-yl]pyrido[5,6-e]pyrimidin-7-yl]-2-methoxyphenyl]methanol.

8. (canceled)

9. (canceled)

10. (canceled)

11. A method of treating cancer according to claim 22, which comprises administration of 4-(4-fluoro-2-methyl-1H-indol-5-yloxy)-6-methoxy-7-(3-(pyrrolidin-1-yl)propoxy)quinazoline, or a pharmaceutically acceptable salt thereof, and a mTOR-selective kinase inhibitor, or a pharmaceutically acceptable salt thereof, to a patient, having or suspected of having cancer.

12. A method of treating cancer according to claim 22, which comprises administration of 4-(4-bromo-2-fluoroanilino)-6-methoxy-7-(1-methylpiperidin-4-ylmethoxy)quinazoline, or a pharmaceutically acceptable salt thereof, and a mTOR-selective kinase inhibitor, or a pharmaceutically acceptable salt thereof, to a patient, having or suspected of having cancer.

13. A method of treating cancer according to claim 12 wherein the cancer is selected from lung cancer, prostate cancer, melanoma, ovarian cancer, breast cancer, endometrial cancer, kidney cancer, sarcomas and head and neck cancers, gastric cancer, tumours of the central nervous system.

14. (canceled)

15. (canceled)

16. A method of treating cancer according to claim 11 wherein the mTOR-selective kinase inhibitor, or a pharmaceutically-acceptable salt thereof,
wherein the mTOR-selective kinase inhibitor is selected from any one of
5-[2,4-bis[3-methylmorpholin-4-yl]pyrido[5,6-e]pyrimidin-7-yl]-2-methoxy-N-methylbenzamide;
4-[2,4-bis[3-methylmorpholin-4-yl]pyrido[5,6-e]pyrimidin-7-yl]aniline;
6-[2,4-bis[3-methylmorpholin-4-yl]pyrido[5,6-e]pyrimidin-7-yl]-1H-indazol-3-amine;
8-[2,4-bis[3-methylmorpholin-4-yl]pyrido[5,6-e]pyrimidin-7-yl]-1,2,3,4-tetrahydro-1,4-benzodiazepin-5-one;
5-[2,4-bis[3-methylmorpholin-4-yl]pyrido[5,6-e]pyrimidin-7-yl]-2-methoxy-N-methylbenzamide;
5-[2,4-bis[3-methylmorpholin-4-yl]pyrido[5,6-e]pyrimidin-7-yl]pyridin-2-amine;
N-[3-[2,4-bis[3-methylmorpholin-4-yl]pyrido[5,6-e]pyrimidin-7-yl]phenyl]methanesulfonamide;
3-[2,4-bis[3-methylmorpholin-4-yl]pyrido[5,6-e]pyrimidin-7-yl]aniline;
5-[2,4-bis[3-methylmorpholin-4-yl]pyrido[6,5-d]pyrimidin-7-yl]-2-ethoxybenzamide;
5-[2,4-bis[3-methylmorpholin-4-yl]pyrido[5,6-e]pyrimidin-7-yl]-2-(difluoromethoxy)-N-methylbenzamide;
5-[2,4-bis[3-methylmorpholin-4-yl]pyrido[5,6-e]pyrimidin-7-yl]-1H-indazol-3-amine;
[5-[2,4-bis[3-methylmorpholin-4-yl]pyrido[5,6-e]pyrimidin-7-yl]-2-methoxyphenyl]methanol;
N-[[4-[2,4-bis[3-methylmorpholin-4-yl]pyrido[5,6-e]pyrimidin-7-yl]phenyl]methyl]methanesulfonamide;
5-[2,4-bis[3-methylmorpholin-4-yl]pyrido[5,6-e]pyrimidin-7-yl]-1,3-dihydroindol-2-one;
6-[2,4-bis[3-methylmorpholin-4-yl]pyrido[5,6-e]pyrimidin-7-yl]-1,3-dihydroindol-2-one;
3-[2,4-bis[3-methylmorpholin-4-yl]pyrido[5,6-e]pyrimidin-7-yl]-N-methylbenzamide;
5-[2,4-bis[3-methylmorpholin-4-yl]pyrido[5,6-e]pyrimidin-7-yl]-2-(difluoromethoxy)benzamide;
6-[2,4-bis[3-methylmorpholin-4-yl]pyrido[5,6-e]pyrimidin-7-yl]-2,3-dihydroisoindol-1-one;
[5-[2-(2,6-dimethylmorpholin-4-yl)-4-morpholin-4-ylpyrido[6,5-d]pyrimidin-7-yl]-2-methoxyphenyl]methanol; and
[2-methoxy-5-[2-(3-methylmorpholin-4-yl)-4-morpholin-4-ylpyrido[6,5-d]pyrimidin-7-yl]phenyl]methanol.

17. (canceled)

18. A combination product according to claim 2 wherein the mTOR-selective kinase inhibitor is [5-[2,4-bis[(3S)-3-methylmorpholin-4-yl]pyrido[5,6-e]pyrimidin-7-yl]-2-methoxyphenyl]methanol.

19. A combination product according to claim 3 wherein the mTOR-selective kinase inhibitor is [5-[2,4-bis[(3S)-3-methylmorpholin-4-yl]pyrido[5,6-e]pyrimidin-7-yl]-2-methoxyphenyl]methanol.

20. A combination product which comprises a kit of parts comprising the following components:
a VEGFR tyrosine kinase inhibitor, or a pharmaceutically acceptable salt thereof, in association with a pharmaceutically acceptable adjuvant, diluent or carrier, and
a mTOR-selective kinase inhibitor, or a pharmaceutically acceptable salt thereof, in association with a pharmaceutically acceptable adjuvant, diluent or carrier,
wherein the components are provided in a form which is suitable for sequential, separate and/or simultaneous administration, and further comprising instructions to administer the components sequentially, separately and/or simultaneously.

21. A combination product according to claim 4 wherein the mTOR-selective kinase inhibitor is [5-[2,4-bis[(3S)-3-methylmorpholin-4-yl]pyrido[5,6-e]pyrimidin-7-yl]-2-methoxyphenyl]methanol.

22. A method of treating cancer, which comprises administration of a VEGFR tyrosine kinase inhibitor, or a pharmaceutically acceptable salt thereof, and a mTOR-selective kinase inhibitor, or a pharmaceutically acceptable salt thereof, to a patient, having or suspected of having cancer.

23. A method of treating cancer according to claim 11 wherein the mTOR-selective kinase inhibitor is [5-[2,4-bis[(3S)-3-methylmorpholin-4-yl]pyrido[5,6-e]pyrimidin-7-yl]-2-methoxyphenyl]methanol.

24. A method of treating cancer according to claim 12 wherein the cancer is selected from lung cancer, prostate cancer, melanoma, ovarian cancer, breast cancer, endometrial cancer, kidney cancer, sarcomas and head and neck cancers, gastric cancer, tumours of the central nervous system.

25. A method of treating cancer according to claim 11 wherein the mTOR-selective kinase inhibitor, or a pharmaceutically-acceptable salt thereof,
wherein the mTOR-selective kinase inhibitor is selected from any one of
5-[2,4-bis[3-methylmorpholin-4-yl]pyrido[5,6-e]pyrimidin-7-yl]-2-methoxy-N-methylbenzamide;
4-[2,4-bis[3-methylmorpholin-4-yl]pyrido[5,6-e]pyrimidin-7-yl]aniline;
6-[2,4-bis[3-methylmorpholin-4-yl]pyrido[5,6-e]pyrimidin-7-yl]-1H-indazol-3-amine;
8-[2,4-bis[3-methylmorpholin-4-yl]pyrido[5,6-e]pyrimidin-7-yl]-1,2,3,4-tetrahydro-1,4-benzodiazepin-5-one;
5-[2,4-bis[3-methylmorpholin-4-yl]pyrido[5,6-e]pyrimidin-7-yl]-2-methoxy-N-methylbenzamide;
5-[2,4-bis[3-methylmorpholin-4-yl]pyrido[5,6-e]pyrimidin-7-yl]pyridin-2-amine;
N-[3-[2,4-bis[3-methylmorpholin-4-yl]pyrido[5,6-e]pyrimidin-7-yl]phenyl]methanesulfonamide;
3-[2,4-bis[3-methylmorpholin-4-yl]pyrido[5,6-e]pyrimidin-7-yl]aniline;
5-[2,4-bis[3-methylmorpholin-4-yl]pyrido[6,5-d]pyrimidin-7-yl]-2-ethoxybenzamide;
5-[2,4-bis[3-methylmorpholin-4-yl]pyrido[5,6-e]pyrimidin-7-yl]-2-(difluoromethoxy)-N-methylbenzamide;
5-[2,4-bis[3-methylmorpholin-4-yl]pyrido[5,6-e]pyrimidin-7-yl]-1H-indazol-3-amine;
[5-[2,4-bis[3-methylmorpholin-4-yl]pyrido[5,6-e]pyrimidin-7-yl]-2-methoxyphenyl]methanol;
N-[[4-[2,4-bis[3-methylmorpholin-4-yl]pyrido[5,6-e]pyrimidin-7-yl]phenyl]methyl]methanesulfonamide;
5-[2,4-bis[3-methylmorpholin-4-yl]pyrido[5,6-e]pyrimidin-7-yl]-1,3-dihydroindol-2-one;
6-[2,4-bis[3-methylmorpholin-4-yl]pyrido[5,6-e]pyrimidin-7-yl]-1,3-dihydroindol-2-one;
3-[2,4-bis[3-methylmorpholin-4-yl]pyrido[5,6-e]pyrimidin-7-yl]-N-methylbenzamide;
5-[2,4-bis[3-methylmorpholin-4-yl]pyrido[5,6-e]pyrimidin-7-yl]-2-(difluoromethoxy)benzamide;
6-[2,4-bis[3-methylmorpholin-4-yl]pyrido[5,6-e]pyrimidin-7-yl]-2,3-dihydroisoindol-1-one;
[5-[2-(2,6-dimethylmorpholin-4-yl)-4-morpholin-4-ylpyrido[6,5-d]pyrimidin-7-yl]-2-methoxyphenyl]methanol; and
[2-methoxy-5-[2-(3-methylmorpholin-4-yl)-4-morpholin-4-ylpyrido[6,5-d]pyrimidin-7-yl]phenyl]methanol.

26. A method of treating cancer according to claim 12 wherein the mTOR-selective kinase inhibitor is [5-[2,4-bis[(3S)-3-methylmorpholin-4-yl]pyrido[5,6-e]pyrimidin-7-yl]-2-methoxyphenyl]methanol.
